SEQUENCE LISTING (132 sequences)

SEQ ID NO: 1 - protein, 56 aa, Artificial Sequence
  Consensus sequence Genotype G and vaccine strains 2-57
  MISC_FEATURE (4)..(4): X is Q or R
  MISC_FEATURE (7)..(7): X is L or S
  MISC_FEATURE (8)..(8): X is Y or H
  MISC_FEATURE (9)..(9): X is L or P
  MISC_FEATURE (15)..(15): X is I or T
  MISC_FEATURE (18)..(18): X is Y or S
  MISC_FEATURE (21)..(21): X is I, T or V
  MISC_FEATURE (27)..(27): X is I or T
  MISC_FEATURE (28)..(28): X is I or T
  MISC_FEATURE (29)..(29): X is L or S
  MISC_FEATURE (30)..(30): X is T or A
  MISC_FEATURE (31)..(31): X is V or I
  MISC_FEATURE (32)..(32): X is T or N
  MISC_FEATURE (33)..(33): X is Y or H
  MISC_FEATURE (34)..(34): X is K or N
  MISC_FEATURE (36)..(36): X is A or V
  MISC_FEATURE (39)..(39): X is H, P, Y or R
  MISC_FEATURE (41)..(41): X is A, T, or S
  MISC_FEATURE (43)..(43): X is Y or H
  MISC_FEATURE (47)..(47): X is F, C or S
  MISC_FEATURE (48)..(48): X is F, V or S
  MISC_FEATURE (49)..(49): X is H or R
  MISC_FEATURE (51)..(51): X is S, R or G
  MISC_FEATURE (52)..(52): X is F or L
  MISC_FEATURE (54)..(54): X is H or Q
  MISC_FEATURE (55)..(55): X is S, P, or T
PAIXPPXXXTFLLLXLLXLIXTLYVWXXXXXXXXTXVRXAXLXQRSXXXWXXDXXL

SEQ ID NO: 2 - protein, 56 aa, Artificial Sequence
  Consensus sequence Genotype G 2-57
  MISC_FEATURE (4)..(4): X is Q or R
  MISC_FEATURE (7)..(7): X is L or S
  MISC_FEATURE (21)..(21): X is I or T
  MISC_FEATURE (30)..(30): X is T or A
  MISC_FEATURE (39)..(39): X is H, P or R
  MISC_FEATURE (47)..(47): X is F or S
  MISC_FEATURE (48)..(48): X is F or V
  MISC_FEATURE (51)..(51): X is S or R
  MISC_FEATURE (55)..(55): X is S or T
PAIXPPXYLTFLLLILLYLIXTLYVWIILXVTYKTAVRXAALYQRSXXHWXFDHXL

SEQ ID NO: 3 - protein, 56 aa, Artificial Sequence
  Consensus sequence Vaccine strains 2-57
  MISC_FEATURE (8)..(8): X is Y or H
  MISC_FEATURE (9)..(9): X is L or P
  MISC_FEATURE (15)..(15): X is I or T
  MISC_FEATURE (18)..(18): X is Y or S
  MISC_FEATURE (21)..(21): X is I or V
  MISC_FEATURE (27)..(27): X is I or T
  MISC_FEATURE (28)..(28): X is I or T
  MISC_FEATURE (29)..(29): X is L or S
  MISC_FEATURE (32)..(32): X is T or N
  MISC_FEATURE (33)..(33): X is Y or H
  MISC_FEATURE (34)..(34): X is K or N
  MISC_FEATURE (36)..(36): X is A or V
  MISC_FEATURE (39)..(39): X is Y or H
  MISC_FEATURE (41)..(41): X is A, T, or S
  MISC_FEATURE (43)..(43): X is H or Y
  MISC_FEATURE (47)..(47): X is F or C
  MISC_FEATURE (48)..(48): X is F or S
  MISC_FEATURE (51)..(51): X is S or G
  MISC_FEATURE (52)..(52): X is F or L
  MISC_FEATURE (54)..(54): X is H or Q
  MISC_FEATURE (55)..(55): X is S or P
PAIQPPLXXTFLLLXLLXLIXTLYVWXXXTIXXXTXVRXAXLXQRSXXRWXXDXXL

SEQ ID NO: 4 - protein, 57 aa, Artificial Sequence
  Consensus sequence Genotype G and vaccine strains Full length
  MISC_FEATURE (5)..(5): X is Q or R
  MISC_FEATURE (8)..(8): X is L or S
  MISC_FEATURE (9)..(9): X is Y or H
  MISC_FEATURE (10)..(10): X is L or P
  MISC_FEATURE (16)..(16): X is I or T
  MISC_FEATURE (19)..(19): X is Y or S
  MISC_FEATURE (22)..(22): X is I, T or V
  MISC_FEATURE (28)..(28): X is I or T
  MISC_FEATURE (29)..(29): X is I or T
  MISC_FEATURE (30)..(30): X is L or S
  MISC_FEATURE (31)..(31): X is T or A
  MISC_FEATURE (32)..(32): X is V or I
  MISC_FEATURE (33)..(33): X is T or N
  MISC_FEATURE (34)..(34): X is Y or H
  MISC_FEATURE (35)..(35): X is K or N
  MISC_FEATURE (37)..(37): X is A or V
  MISC_FEATURE (40)..(40): X is H, P, Y or R
  MISC_FEATURE (42)..(42): X is A, T, or S
  MISC_FEATURE (44)..(44): X is Y or H
  MISC_FEATURE (48)..(48): X is F, C or S
  MISC_FEATURE (49)..(49): X is F, V or S
  MISC_FEATURE (50)..(50): X is H or R
  MISC_FEATURE (52)..(52): X is S, R or G
  MISC_FEATURE (53)..(53): X is F or L
  MISC_FEATURE (55)..(55): X is H or Q
  MISC_FEATURE (56)..(56): X is S, P, or T
MPAIXPPXXXTFLLLXLLXLIXTLYVWXXXXXXXXTXVRXAXLXQRSXXXWXXDXXL

SEQ ID NO: 5 - protein, 57 aa, Artificial Sequence
  Consensus sequence Genotype G Full length
  MISC_FEATURE (5)..(5): X is Q or R
  MISC_FEATURE (8)..(8): X is L or S
  MISC_FEATURE (22)..(22): X is I or T
  MISC_FEATURE (31)..(31): X is T or A
  MISC_FEATURE (40)..(40): X is H, P or R
  MISC_FEATURE (48)..(48): X is F or S
  MISC_FEATURE (49)..(49): X is F or V
  MISC_FEATURE (52)..(52): X is S or R
  MISC_FEATURE (56)..(56): X is S or T
MPAIXPPXYLTFLLLILLYLIXTLYVWIILXVTYKTAVRXAALYQRSXXHWXFDHXL

SEQ ID NO: 6 - protein, 57 aa, Artificial Sequence
  Consensus sequence Vaccine strains Full length
  MISC_FEATURE (9)..(9): X is Y or H
  MISC_FEATURE (10)..(10): X is L or P
  MISC_FEATURE (16)..(16): X is I or T
  MISC_FEATURE (19)..(19): X is Y or S
  MISC_FEATURE (22)..(22): X is I or V
  MISC_FEATURE (28)..(28): X is I or T
  MISC_FEATURE (29)..(29): X is I or T
  MISC_FEATURE (30)..(30): X is L or S
  MISC_FEATURE (33)..(33): X is T or N
  MISC_FEATURE (34)..(34): X is Y or H
  MISC_FEATURE (35)..(35): X is K or N
  MISC_FEATURE (37)..(37): X is A or V
  MISC_FEATURE (40)..(40): X is Y or H
  MISC_FEATURE (42)..(42): X is A, T, or S
  MISC_FEATURE (44)..(44): X is H or Y
  MISC_FEATURE (48)..(48): X is F or C
  MISC_FEATURE (49)..(49): X is F or S
  MISC_FEATURE (52)..(52): X is S or G
  MISC_FEATURE (53)..(53): X is F or L
  MISC_FEATURE (55)..(55): X is H or Q
  MISC_FEATURE (56)..(56): X is S or P
MPAIQPPLXXTFLLLXLLXLIXTLYVWXXXTIXXXTXVRXAXLXQRSXXRWXXDXXL

SEQ ID NO: 7 - protein, 15 aa, Artificial Sequence
  Consensus sequence Genotype G and vaccine strains 1-15
  MISC_FEATURE (5)..(5): X is Q or R
  MISC_FEATURE (8)..(8): X is L or S
  MISC_FEATURE (9)..(9): X is Y or H
  MISC_FEATURE (10)..(10): X is L or P
MPAIXPPXXXTFLLL

SEQ ID NO: 8 - protein, 15 aa, Artificial Sequence
  Concensus sequence Genotype G 1-15
  MISC_FEATURE (5)..(5): X is Q or R
  MISC_FEATURE (8)..(8): X is L or S
MPAIXPPXYLTFLLL

SEQ ID NO: 9 - protein, 15 aa, Artificial Sequence
  Consensus sequence vaccine strains 1-15
  MISC_FEATURE (9)..(9): X is Y or H
  MISC_FEATURE (10)..(10): X is L or P
MPAIQPPLXXTFLLL

SEQ ID NO: 10 - protein, 14 aa, Artificial Sequence
  Consensus sequence Genotype G and vaccine strains 1-14
  MISC_FEATURE (5)..(5): X is Q or R
  MISC_FEATURE (8)..(8): X is L or S
  MISC_FEATURE (9)..(9): X is Y or H
  MISC_FEATURE (10)..(10): X is L or P
MPAIXPPXXXTFLL

SEQ ID NO: 11 - protein, 14 aa, Artificial Sequence
  Consensus sequence Genotype G 1-14
  MISC_FEATURE (5)..(5): X is Q or R
  MISC_FEATURE (8)..(8): X is L or S
MPAIXPPXYLTFLL

SEQ ID NO: 12 - protein, 14 aa, Artificial Sequence
  Consensus sequence Vaccine strains 1-14
  MISC_FEATURE (9)..(9): X is Y or H
  MISC_FEATURE (10)..(10): X is L or P
MPAIQPPLXXTFLL

SEQ ID NO: 13 - protein, 13 aa, Artificial Sequence
  Consensus sequence Genotype G and vaccine strains 1-13
  MISC_FEATURE (5)..(5): X is Q or R
  MISC_FEATURE (8)..(8): X is L or S
  MISC_FEATURE (9)..(9): X is Y or H
  MISC_FEATURE (10)..(10): X is L or P
MPAIXPPXXXTFL

SEQ ID NO: 14 - protein, 13 aa, Artificial Sequence
  Consensus sequence Genotype G 1-13
  MISC_FEATURE (5)..(5): X is Q or R
  MISC_FEATURE (8)..(8): X is L or S
MPAIXPPXYLTFL

SEQ ID NO: 15 - protein, 13 aa, Artificial Sequence
  Consensus sequence Vaccine strain 1-13
  MISC_FEATURE (9)..(9): X is Y or H
  MISC_FEATURE (10)..(10): X is L or P
MPAIQPPLXXTFL

SEQ ID NO: 16 - protein, 12 aa, Artificial Sequence
  Consensus sequence Genotype G and vaccine strains 1-12
  MISC_FEATURE (5)..(5): X is Q or R
  MISC_FEATURE (8)..(8): X is L or S
  MISC_FEATURE (9)..(9): X is Y or H
  MISC_FEATURE (10)..(10): X is L or P
MPAIXPPXXXTF

SEQ ID NO: 17 - protein, 12 aa, Artificial Sequence
  Consensus sequence Genotype G 1-12
  MISC_FEATURE (5)..(5): X is Q or R
  MISC_FEATURE (8)..(8): X is L or S
MPAIXPPXYLTF

SEQ ID NO: 18 - protein, 12 aa, Artificial Sequence
  Consensus sequence Vaccine strain 1-12
  MISC_FEATURE (9)..(9): X is Y or H
  MISC_FEATURE (10)..(10): X is L or P
MPAIQPPLXXTF

SEQ ID NO: 19 - protein, 11 aa, Artificial Sequence
  Consensus sequence Genotype G and vaccine strain 1-11
  MISC_FEATURE (5)..(5): X is Q or R
  MISC_FEATURE (8)..(8): X is L or S
  MISC_FEATURE (9)..(9): X is Y or H
  MISC_FEATURE (10)..(10): X is L or P
MPAIXPPXXXT

SEQ ID NO: 20 - protein, 11 aa, Artificial Sequence
  Consensus sequence Genotype G 1-11
  MISC_FEATURE (5)..(5): X is Q or R
  MISC_FEATURE (8)..(8): X is L or S
MPAIXPPXYLT

SEQ ID NO: 21 - protein, 11 aa, Artificial Sequence
  Consensus sequence Vaccine strains 1-11
  MISC_FEATURE (9)..(9): X is Y or H
  MISC_FEATURE (10)..(10): X is L or P
MPAIQPPLXXT

SEQ ID NO: 22 - protein, 10 aa, Artificial Sequence
  Consensus sequence Genotypr G and vaccine strains 1-10
  MISC_FEATURE (5)..(5): X is Q or R
  MISC_FEATURE (8)..(8): X is L or S
  MISC_FEATURE (9)..(9): X is Y or H
  MISC_FEATURE (10)..(10): X is L or P
MPAIXPPXXX

SEQ ID NO: 23 - protein, 10 aa, Artificial Sequence
  Consensus sequence Genotype G 1-10
  MISC_FEATURE (5)..(5): X is Q or R
  MISC_FEATURE (8)..(8): X is L or S
MPAIXPPXYL

SEQ ID NO: 24 - protein, 10 aa, Artificial Sequence
  Consensus sequence VAccine strains 1-10
  MISC_FEATURE (9)..(9): X is Y or H
  MISC_FEATURE (10)..(10): X is L or P
MPAIQPPLXX

SEQ ID NO: 25 - protein, 9 aa, Artificial Sequence
  Consensus sequence Genotype G and vaccine strains 1-9
  MISC_FEATURE (5)..(5): X is Q or R
  MISC_FEATURE (8)..(8): X is L or S
  MISC_FEATURE (9)..(9): X is Y or H
MPAIXPPXX

SEQ ID NO: 26 - protein, 9 aa, Artificial Sequence
  Consensus sequence vaccine strains 1-9
  MISC_FEATURE (9)..(9): X is Y or H
MPAIQPPLX

SEQ ID NO: 27 - protein, 8 aa, Artificial Sequence
  Consensus sequence Genotype G and vaccine strains 1-8
  MISC_FEATURE (5)..(5): X is Q or R
  MISC_FEATURE (8)..(8): X is L or S
MPAIXPPX

SEQ ID NO: 28 - protein, 8 aa, Artificial Sequence
  Vaccine strains and Genotype G Genbank ID QFO38283.1 1-8
MPAIQPPL

SEQ ID NO: 29 - protein, 7 aa, Artificial Sequence
  Consensus sequence Genotype G and vaccine strains 1-7
  MISC_FEATURE (5)..(5): X is Q or R
MPAIXPP

SEQ ID NO: 30 - protein, 7 aa, Artificial Sequence
  Vaccine strains and Genotype G Genbank ID QFO38283.1 1-7
MPAIQPP

SEQ ID NO: 31 - protein, 6 aa, Artificial Sequence
  Consensus sequence Genotype G and vaccine strains 1-6
  MISC_FEATURE (5)..(5): X is Q or R
MPAIXP

SEQ ID NO: 32 - protein, 6 aa, Artificial Sequence
  Vaccine strains and Genotype G Genbank ID QFO38283.1 1-6
MPAIQP

SEQ ID NO: 33 - protein, 5 aa, Artificial Sequence
  Consensus sequence Genotype G and vaccine strains 1-5
  MISC_FEATURE (5)..(5): X is Q or R
MPAIX

SEQ ID NO: 34 - protein, 5 aa, Artificial Sequence
  Vaccine strains and Genotype G Genbank ID QFO38283.1 1-5
MPAIQ

SEQ ID NO: 35 - protein, 14 aa, Artificial Sequence
  Consensus sequence Genotype G and vaccine strains 2-15
  MISC_FEATURE (4)..(4): X is Q or R
  MISC_FEATURE (7)..(7): X is L or S
  MISC_FEATURE (8)..(8): X is Y or H
  MISC_FEATURE (9)..(9): X is L or P
PAIXPPXXXTFLLL

SEQ ID NO: 36 - protein, 14 aa, Artificial Sequence
  Consensus sequence Genotype G 2-15
  MISC_FEATURE (4)..(4): X is Q or R
  MISC_FEATURE (7)..(7): X is L or S
PAIXPPXYLTFLLL

SEQ ID NO: 37 - protein, 14 aa, Artificial Sequence
  Consensus sequence Vaccine strains 2-15
  MISC_FEATURE (8)..(8): X is Y or H
  MISC_FEATURE (9)..(9): X is L or P
PAIQPPLXXTFLLL

SEQ ID NO: 38 - protein, 13 aa, Artificial Sequence
  Consensus sequence Genotype G and vaccine strains 2-14
  MISC_FEATURE (4)..(4): X is Q or R
  MISC_FEATURE (7)..(7): X is L or S
  MISC_FEATURE (8)..(8): X is Y or H
  MISC_FEATURE (9)..(9): X is L or P
PAIXPPXXXTFLL

SEQ ID NO: 39 - protein, 13 aa, Artificial Sequence
  Consensus sequence Genotype G 2-14
  MISC_FEATURE (4)..(4): X is Q or R
  MISC_FEATURE (7)..(7): X is L or S
PAIXPPXYLTFLL

SEQ ID NO: 40 - protein, 13 aa, Artificial Sequence
  Consensus sequence Vaccine strains 2-14
  MISC_FEATURE (8)..(8): X is Y or H
  MISC_FEATURE (9)..(9): X is L or P
PAIQPPLXXTFLL

SEQ ID NO: 41 - protein, 12 aa, Artificial Sequence
  Consensus sequence Genotype G and vaccine strains 2-13
  MISC_FEATURE (4)..(4): X is Q or R
  MISC_FEATURE (7)..(7): X is L or S
  MISC_FEATURE (8)..(8): X is Y or H
  MISC_FEATURE (9)..(9): X is L or P
PAIXPPXXXTFL

SEQ ID NO: 42 - protein, 12 aa, Artificial Sequence
  Consensus sequence Genotype G 2-13
  MISC_FEATURE (4)..(4): X is Q or R
  MISC_FEATURE (7)..(7): X is L or S
PAIXPPXYLTFL

SEQ ID NO: 43 - protein, 12 aa, Artificial Sequence
  Consensus sequence Vaccine strains 2-13
  MISC_FEATURE (8)..(8): X is Y or H
  MISC_FEATURE (8)..(8): X is L or P
  misc_feature (9)..(9): Xaa can be any naturally occurring amino acid
PAIQPPLXXTFL

SEQ ID NO: 44 - protein, 11 aa, Artificial Sequence
  Consensus sequence Genotype G and vaccine strains 2-12
  MISC_FEATURE (4)..(4): X is Q or R
  MISC_FEATURE (7)..(7): X is L or S
  MISC_FEATURE (8)..(8): X is Y or H
  MISC_FEATURE (9)..(9): X is L or P
PAIXPPXXXTF

SEQ ID NO: 45 - protein, 11 aa, Artificial Sequence
  Consensus sequence Genotype G 2-12
  MISC_FEATURE (4)..(4): X is Q or R
  MISC_FEATURE (7)..(7): X is L or S
PAIXPPXYLTF

SEQ ID NO: 46 - protein, 11 aa, Artificial Sequence
  Consensus sequence Vaccine strains 2-12
  MISC_FEATURE (8)..(8): X is Y or H
  MISC_FEATURE (9)..(9): X is L or P
PAIQPPLXXTF

SEQ ID NO: 47 - protein, 10 aa, Artificial Sequence
  Consensus sequnce Genotype G and vaccine strains 2-11
  MISC_FEATURE (4)..(4): X is Q or R
  MISC_FEATURE (7)..(7): X is L or S
  MISC_FEATURE (8)..(8): X is Y or H
  MISC_FEATURE (9)..(9): X is L or P
PAIXPPXXXT

SEQ ID NO: 48 - protein, 10 aa, Artificial Sequence
  Consensus sequence Genotype G 2-11
  MISC_FEATURE (4)..(4): X is Q or R
  MISC_FEATURE (7)..(7): X is L or S
PAIXPPXYLT

SEQ ID NO: 49 - protein, 10 aa, Artificial Sequence
  Consensus sequence Vaccine strains 2-11
  MISC_FEATURE (8)..(8): X is Y or H
  MISC_FEATURE (9)..(9): X is L or P
PAIQPPLXXT

SEQ ID NO: 50 - protein, 9 aa, Artificial Sequence
  Consensus sequence Genotype G and vaccine strains 2-10
  MISC_FEATURE (4)..(4): X is Q or R
  MISC_FEATURE (7)..(7): X is L or S
  MISC_FEATURE (8)..(8): X is Y or H
  MISC_FEATURE (9)..(9): X is L or P
PAIXPPXXX

SEQ ID NO: 51 - protein, 9 aa, Artificial Sequence
  Consensus sequence Genotype G 2-10
  MISC_FEATURE (4)..(4): X is Q or R
  MISC_FEATURE (7)..(7): X is L or S
PAIXPPXYL

SEQ ID NO: 52 - protein, 9 aa, Artificial Sequence
  Consensus sequence Vaccine strains 2-10
  MISC_FEATURE (8)..(8): X is Y or H
  MISC_FEATURE (9)..(9): X is L or P
PAIQPPLXX

SEQ ID NO: 53 - protein, 8 aa, Artificial Sequence
  Consensus sequence Genotype G and vaccine strains 2-9
  MISC_FEATURE (4)..(4): X is Q or R
  MISC_FEATURE (7)..(7): X is L or S
  MISC_FEATURE (8)..(8): X is Y or H
PAIXPPXX

SEQ ID NO: 54 - protein, 8 aa, Artificial Sequence
  Consensus sequence Vaccine strains 2-9
  MISC_FEATURE (8)..(8): X is Y or H
PAIQPPLX

SEQ ID NO: 55 - protein, 7 aa, Artificial Sequence
  Consensus sequence Genotype G and vaccine strains 2-8
  MISC_FEATURE (4)..(4): X is Q or R
  MISC_FEATURE (7)..(7): X is L or S
PAIXPPX

SEQ ID NO: 56 - protein, 7 aa, Artificial Sequence
  Vaccine strains and Genotype G Genbank ID QFO38283.1 2-8
PAIQPPL

SEQ ID NO: 57 - protein, 6 aa, Artificial Sequence
  Consensus sequence Genotype G and vaccine strains 2-7
  MISC_FEATURE (4)..(4): X is Q or R
PAIXPP

SEQ ID NO: 58 - protein, 6 aa, Artificial Sequence
  Vaccine strains and Genotype G Genbank ID QFO38283.1 2-7
PAIQPP

SEQ ID NO: 59 - protein, 5 aa, Artificial Sequence
  Consensus sequence Genotype G and vaccine strains 2-6
  MISC_FEATURE (4)..(4): X is Q or R
PAIXP

SEQ ID NO: 60 - protein, 5 aa, Artificial Sequence
  Vaccine strains and Genotype G Genbank ID QFO38283.1 2-6
PAIQP

SEQ ID NO: 61 - protein, 4 aa, Artificial Sequence
  Consensus sequence Genotype G and vaccine strains 2-5
  MISC_FEATURE (4)..(4): X is Q or R
PAIX

SEQ ID NO: 62 - protein, 4 aa, Artificial Sequence
  Vaccine strains  and Genotype G Genbank ID QFO38283.1 2-5
PAIQ

SEQ ID NO: 63 - protein, 56 aa, Artificial Sequence
  Genotype G Genbank ID QFO38283.1 2-57
PAIQPPLYLTFLLLILLYLIITLYVWIILTVTYKTAVRHAALYQRSFFHWSFDHSL

SEQ ID NO: 64 - protein, 56 aa, Artificial Sequence
  Jeryl Lynn strain or RIT 4385 strain 2-57
PAIQPPLYLTFLLLTLLYLIITLYVWTILTINHNTAVRHAALYQRSFSRWGFDQSL

SEQ ID NO: 65 - protein, 56 aa, Artificial Sequence
  Urabe AM9 strain 2-57
PAIQPPLYPTFLLLILLSLIVTLYVWIISTITYKTVVRHAALYQRSFFRWSFDHSL

SEQ ID NO: 66 - protein, 56 aa, Artificial Sequence
  Leningrad 3 strain 2-57
PAIQPPLYLTFLLLILLYLIITLYVWIILTITYKTAVRHAALHQRSFFRWSFDHSL

SEQ ID NO: 67 - protein, 56 aa, Artificial Sequence; identical to SEQ ID NO: 66
  Leningrad-Zagreb strain 2-57

SEQ ID NO: 68 - protein, 56 aa, Artificial Sequence
  S79 strain 2-57
PAIQPPLYLTFLLLTLLYLIITLYVWTILTINHNTAVRYAALYQRSFSRWGFDQSL

SEQ ID NO: 69 - protein, 56 aa, Artificial Sequence
  Rubini strain 2-57
PAIQPPLYLTFLLLILLYLIITLYVWTILTINHKTAVRYAALYQRSCSRWGFDQSL

SEQ ID NO: 70 - protein, 56 aa, Artificial Sequence
  Hishino strain 2-57
PAIQPPLYPTFLLLILLSLIITLYVWIISTITYKTAVRHAALYQRSFFRWSFDHSL

SEQ ID NO: 71 - protein, 56 aa, Artificial Sequence
  RS(S-12) strain 2-57
PAIQPPLHLTFLLLILLYLIITLYVWITLTITYKTAVRHATLYQRSFFRWSFDHPL

SEQ ID NO: 72 - protein, 56 aa, Artificial Sequence
  Torii strain 2-57
PAIQPPLYPTFLLLILLSLIITLYVWIISTITYKTAVRHASLYQRSFSRWSFDHSL

SEQ ID NO: 73 - protein, 56 aa, Artificial Sequence
  Mijahada strain 2-57
PAIQPPLYPTFLLLILLSLIITLYVWIISTITYKTAVRHAALHQRSFSRWSLDHSL

SEQ ID NO: 74 - protein, 57 aa, Mumps virus
MPAIQPPLYLTFLLLILLYLIITLYVWIILTVTYKTAVRHAALYQRSFFHWSFDHSL

SEQ ID NO: 75 - protein, 57 aa, Mumps virus
MPAIQPPLYLTFLLLTLLYLIITLYVWTILTINHNTAVRHAALYQRSFSRWGFDQSL

SEQ ID NO: 76 - protein, 57 aa, Mumps virus
MPAIQPPLYPTFLLLILLSLIVTLYVWIISTITYKTVVRHAALYQRSFFRWSFDHSL

SEQ ID NO: 77 - protein, 57 aa, Mumps virus
MPAIQPPLYLTFLLLILLYLIITLYVWIILTITYKTAVRHAALHQRSFFRWSFDHSL

SEQ ID NO: 78 - protein, 57 aa, Mumps virus; identical to SEQ ID NO: 77

SEQ ID NO: 79 - protein, 57 aa, Mumps virus
MPAIQPPLYLTFLLLTLLYLIITLYVWTILTINHNTAVRYAALYQRSFSRWGFDQSL

SEQ ID NO: 80 - protein, 57 aa, Mumps virus
MPAIQPPLYLTFLLLILLYLIITLYVWTILTINHKTAVRYAALYQRSCSRWGFDQSL

SEQ ID NO: 81 - protein, 57 aa, Mumps virus
MPAIQPPLYPTFLLLILLSLIITLYVWIISTITYKTAVRHAALYQRSFFRWSFDHSL

SEQ ID NO: 82 - protein, 57 aa, Mumps virus
MPAIQPPLHLTFLLLILLYLIITLYVWITLTITYKTAVRHATLYQRSFFRWSFDHPL

SEQ ID NO: 83 - protein, 57 aa, Mumps virus
MPAIQPPLYPTFLLLILLSLIITLYVWIISTITYKTAVRHASLYQRSFSRWSFDHSL

SEQ ID NO: 84 - protein, 57 aa, Mumps virus
MPAIQPPLYPTFLLLILLSLIITLYVWIISTITYKTAVRHAALHQRSFSRWSLDHSL

SEQ ID NO: 85 - protein, 15 aa, Artificial Sequence
  Genotype G Genbank ID QFO38283.1 1-15
MPAIQPPLYLTFLLL

SEQ ID NO: 86 - protein, 14 aa, Artificial Sequence
  Genotype G Genbank ID QFO38283.1 1-14
MPAIQPPLYLTFLL

SEQ ID NO: 87 - protein, 13 aa, Artificial Sequence
  Genotype G Genbank ID QFO38283.1 1-13
MPAIQPPLYLTFL

SEQ ID NO: 88 - protein, 12 aa, Artificial Sequence
  Genotype G Genbank ID QFO38283.1 1-12
MPAIQPPLYLTF

SEQ ID NO: 89 - protein, 11 aa, Artificial Sequence
  Genotype G Genbank ID QFO38283.1 1-11
MPAIQPPLYLT

SEQ ID NO: 90 - protein, 10 aa, Artificial Sequence
  Genotype G Genbank ID QFO38283.1 1-10
MPAIQPPLYL

SEQ ID NO: 91 - protein, 9 aa, Artificial Sequence
  Genotype G Genbank ID QFO38283.1 1-9
MPAIQPPLY

SEQ ID NO: 92 - protein, 14 aa, Artificial Sequence
  Genotype G Genbank ID QFO38283.1 2-15
PAIQPPLYLTFLLL

SEQ ID NO: 93 - protein, 13 aa, Artificial Sequence
  Genotype G Genbank ID QFO38283.1 2-14
PAIQPPLYLTFLL

SEQ ID NO: 94 - protein, 12 aa, Artificial Sequence
  Genotype G Genbank ID QFO38283.1 2-13
PAIQPPLYLTFL

SEQ ID NO: 95 - protein, 11 aa, Artificial Sequence
  Genotype G Genbank ID QFO38283.1 2-12
PAIQPPLYLTF

SEQ ID NO: 96 - protein, 10 aa, Artificial Sequence
  Genotype G Genbank ID QFO38283.1 2-11
PAIQPPLYLT

SEQ ID NO: 97 - protein, 9 aa, Artificial Sequence
  Genotype G Genbank ID QFO38283.1 2-10
PAIQPPLYL

SEQ ID NO: 98 - protein, 8 aa, Artificial Sequence
  Genotype G Genbank ID QFO38283.1 2-9
PAIQPPLY

SEQ ID NO: 99 - DNA, 20 bp, Artificial Sequence
  GPR125v2 Forward primer
tttctgactatgggcgaagg

SEQ ID NO: 100 - DNA, 20 bp, Artificial Sequence
  GPR125v2 Reverse primer
gcctccatgatggtgttctt

SEQ ID NO: 101 - DNA, 22 bp, Artificial Sequence
  GPR125pick Forward primer
atgcttgtgaacctgtgctttc

SEQ ID NO: 102 - DNA, 20 bp, Artificial Sequence
  GPR125pick Reverse primer
cgctggcatttctggtctgg

SEQ ID NO: 103 - DNA, 20 bp, Artificial Sequence
  YWHAZ Forward primer
agacggaaggtgctgagaaa

SEQ ID NO: 104 - DNA, 20 bp, Artificial Sequence
  YWHAZ Reverse primer
gaagcattggggatcaagaa

SEQ ID NO: 105 - DNA, 20 bp, Artificial Sequence
  clu Forward primer
atgaagattctcctgctgcg

SEQ ID NO: 106 - DNA, 19 bp, Artificial Sequence
  clu reverse primer
cttcaccaccacctcagtg

SEQ ID NO: 107 - DNA, 22 bp, Artificial Sequence
  Ttr Forward primer
agccctttgcctctgggaagac

SEQ ID NO: 108 - DNA, 22 bp, Artificial Sequence
  Ttr Reverse primer
tgcgatggtgtagtggcgatgg

SEQ ID NO: 109 - DNA, 20 bp, Artificial Sequence
  Ptgds Forward primer
gggaatcccaagagacccag

SEQ ID NO: 110 - DNA, 19 bp, Artificial Sequence
  Ptgds Reverse primer
gctctgagcaaatggctgc

SEQ ID NO: 111 - DNA, 27 bp, Artificial Sequence
  MMP-9 Forward primer
aaggacggccttctggcacacgccttt

SEQ ID NO: 112 - DNA, 24 bp, Artificial Sequence
  MMP-9 Reverse primer
gtggtatagtgggacacatagtgg

SEQ ID NO: 113 - DNA, 24 bp, Artificial Sequence
  occludin Forward primer
atgacttcaggcagcctcgttaca

SEQ ID NO: 114 - DNA, 24 bp, Artificial Sequence
  occludin Reverse primer
tcagcagcagccatgtactcttca

SEQ ID NO: 115 - protein, 57 aa, Artificial Sequence
  Genotype G Genbank ID QFO38283.1 1-57
  MISC_FEATURE (1)..(57): Genotype G Genbank ID QFO38283.1 1-57
MPAIQPPLYLTFLLLILLYLIITLYVWIILTITYKTAVRHAALYQRSFFRWSFDHSL

SEQ ID NO: 116 - protein, 15 aa, Artificial Sequence
  Genotype G Genbank ID QFO38283.1 2-16
  MISC_FEATURE (1)..(15): Genotype G Genbank ID QFO38283.1 2-16
PAIQPPLYLTFLLLI

SEQ ID NO: 117 - protein, 16 aa, Artificial Sequence
  Genotype G Genbank ID QFO38283.1 2-17
  MISC_FEATURE (1)..(16): Genotype G Genbank ID QFO38283.1 2-17
PAIQPPLYLTFLLLIL

SEQ ID NO: 118 - protein, 17 aa, Artificial Sequence
  Genotype G Genbank ID QFO38283.1 2-18
  MISC_FEATURE (1)..(17): Genotype G Genbank ID QFO38283.1 2-18
PAIQPPLYLTFLLLILL

SEQ ID NO: 119 - protein, 18 aa, Artificial Sequence
  Genotype G Genbank ID QFO38283.1 2-19
  MISC_FEATURE (1)..(18): Genotype G Genbank ID QFO38283.1 2-19
PAIQPPLYLTFLLLILLY

SEQ ID NO: 120 - protein, 19 aa, Artificial Sequence
  Genotype G Genbank ID QFO38283.1 2-20
  MISC_FEATURE (1)..(19): Genotype G Genbank ID QFO38283.1 2-20
PAIQPPLYLTFLLLILLYL

SEQ ID NO: 121 - protein, 20 aa, Artificial Sequence
  Genotype G Genbank ID QFO38283.1 2-21
  MISC_FEATURE (1)..(20): Genotype G Genbank ID QFO38283.1 2-21
PAIQPPLYLTFLLLILLYLI

SEQ ID NO: 122 - protein, 16 aa, Artificial Sequence
  Genotype G Genbank ID QFO38283.1 1-16
  MISC_FEATURE (1)..(16): Genotype G Genbank ID QFO38283.1 1-16
MPAIQPPLYLTFLLLI

SEQ ID NO: 123 - protein, 17 aa, Artificial Sequence
  Genotype G Genbank ID QFO38283.1 1-17
  MISC_FEATURE (1)..(17): Genotype G Genbank ID QFO38283.1 1-17
MPAIQPPLYLTFLLLIL

SEQ ID NO: 124 - protein, 18 aa, Artificial Sequence
  Genotype G Genbank ID QFO38283.1 1-18
  MISC_FEATURE (1)..(18): Genotype G Genbank ID QFO38283.1 1-18
MPAIQPPLYLTFLLLILL

SEQ ID NO: 125 - protein, 19 aa, Artificial Sequence
  Genotype G Genbank ID QFO38283.1 1-19
  MISC_FEATURE (1)..(19): Genotype G Genbank ID QFO38283.1 1-19
MPAIQPPLYLTFLLLILLY

SEQ ID NO: 126 - protein, 20 aa, Artificial Sequence
  Genotype G Genbank ID QFO38283.1 1-20
  MISC_FEATURE (1)..(20): Genotype G Genbank ID QFO38283.1 1-20
MPAIQPPLYLTFLLLILLYL

SEQ ID NO: 127 - protein, 30 aa, Artificial Sequence
  GLP-1 7-36
  MISC_FEATURE (1)..(30): GLP-1 7-36
HAEGTFTSDVSSYLEGQAAKEFIAWLVKGR

SEQ ID NO: 128 - protein, 30 aa, Artificial Sequence; identical to SEQ ID NO: 127
  SH(2-11) functionalized GLP-1(7-36)
  MISC_FEATURE (20)..(20): SH(2-11) (PAIQPPLYLT) protein connected via C-terminal residue and PEG2-spacer to Lys26 of GLP-1 (7-36)

SEQ ID NO: 129 - protein, 57 aa, Artificial Sequence; identical to SEQ ID NO: 74
  TAMRA-functionalized SH protein
  MISC_FEATURE (1)..(1): N-terminal TAMRA-functionalized SH-protein

SEQ ID NO: 130 - protein, 39 aa, Artificial Sequence
  Exendin-4 (1-39)
  MISC_FEATURE (1)..(39): Exendin-4 (1-39)
HGEGTFTSDLSKQMEEEAVRLFIEWLKNGGPSSGAPPPS

SEQ ID NO: 131 - protein, 39 aa, Artificial Sequence; identical to SEQ ID NO: 130
  SH(2-11) (PAIQPPLYLT) protein connected via C-terminal residue and PEG2-spacer to Lys 27 of Exendin-4 (1-39)
  MISC_FEATURE (27)..(27): SH(2-11) (PAIQPPLYLT) protein connected via C-terminal residue and PEG2-spacer to Lys27 of Exendin-4 (1-39)

SEQ ID NO: 132 - protein, 31 aa, Artificial Sequence
  GLP-1 7-37
  MISC_FEATURE (1)..(31): GLP-1 7-37
HAEGTFTSDVSSYLEGQAAKEFIAWLVKGRG

(19) United States
(12) Patent Application Publication
(10) Pub. No.: US 2024/0216519 A1
(43) Pub. Date: Jul. 4, 2024

(54) SMALL HYDROPHOBIC PROTEIN DRUG CONJUGATES AND USES THEREOF

(71) Applicants: UNIVERSITY OF COPENHAGEN, Copenhagen K (DK); DANMARKS TEKNISKE UNIVERSITET, Kongens Lyngby (DK)

(72) Inventors: Mette Marie ROSENKILDE, Hellerup (DK); Katja SPIESS, Humlebaek (DK); Lola Julie Torz PEDERSEN, Virum (DK); Maja Lind Nybo HENNINGSEN, Skibby (DK); Katrine QVORTRUP, Kgs. Lyngby (DK)

(21) Appl. No.: 18/556,402

(22) PCT Filed: Apr. 25, 2022
(86) PCT No.: PCT/EP2022/060884

§ 371 Date: Oct. 20, 2023

(30) Foreign Application Priority Data
Apr. 23, 2021 (EP) 21170192.5

(51) Int. Cl. A61K 47/64; A61K 38/16; A61K 38/26

(52) U.S. Cl. CPC A61K 47/64; A61K 38/162; A61K 38/26

(57) ABSTRACT

The present disclosure provides peptide conjugated drugs based on MuV SH Protein, their use as therapeutic agents and their use to provide delivery to and/or transfer across a membrane of the drug. The present disclosure provides use of the peptide conjugated drug for delivery of the drug to and/or across a membrane harbouring G protein-coupled receptor 125 (GPR125), such as the choroid plexus.

### TECHNICAL FIELD

The present invention provides peptide conjugated drugs, their use as therapeutic agents and their use to provide delivery to and/or transfer of the drug across a membrane. The present invention further provides treatment of diseases, such as diseases of the central nervous system by the use of the peptide conjugated drug.

### BACKGROUND

Targeted delivery of therapeutic agents to selected tissues and cell types is of high importance for treatment of various diseases. The targeting is especially difficult for treatment of diseases in organs, which are separated from and thereby protected from the general circulation by tight barriers, such as the central nervous system (CNS), the eye, and the testes. The barriers for entry into the CNS are divided into the blood-brain barriers (BBB), the arachnoid barrier, the blood spinal cord barrier, and the blood-cerebrospinal fluid barrier (BCSFB). In the eye, the blood-aqueous-barrier (BAB) is composed of tight junctions in the ciliary process non-pigmented epithelium, the endothelial cells in the iris vasculature, and the inner wall endothelium of Schlemm's canal. This barrier separates the inner of the eye from the blood (Coca-Prados, 2014).

The blood-testis barrier (BTB) is a barrier between the blood vessels and the seminiferous tubules. It is formed between sertoli cells in the seminiferous tubules (second layer of cells) located next to the stem cells (first/inner) layer of cells. It is also known as the Sertoli cell barrier (SCB). These protective barriers are of high importance since they serve to protect the vital compartments (brain, inner eye and testes) from various molecules and pathogens in the blood, with only selected molecules allowed to pass the barriers via different transport mechanisms. However, these barriers and their transport mechanism can also be targeted from a therapeutic point-of-view for selective and directed drug transport across the barriers. Many drug candidates, which would likely provide treatment of a given disease, if they could reach their respective molecular target in e.g. the CNS, have failed during the years due to difficulty in traversing these protective barriers to reach their desired molecular target. This issue remains a major obstacle in drug development to the CNS and may in many instances be the decisive factor for whether a potent compound becomes a successful drug in the end.

The BBB is a highly selective semipermeable membrane that prevents solutes in the circulating blood from non-selective crossing into the cerebrospinal fluid of the CNS. It is formed by tight junctions between the endothelial cells of brain capillaries. Passage of some molecules is allowed by passive diffusion, whereas selective transport is employed for various nutrients and macromolecules such as glucose, water and amino acids, which are crucial to neural function. In contrast, the BBB restricts the passage of e.g. pathogens and large or hydrophilic molecules into the brain.

The BCSFB, composed exclusively of the choroid plexus, is composed of tightly connected epithelial cells linked by tight junctions. It separates the blood outside the central nervous system (circulating blood) from the cerebrospinal fluid (CSF) in the ventricles. The blood-CSF boundary at the choroid plexus is composed of epithelial cells linked by tight junctions. CSF acts as a medium for the glymphatic filtration system that facilitates the removal of metabolic waste and the exchange of biomolecules into and out of the brain. Despite the similar function between the BBB and BCSFB, each facilitate the transport of different substances into the brain due to the distinct structural characteristics in the two barrier systems.

The BAB consists of two layers; an epithelium and an endothelial layer, both connected by tight junctions. The epithelium is either formed by the non-pigmented layer of the ciliary epithelium, or the posterior iridial epithelium, whereas the endothelium arise from the iridial vessels.

The BTB, composed of Sertoli cells with tight junctions, controls the adluminal environment in which germ cells develop by influencing the chemical composition of the luminal fluid and prevents passage of cytotoxic agents into the seminiferous tubules. The barrier also protects the germ cells from blood-borne harmful agents and prevents antigenic products of germ cell maturation from entering the circulation and generating an autoimmune response, which would ultimately result in reduced fertility of the sperm.

Overcoming the difficulty of delivering therapeutic agents to these compartments (eye and testes) and/or the brain presents a major challenge to treatment of various disorders.

Mechanisms for drug targeting in the brain have involved disruption by osmotic means, disruption biochemically by the use of vasoactive substances, or by localized exposure to high-intensity focused ultrasound (HIFU). Other methods used to get through the BBB have involved the use of endogenous transport systems, including carrier-mediated transporters, such as glucose and amino acid carriers, receptor-mediated transcytosis for insulin or transferrin, and the blocking of active efflux transporters.

Despite the above mentioned efforts in targeting of drugs to e.g. the brain by transport across the BBB, delivery of drugs and treatment of diseases in e,g, the CNS remain a challenge in drug development.

The G protein-coupled receptor (GPCR), GPR125, also called ADGRA3 (family GPCRs class B2), is highly expressed in cells and structures related to the three barriers mentioned above; BCSFB, BAB and BTB. Related to the BCSFB, GPR125 is highly expressed in the epithelial cells lining the choroid plexus (Petersen et al., 2020), the organ that is responsible for the production of cerebrospinal fluid (CSF) besides forming the barrier between the blood and the brain ventricles (BCSFB). GPR125 is also highly expressed in the inner layer of cells in the iris and in the ciliary body of the eye (Spina, 2021), thus expressed in the structures that are part of the BAB. In the testes, GPR125 is expressed in the spermatogonial stem cells (the inner/first layer of cells in the seminiferous tubules), and was early on described as a marker for these cells (He et al., 2012; Seandel et al., 2008; Xu et al., 2020). The barrier function between testes (seminiferous tubules) and the blood is formed by the second layer of cells, the sertoli cells, located in direct contact with the spermatogonial stem cells, thus again with a close and direct contact between GPR125 expression and a barrier (the BTB). In addition to these epithelia, GPR125 is expressed in other epithelia, such as the kidney and in the rest of the urogenital system (Seandel et al., 2008).

GPR125 is characterised by its 7 transmembrane (7TM) spanning domain that is conserved in all other adhesion receptors (aGPCRs), and resembles the well characterized class A GPCRs in its 7 transmembrane spanning alpha-helices. In addition to the 7TM domain (also denoted the C-terminal fragment, CTF) adhesion receptors are characterized by long extracellular N-termini that can contain adhesion or other functional domains (also denoted the N-terminal fragments, NTF).

GPR125 is an orphan receptor in a classical sense, as no endogenous extracellular ligands have been identified. When expressed in cultured cells, it undergoes constitutive clathrin-mediated arrestin-independent internalization (Spiess et al., 2019), however at present no signalling phenotype exists for GPR125. In zebrafish, GPR125 interacts with the cytoplasmic adaptor Dishevelled (Dvl) and recruits Frz7 and Glypican4 (Gpc4) complexes (Li et al., 2013). The intracellular parts of GPR125 also interact with Dlg (Large Disc protein of Drosophila) with a subsequent impact on the function of tight junctions (Woods et al., 1996). Moreover, recent studies have suggested a role in Wnt signalling, cell polarity, as well as in the repair of choroid plexus after injury (Li et al., 2013; Pickering et al., 2008). GPR125 was identified as interaction partner for the mumps virus encoded short hydrophobic (SH) protein (Woznik et al., 2010).

Mumps virus (MuV) used to be responsible for one of the most common infections in children (denoted mumps). While the number of infected patients has dropped significantly in the 80's due to the development of a vaccine, the virus was not eliminated. In the recent years, vaccine reputation has been questioned and an increasing number of persons resist being vaccinated, which led to an increase of mumps cases. MuV is a human pathogen and is highly neurotropic. After entering the bloodstream, MuV effectively travels to the brain, where it can cause meningitis and encephalitis. The MuV genome codes for seven genes expressing nine proteins, one of them is the short hydrophobic (SH) protein which is a membrane protein of 57 residues (6.8 KDa).

### SUMMARY

GPR125 is a G protein-coupled receptor highly expressed in the choroid plexus and other epithelia, such as the iris and the ciliary body in addition to the seminiferous tubules. The inventors of the present disclosure have identified that GPR125 is involved in membrane integrity and may be used as target for delivery of a composition across the membranes harbouring GPR125. The inventors have confirmed that the mumps virus short hydrophobic protein (MuV SH protein) is a ligand of GPR125, as previously disclosed (Woznik et al., 2010). Furthermore, the inventors have now identified that a short N-terminal fragment of MuV SH protein is capable of interacting with GPR125 on its own and that the interaction of MuV SH protein or an N-terminal fragment thereof with GPR125 results in reduced epithelial tightness. Further, conjugation of an N-terminal fragment of MuV SH-protein to peptide drugs based on GLP-1 or Exendin-4 increases their central effects (reducing food intake) indicating improved passage into the brain. Thus, the present invention relates to targeted delivery of a drug to and/or across a membrane or barrier harbouring GPR125 by conjugation of the drug to a MuV SH protein or a variant or fragment thereof as per the present disclosure, optionally via a linker.

In one aspect of the present disclosure a compound comprising a structure according to formula (I) is provided:

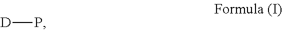

wherein
D comprises or consists of a drug; and;
P comprises or consists of a mumps virus small hydrophobic protein (MuV SH-protein) or a variant, a fragment, or a variant of a fragment thereof,
wherein D is conjugated to P, optionally via a linker L.

In one aspect of the present disclosure a compound comprising a structure according to formula (I) is provided for use in targeted delivery of D across a membrane or barrier harbouring GPR125 and/or to a site expressing GPR125, such as targeted delivery to the CNS, the eye or the testes.

In one aspect of the present disclosure a compound comprising a structure according to formula (I) is provided for use as a medicament.

In one aspect of the present disclosure a method for preparing a drug which is permeable to a barrier harbouring GPR125 is provided, such as permeable to the blood brain barrier, or the blood-cerebrospinal fluid barrier, the method comprising covalently conjugating the drug to MuV SH protein or a variant or a fragment, or a variant of a fragment thereof, optionally via a linker.

In one aspect of the present disclosure a method for delivering a drug across a membrane or barrier harbouring GPR125 is provided, such as delivering a drug across the choroid plexus, the method comprising providing the drug covalently conjugated to a MuV SH protein, optionally via a linker.

In one aspect of the present disclosure a method for targeted delivery of a drug to a cancer cell is provided, the method comprising providing the drug covalently conjugated to a MuV SH protein, optionally via a linker.

### DESCRIPTION OF DRAWINGS

FIG. 1. RNA expression of ADGRA1, 2 and 3 in wild-type (WT) vs. knocked down (KD) choroid plexus. RNA sequencing of choroid plexus isolated from six C57BL/6J mice which were bilaterally intracerebroventricular injected with either adeno associated virus (AAV) scramble shRNA (negative control (WT) three mice) or AAV shRNA GPR125 (to knock down GPR125 in the choroid plexus (KD) three mice). GPR125, (ADGRA3), is highly expressed in mouse choroid plexus of the WT mice and can be knocked down after AAV treatment. ADGRA1 (GPR123) is not expressed, or only expressed to a very low degree, in mouse choroid plexus. ADGRA2 (GPR124) is up regulated after GPR125 knock down and thereby, compensates the role of GPR125 for choroid plexus integrity. The data support the importance of the adhesion GPCRs GPR124 and GPR125 for the function of BCSFB.

FIG. 2. RNA sequencing of C57BL/6J mice injected with either AAV scramble shRNA (WT) or AAV shRNA GPR125 (GPR125 knock down) shows link between changes in GPR125 expression and extracellular structure and matrix organization related genes. The data of FIG. 2 is further provided in example 1, tables 3 to 5.

FIG. 3. RNA sequencing of C57BL/6J mice injected with either AAV scramble shRNA (WT) or AAV shRNA GPR125 (GPR125 knock down) shows link between changes in GPR125 expression and the morphogenesis of the epithelium and the regulation of transmembrane transport.

FIG. 4. A) Brain Ventricle Size by Dye-injection in wildtype or GPR125 knock out (KO) zebrafish. The width of the ventricles and the diameter of the eye and yoke sack were measured. We found no significant changes in the size of the ventricles and eye between WT and GPR125 KO zebrafish (n=7 in each group). The data of FIG. 4A is further provided in example 2, table 6. B) Permeability studies in wildtype or GPR125 knock out (KO) zebrafish. The micro injecting dextran conjugated dyes into blood stream of WT and GPR125 Knock out zebrafish at 28 hours post fertilization (hpf) revealed significantly higher signal of 3kDA dextran conjugated dye in the ventricles of the brain of the GPR125 KO fish compared to the WT zebrafish, confirming a leakiness of the Blood brain barriers in the brain of GPR125 KO zebrafish. (n=11 in each group). For the inner eye: P=0.0012; for the myencephalic ventricle: P=0,0126; for the diencephalic ventricle: P<0,0001, determined by two-way ANOVA followed by Bonferroni's multiple comparisons test.

FIG. 5. GPR125 expression levels after knock down and changes in gene expression of influenced genes determined by qPCR. A) Confirmation of decreased GPR125 expression levels (74%) in the choroid plexus of the AAV shRNA GPR125 infected C57BL/6J mice compared to WT mice (shRNA scramble; 100%) by qPCR. B) Knock down of GPR125 in mouse choroid plexus leads to down regulation of choroid plexus specific genes as the transporter transthyretin (Ttr). (n=3 per group).

FIG. 6. AAV scramble shRNA vs AAV shRNA GPR125 knock down in choroid plexus organoids (3D in vitro model). mCherry staining confirmed AAV entry into the organoids and expression of the scramble shRNA or shRNA GPR125. A) Organoid shows a normal 3D shape with cyst, while B) shows an organoid that has lost its 3D structure and is collapsed after treatment with AAV shRNA GPR125.

FIG. 7. Effect of mumps virus+/−SH protein and SH-protein ligands on the TransEpithelial Electric Resistance (TEER) of Caco2 cells. A) Caco2 cells were infected with mumps virus (MuV Jerryl Lynn strain, WT or SH-protein deletion virus) at a multiplicity of infection of 3. TEER was measured continuously performing CellZcope experiments. TEER was reduced at around 37 h post infection in Caco2 cells infected with the WT MuV compared to Caco2 cells infected with MuV SH-protein deletion virus. Representative results from three biological experiments (SEM), triplicates). B, C and D) Transport studies measuring the diffusion of the fluorescence dye (FITCs) after control (B: EDTA 6 mM) or ligands addition (C: SH-protein 2-15 aa (N-terminal truncation) and D: SH(2-11)-GLP-1(7-36) fusion protein through the tight monolayer of Caco2 cells. Results from one biological experiment with three technical replicates (SEM).

FIG. 8: TAMRA labelled SH-protein binds to choroid plexus of the WT mouse. Cryo-sections of mouse brains (WT and GPR125 KO mouse) were incubated for 1 h with the TAMRA-labelled SH-protein at different concentrations. The sections were mounted with mounting media containing a DAPI stain. A) The TAMRA labelled SH-protein binds to the choroid plexus of the WT mouse brain still at a concentration of 2,25 UM (visible as fine white line around the choroid plexus). B) The TAMRA labelled SH-protein binds to the choroid plexus of the GPR125 KO mouse brain only at higher concentrations of 22 μM and 11 μM (visible as a fine white line around the choroid plexus). The white staining from the choroid plexus is the DAPI staining and not staining from the TAMRA labelled SH-protein. The experiments were performed from two mouse brains WT and GPR125 KO respectively. Representative pictures presented.

FIG. 9. xCELLigence stimulation with SH protein fragments. The label-free, real-time xCELLigence technology is based on an impedance measurement of whole cells. Area under the curve (AUC) quantifications within the first 30 minutes after stimulation with SH protein fragments of varying size; SH1-9, SH2-9, SH2-15, and whole protein SH1-57. MDA231 cells endogenously express GPR125 (ADGRA3). HEK293 Flp-In T-rex cells have been stably transfected with full length (FL) or C-terminal fragment (CTF) GPR125 (ADGRA3). The data shown is n=2 for SH1-9, SH2-15, and SH1-57, whereas only a single experiment was carried out with SH2-9.

FIG. 10. The effect on food intake of fasted C57BL/6J mice injected with SH(2-11)-GLP-1(7-36) protein. Food intake was measured (A) 1 or (B) 2 hours after injection of either the positive control GLP-1(7-36) at 900 nmol/kg, GLP-1(7-36) at 200 nmol/kg, or SH(2-11)-GLP-1(7-36) protein (fusion protein of GLP-1 and N-terminus of the SH-protein (2-11)). ** , P<0.01 vs. vehicle, *** , P<0.005 vs. vehicle.

FIG. 11. cAMP accumulation by GLP-1(7-36) or SH(2-11)-GLP-1(7-36) protein on the (A) human or (B) rat GLP-1 receptor. Intracellular cAMP accumulation was measured in human (A) or rat (B) GLP-1R-expressing HEK293 cells after increasing amount of GLP-1(7-36) or SH(2-11)-GLP-1(7-36) protein (fusion protein of GLP-1 and the N-terminus of the SH-protein (2-11)). GLP-1(7-36) has an EC50 of 0,087 nM and 0,083 nM in human and rat GLP-1R, respectively. The SH(2-11)-GLP-1(7-36) fusion protein has an EC50 of 0.13 nM and 0.13 nM in the human and rat GLP-1R, respectively.

FIG. 12: Structure of SH(2-11)-GLP-1(7-36) conjugate.

FIG. 13: Structure of SH(2-11)-Exendin-4(1-39) conjugate.

FIG. 14. Effects of SH(2-11)-GLP-1(7-36) and GLP-1(7-36) on acute food intake in lean mice. Fasted lean C57BL/6J mice were injected subcutaneously right before dark phase with either vehicle, GLP-1(7-36) or SH(2-11)-GLP-1(7-36). One group served as positive controls of GLP-1(7-36) at 100 nmol/kg. Thirty minutes prior the peptide injection, all mice were administered with DDP-4 inhibitor Valine-pyrrolidide (1.5 mg/mouse). Food intake was measured after 1, 2, 4 and 14 hours. (A)-(D) Peptides dosed at 15 nmol/kg. SH(2-11)-GLP-1(7-36) significantly decreased food intake after 1, 2 and 4 hours compared to vehicle. *, P<0.05, ** , P<0.01, *** , P<0.0005. (E)-(H) Peptides dosed at 30 nmol/kg. Both GLP-1(7-36) and SH(2-11)-GLP-1(7-36) significantly decreased food intake after 1 and 2 hours compared to vehicle, however after 4 hours, food intake increased in GLP-1(7-36) treated mice compared to the SH(2-11)-GLP-1(7-36) group. The effect on food intake was still present after 14 hours in the SH(2-11)-GLP-1(7-36) group vs. vehicle. *, P<0.05, ** , P<0.005, *** P<0.001 and **** , P<0.0001.

FIG. 15. Effects of SH(2-11)-GLP-1(7-36) and GLP-1(7-36) on acute food intake in diet induced obese mice. Fasted C57BL/6NTac mice were injected subcutaneously right before dark phase with either vehicle, GLP-1(7-36) or SH(2-11)-GLP-1(7-36) at a dose of 15 nmol/kg. Thirty minutes prior injection, mice were administered with DDP-4 inhibitor Valine-pyrrolidide (3 mg/mouse). Food intake was measured after 1 (A), 2 (B), 4 (C) and 14 hours (D). SH(2-11)-GLP-1(7-36) significantly decreased food intake after 1, 2 and 4 hours compared to both vehicle and GLP-1(7-36). Food intake was also decreased in the SH(2-11)-GLP-1(7-36) group compared to vehicle after 14 hours. *, P<0.05, ** , P<0.01, *** , P=0.0005, **** , P<0.0001.

FIG. 16. Effects of SH(2-11)-Exendin-4(1-39) and Exendin-4(1-39) on acute food intake in lean mice. Lean C57BL/6J mice received a single injection subcutaneously of either vehicle, SH(2-11)-Exendin-4(1-39) or Exendin-4(1-39) at 15 nmol/kg right before dark phase. Food intake was measured after 1 (A), 2 (B), 4 (C) and 14 hours (D). SH(2-11)-Exendin-4(1-39) significantly decreased food intake after 1, 2 and 4 hours compared to both vehicle and Exendin-4(1-39). At 14 hours food intake was decreased in SH(2-11)-Exendin-4(1-39) treated mice compared to vehicle. *, P<0.05, ** P<0.01, *** P=0.0001, **** , P<0.0001.

### DETAILED DESCRIPTION

The present disclosure provides means for targeted delivery of a drug across a membrane or barrier harbouring GPR125, such as across the blood-cerebrospinal fluid barrier (BCSFB), the blood-aqueous-barrier (BAB), or the blood-testis barrier (BTB), and targeted delivery of a drug to a membrane or barrier harbouring GPR125. By conjugating the drug to a mumps virus small hydrophobic protein (MuV SH protein) or a fragment thereof, such as an N-terminal fragment thereof, the MuV SH protein will specifically interact with GPR125 to provide targeted delivery to the membrane or targeted delivery of the drug across the membrane or barrier.

G protein-coupled receptor 125 (GPR125, which is also known as ADGRA3, PGR21, TEM5L, and adhesion G protein-coupled receptor A3) is an orphan adhesion GPCR which was first identified in 2004. It is known to be expressed at high levels in e.g. the keratinocytes of the epidermis, in the neural cells of the cerebral cortex, in the choroid plexus epithelium and in the ovary and testes. Despite being known to be constitutively internalizing, its function has remained unknown. The present inventors have now demonstrated that GPR125 is involved in membrane integrity and that targeting of GPR125 provide targeted delivery of a drug to and/or across a membrane or barrier harbouring GPR125.

Due to the capability of MuV SH protein or a variant, a fragment or a variant of a fragment thereof (P) to interact with GPR125, the compounds of the present disclosure, provides targeted delivery of a drug (D) to and/or across a membrane or barrier harbouring GPR125, by conjugating said drug D to a MuV SH protein (P).

### Peptide Conjugated Drug

The present disclosure provides a peptide conjugated drug to facilitate targeted delivery to and/or across a membrane or barrier harbouring GPR125. A peptide conjugated drug may be referred to as a ‘compound’ herein, such as a compound comprising a drug (D) and a MuV SH protein (P), such as a compound comprising a drug (D) conjugated to a MuV SH protein (P).

In one embodiment, a compound is provided comprising a structure according to formula (I)

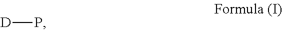

wherein
D comprises or consists of a drug; and
P comprises or consists of a mumps virus small hydrophobic protein (MuV SH protein); or a variant, a fragment, or a variant of a fragment thereof;
wherein D is conjugated to P, optionally via a linker L.

In one embodiment, D is covalently attached to P at the N-terminus of P, at the C-terminus of P or at an amino acid side chain of P. In one embodiment, D is covalently attached to P at the N-terminus of P. In one embodiment, D is covalently attached to P at the C-terminus of P. In one embodiment, D is covalently attached to P at an amino acid side chain of P.

In one embodiment, D is a protein- or peptide-based drug comprising a sequence of consecutive amino acids.

In one embodiment, P is covalently attached to D at the N-terminus of D, at the C-terminus of D or at an amino acid side chain of D. In one embodiment, P is covalently attached to D at the N-terminus of D. In one embodiment, P is covalently attached to D at the C-terminus of D. In one embodiment, P is covalently attached to D at an amino acid side chain of D.

In one embodiment, covalent attachment of D to P is a covalent attachment of D to P via a linker L.

In a preferred embodiment, D is covalently attached to P at the C-terminus of P, optionally via a linker L.

In one embodiment, D is covalently attached to P via a linker L.

In one embodiment, the compound comprises no linker, such as wherein D is directly covalently attached to P, without a linker.

In one embodiment, a compound is provided comprising a structure according to formula (II)

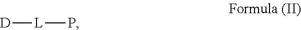

wherein
D comprises or consists of a drug;
P comprises or consists of a mumps virus small hydrophobic protein (MuV SH protein); or a variant, a fragment, or a variant of a fragment thereof; and
L is a linker;
   wherein D is conjugated to P via the linker L.

### Peptide ‘P’

The compound of the present disclosure comprises a drug D, which is conjugated to a peptide P, wherein P comprises or consists of a MuV SH protein, or a fragment thereof, a variant thereof, or a variant of a fragment thereof.

In one embodiment, P originates from any mumps virus strain and comprises or consists of any MuV SH protein or a fragment thereof, a variant thereof, or a variant of a fragment thereof.

In one embodiment, P originates from a clinical isolate or a patient isolate of mumps virus.

In one embodiment, P originates from a genotype A, B, C, D, F, G, H, I, J, K, L, or N mumps virus. In one embodiment, P originates from a genotype G mumps virus. In one embodiment, P originates from a MuV vaccine strain, such as originates from a Jeryl-Lynn strain, Urabe AM9 strain, Leningrad 3 strain, RIT 4385 strain, Leningrad-Zagreb strain, S₇₉ strain, Rubini strain, Hishino strain, RS(S-12) strain, Torii strain, or Miyahana strain.

In one embodiment, P originates from a genotype G or a vaccine strain mumps virus. Thus, in one embodiment, P comprises or consists of an amino acid sequence of MPAIX₁PPX₂X₃X₄ TFLLLX₅LLX₆LIX₇TLYVWX₈X₉X₁₀ X₁₁X₁₂X₁₃X₁₄X₁₅TX₁₆ VRX₁₇ AX₁₈LX₁₉QRSX₂₀X₂₁X₂₂ WX₂₃X₂₄DX₂₅X₂₆L (SEQ ID NO: 1); wherein:
X₁ is Q or R
X₂ is L or S
X₃ is Y or H
X₄ is L or P
X₅ is I or T
Xe is Y or S
X₇ is I, T or V
X₈ is I or T
X₉ is I or T
X₁₀ is L or S
X₁₁ is T or A
X₁₂ is V or I
X₁₃ is T or N
X₁₄ is Y or H
X₁₅ is K or N
X₁₆ is A or V
X₁₇ is H, P, Y or R
X₁₈ is A, T, or S
X₁₉ is Y or H
X₂₀ is F, C or S
X₂₁ is F, V or S
X₂₂ is H or R
X₂₃ is S, R or G
X₂₄ is F or L
X₂₅ is H or Q
X₂₆ is S, P, or T,
or a fragment thereof, a variant thereof, or a variant of a fragment thereof.

In one embodiment P comprises or consists of an amino acid sequence of MPAIX₁PPX₂X₃X₄ TFLLLX₅LLX₆LIX₇TLYVWX₈X₉X₁₀ X₁₁X₁₂X₁₃X₁₄X₁₅TX₁₆VRX₁₇ AX₁₈LX₁₉QRSX₂₀X₂₁X₂₂ WX₂₃X₂₄DX₂₅X₂₈L (SEQ ID NO: 1), or a fragment thereof, a variant thereof, or a variant of a fragment thereof,
wherein said fragment of P comprises at least amino acids 2-8 (PAIX₁PPX₂), such as comprises at least amino acids 2-9 (PAIX₁PPX₂X₃), and
wherein said variant of P comprises one or more amino acid substitutions, such as one amino acid substitution, such as two amino acid substitutions, such as three amino acid substitutions, such as four amino acid substitutions, such as five amino acid substitutions, and
wherein said variant of said fragment of P comprises at least amino acids 2-8 (PAIX₁PPX₂), such as comprises at least amino acids 2-9 (PAIX₁PPX₂X₃), and having one or more amino acid substitutions, such as one amino acid substitution, such as two amino acid substitutions, such as three amino acid substitutions, such as four amino acid substitutions, such as five amino acid substitutions within said fragment.

In one embodiment, P originates from a genotype G mumps virus. Thus, in one embodiment, P comprises or consists of an amino acid sequence of

PAIX₁PPX₂YLTFLLLILLYLIX₃TLYVWIILX₄VTYKTAVRX₅AALYQRSX₆X₇HWX₈FDHX₉L (SEQ ID NO: 2); wherein:
X₁ is Q or R;
X₂ is L or S;
X₃ is I or T;
X₄ is T or A;
X₅ is H, P or R;
X₆ is F or S;
X₇ is F or V;
X₈ is S or R; and
X₉ is S or T,
or a fragment thereof, a variant thereof, or a variant of a fragment thereof.

In one embodiment P comprises or consists of an amino acid sequence of PAIX1PPX₂YLTFLLLILLYLIX₃TLYVWIILX₄VTYKTAVRX₅AALYQRSX₆X₇HWX₈FDHX₉L (SEQ ID NO: 2), or a fragment thereof, a variant thereof, or a variant of a fragment thereof,
wherein said fragment of P comprises at least amino acids 1-8 (PAIX₁PPX₂Y), such as comprises at least amino acids 1-9 (PAIX₁PPX₂YL), and
wherein said variant of P comprises one or more amino acid substitutions, such as one amino acid substitution, such as two amino acid substitutions, such as three amino acid substitutions, such as four amino acid substitutions, such as five amino acid substitutions, and
wherein said variant of said fragment of P comprises at least amino acids 1-8 (PAIX₁PPX₂Y), such as comprises at least amino acids 1-9 (PAIX₁PPX₂YL), and having one or more amino acid substitutions, such as one amino acid substitution, such as two amino acid substitutions, such as three amino acid substitutions, such as four amino acid substitutions, such as five amino acid substitutions within said fragment

In one embodiment, P originates from a vaccine strain mumps virus. Thus, in one embodiment, P comprises or consists of an amino acid sequence of PAIQPPLX₁X₂ TFLLLX₃LLX₄LIX₅TLYVWX₆X₇X₈ TIX₉X₁₀X₁₁TX₁₂VRX₁₃ AX₁₄LX₁₅QRSX₁₆X₁₇RWX₁₈X₁₉DX₂₀X₂₁L (SEQ ID NO: 3); wherein:
X₁ is Y or H
X₂ is L or P
X₃ is I or T
X₄ is Y or S
X₅ is I or V
X₆ is I or T
X₇ is I or T
X₈ is L or S
X₉ is T or N
X₁₀ is Y or H
X₁₁ is K or N
X₁₂ is A or V
X₁₃ is Y or H
X₁₄ is A, T, or S
X₁₅ is H or Y
X₁₆ is F or C
X₁₇ is F or S
X₁₈ is S or G
X₁₉ is F or L
X₂₀ is H or Q, and
X₂₁ is S or P,
or a fragment thereof, a variant thereof, or a variant of a fragment thereof.

In one embodiment P comprises or consists of an amino acid sequence of PAIQPPLX₁X₂ TFLLLX₃LLX₄ LIX₅TLYVWX₆X₇X₈TIX₉X₁₀X₁₁ TX₁₂VRX₁₃ AX₁₄LX₁₅QRSX₁₆X₁₇R WX₁₈X₁₉DX₂₀X₂₁L (SEQ ID NO: 3), or a fragment thereof, a variant thereof, or a variant of a fragment thereof,
wherein said fragment of P comprises at least amino acids 1-8 (PAIQPPLX₁), such as comprises at least amino acids 1-9 (PAIQPPLX₁X₂), and
wherein said variant of P comprises one or more amino acid substitutions, such as one amino acid substitution, such as two amino acid substitutions, such as three amino acid substitutions, such as four amino acid substitutions, such as five amino acid substitutions, and
wherein said variant of said fragment of P comprises at least amino acids 1-8 (PAIQPPLX₁), such as comprises at least amino acids 1-9 (PAIQPPLX₁X₂), and having one or more amino acid substitutions, such as one amino acid substitution, such as two amino acid substitutions, such as three amino acid substitutions, such as four amino acid substitutions, such as five amino acid substitutions within said fragment.

In one embodiment P originates from Genotype G mumps virus strain Genbank ID QFO38283.1. Thus, in one embodiment, P comprises or consists of an amino acid sequence of PAIQPPLYLTFLLLILLYLIITLYVWIILTVTYKTAVRHAALYQRSFFHWSFDHSL (SEQ ID NO: 63), or a fragment thereof, a variant thereof, or a variant of a fragment thereof.

In one embodiment P comprises or consists of an amino acid sequence of PAIQPPLYLTFLLLILLYLIITLYVWIILTVTYKTAVRHAALYQRSFFHWSFDHSL (SEQ ID NO: 63), or a fragment thereof, a variant thereof, or a variant of a fragment thereof,
wherein said fragment of P comprises at least amino acids 1-8 (PAIQPPLY), such as comprises at least amino acids 2-9 (PAIQPPLYL), and
wherein said variant comprises one or more amino acid substitutions, such as one amino acid substitution, such as two amino acid substitutions, such as three amino acid substitutions, such as four amino acid substitutions, such as five amino acid substitutions, and
wherein said variant of said fragment comprises at least amino acids 1-8 (PAIQPPLY), such as comprises at least amino acids 2-9 (PAIQPPLYL), and having one or more amino acid substitutions, such as one amino acid substitution, such as two amino acid substitutions, such as three amino acid substitutions, such as four amino acid substitutions, such as five amino acid substitutions within said fragment.

In one embodiment, P originates from the Mumps virus Jeryl Lynn or RIT 4385 strain. Thus, in one embodiment, P comprises or consists of an amino acid sequence of PAIQPPLYLTFLLLTLLYLIITLYVWTILTINHNTAVRHAALYQRSFSRWGFDQSL (SEQ ID NO: 64), or a fragment thereof, a variant thereof, or a variant of a fragment thereof.

In one embodiment, P originates from the Mumps virus Urabi AM9 strain. Thus, in one embodiment, P comprises or consists of an amino acid sequence of PAIQPPLYP TFLLLILLSL IVTLYVWIIS TITYKTVVRH AALYQRSFFR WSFDHSL (SEQ ID NO: 65), or a fragment thereof, a variant thereof, or a variant of a fragment thereof.

In one embodiment, P originates from the Mumps virus Leningrad 3 strain. Thus, in one embodiment, P comprises or consists of an amino acid sequence of PAIQPPLYL TFLLLILLYL IITLYVWIIL TITYKTAVRH AALHQRSFFR WSFDHSL (SEQ ID NO: 66), or a fragment thereof, a variant thereof, or a variant of a fragment thereof.

In one embodiment, P originates from the Mumps virus Leningrad-Zagreb strain. Thus, in one embodiment, P comprises or consists of an amino acid sequence of PAIQPPLYL TELLLILLYL IITLYVWIIL TITYKTAVRH AALHQRSFFR WSFDHSL (SEQ ID NO: 67), or a fragment thereof, a variant thereof, or a variant of a fragment thereof.

In one embodiment, P originates from the Mumps virus S₇₉ strain. Thus, in one embodiment, P comprises or consists of an amino acid sequence of PAIQPPLYL TFLLLTLLYL IITLYVWTIL TINHNTAVRY AALYQRSFSR WGFDQSL (SEQ ID NO: 68), or a fragment thereof, a variant thereof, or a variant of a fragment thereof.

In one embodiment, P originates from the Mumps virus Rubini strain. Thus, in one embodiment, P comprises or consists of an amino acid sequence of PAIQPPLYL TFLLLILLYL IITLYVWTIL TINHKTAVRY AALYQRSCSR WGFDQSL (SEQ ID NO: 69), or a fragment thereof, a variant thereof, or a variant of a fragment thereof.

In one embodiment, P originates from the Mumps virus Hishino strain. Thus, in one embodiment, P comprises or consists of an amino acid sequence of PAIQPPLYP TFLLLILLSL IITLYVWIIS TITYKTAVRH AALYQRSFFR WSFDHSL (SEQ ID NO: 70), or a fragment thereof, a variant thereof, or a variant of a fragment thereof.

In one embodiment, P originates from the Mumps virus RS(S-12) strain. Thus, in one embodiment, P comprises or consists of an amino acid sequence of PAIQPPLHL TFLLLILLYL IITLYVWITL TITYKTAVRH ATLYQRSFFR WSFDHPL (SEQ ID NO: 71), or a fragment thereof, a variant thereof, or a variant of a fragment thereof.

In one embodiment, P originates from the Mumps virus Torii strain. Thus, in one embodiment, P comprises or consists of an amino acid sequence of PAIQPPLYP TFLLLILLSL IITLYVWIIS TITYKTAVRH ASLYQRSFSR WSFDHSL (SEQ ID NO: 72), or a fragment thereof, a variant thereof, or a variant of a fragment thereof.

In one embodiment, P originates from the Mumps virus Mijahada strain. Thus, in one embodiment, P comprises or consists of an amino acid sequence of PAIQPPLYP TFLLLILLSL IITLYVWIIS TITYKTAVRH AALHQRSFSR WSLDHSL (SEQ ID NO: 73), or a fragment thereof, a variant thereof, or a variant of a fragment thereof.

In one embodiment P comprises or consists of an amino acid sequence of a MuV SH protein 2-57 selected from the group consisting of SEQ ID NO: 63 to SEQ ID NO: 73, or a variant thereof, or a fragment thereof, or a variant of a fragment thereof.

In one embodiment, the amino acid sequence of P further comprises a methionine at the N-terminal end. Thus in one embodiment, P comprises or consists of an amino acid sequence selected from the group consisting of SEQ ID NO: 4, SEQ ID NO: 5, SEQ ID NO: 6, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, and a variant, a fragment, of a variant of a fragment of any one of SEQ ID NO: 4, SEQ ID NO: 5, SEQ ID NO: 6, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, or a variant thereof, or a fragment thereof, or a variant of a fragment thereof.

In one embodiment P comprises or consists of an amino acid sequence of a MuV SH protein 1-57 selected from the group consisting of SEQ ID NO: 74 to SEQ ID NO: 84, or a variant thereof, or a fragment thereof, or a variant of a fragment thereof.

As demonstrated herein, interaction of the MuV SH protein fragment with GPR125 results in reduced membrane integrity. Thus, in one embodiment, P is a fragment of a Muv SH protein, such as an N-terminal fragment of a MuV SH protein. In one embodiment, the drug D is conjugated to a fragment of a MuV SH protein, such as an N-terminal fragment of a MuV SH protein.

In one embodiment, P comprises or consists of a fragment of the MuV SH protein, such as comprises or consists of an N-terminal fragment of the MuV SH protein.

In one embodiment, P comprises or consists of a fragment of a MuV SH protein, such as comprises or consists of an N-terminal fragment of a MuV SH protein, wherein said MuV SH protein is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 63, SEQ ID NO: 64, SEQ ID NO: 65, SEQ ID NO: 66; SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83 and SEQ ID NO: 84.

In one embodiment, the present disclosure provides a compound of formula (I)

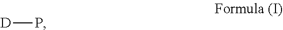

wherein
D comprises or consists of a drug; and
P is a fragment of a MuV SH protein, such as an N-terminal fragment of a MuV SH protein, wherein said MuV SH protein is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 63, SEQ ID NO: 64, SEQ ID NO: 65, SEQ ID NO: 66; SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83 and SEQ ID NO: 84; or a variant of said fragment,
   wherein D is conjugated to P, optionally via a linker L.

In one embodiment, P comprises or consists of a fragment of a MuV SH protein, in some embodiments having a sequence as disclosed herein elsewhere, having a length of less than 50 consecutive amino acid residues, for example less than 45 consecutive amino acid residues, such as less than 40 consecutive amino acid residues, for example less than 35 consecutive amino acid residues, such as less than 30 consecutive amino acid residues, for example less than 25 consecutive amino acid residues, such as less than 24 consecutive amino acid residues, for example less than 23 consecutive amino acid residues, such as less than 22 consecutive amino acid residues, for example less than 21 consecutive amino acid residues, such as less than 20 consecutive amino acid residues, for example less than 15 consecutive amino acid residues, such as less than 14 consecutive amino acid residues, for example less than 13 consecutive amino acid residues, such as less than 12 consecutive amino acid residues, for example less than 11 consecutive amino acid residues, such as less than 10 consecutive amino acid residues, for example less than 9 consecutive amino acid residues, such as less than 8 consecutive amino acid residues, for example less than 7 consecutive amino acid residues, such as less than 6 consecutive amino acid residues of said MuV SH protein.

In one embodiment, the amino acid sequence of the fragment of a MuV SH protein starts with amino acid 1 of said MuV SH protein. In one embodiment, the amino acid sequence of the fragment of a MuV SH protein starts with amino acid 1 of said MuV SH protein, which is methionine. In one embodiment, the amino acid sequence of the fragment of a MuV SH protein starts with amino acid 2 of said MuV SH protein. In one embodiment, the amino acid sequence of the fragment of a MuV SH protein starts with amino acid 2 of said MuV SH protein, i.e. excluding methionine.

In one embodiment, P comprises or consists of the 50 N-terminal consecutive amino acid residues of a MuV SH protein, in some embodiments having a sequence as disclosed herein elsewhere, for example the 45 N-terminal consecutive amino acid residues, such as the 40 N-terminal consecutive amino acid residues, for example comprises or consists of the 35 N-terminal consecutive amino acid residues, such as comprises or consists of the 30 N-terminal consecutive amino acid residues, for example comprises or consists of the 25 N-terminal consecutive amino acid residues, such as comprises or consists of the 24 N-terminal consecutive amino acid residues, for example comprises or consists of the 23 N-terminal consecutive amino acid residues, such as comprises or consists of the 22 N-terminal consecutive amino acid residues, for example comprises or consists of the 21 N-terminal consecutive amino acid residues, such as comprises or consists of the 20 N-terminal consecutive amino acid residues, for example comprises or consists of the 15 N-terminal consecutive amino acid residues, such as comprises or consists of the 14 N-terminal consecutive amino acid residues, for example comprises or consists of the 13 N-terminal consecutive amino acid residues, such as comprises or consists of the 12 N-terminal consecutive amino acid residues, for example comprises or consists of the 11 N-terminal consecutive amino acid residues, such as comprises or consists of the 10 N-terminal consecutive amino acid residues, for example comprises or consists of the 9 N-terminal consecutive amino acid residues, such as comprises or consists of the 8 N-terminal consecutive amino acid residues, for example comprises or consists of the 7 N-terminal consecutive amino acid residues, such as comprises or consists of the 6 N-terminal consecutive amino acid residues, for example comprises or consists of the 5 N-terminal consecutive amino acid residues of a MuV SH protein.

In one embodiment, P comprises or consists of the 50 N-terminal consecutive amino acid residues of SEQ ID NO: 1, for example the 45 N-terminal consecutive amino acid residues, such as the 40 N-terminal consecutive amino acid residues, for example comprises or consists of the 35 N-terminal consecutive amino acid residues, such as comprises or consists of the 30 N-terminal consecutive amino acid residues, for example comprises or consists of the 25 N-terminal consecutive amino acid residues, such as comprises or consists of the 24 N-terminal consecutive amino acid residues, for example comprises or consists of the 23 N-terminal consecutive amino acid residues, such as comprises or consists of the 22 N-terminal consecutive amino acid residues, for example comprises or consists of the 21 N-terminal consecutive amino acid residues, such as comprises or consists of the 20 N-terminal consecutive amino acid residues, for example comprises or consists of the 15 N-terminal consecutive amino acid residues, such as comprises or consists of the 14 N-terminal consecutive amino acid residues, for example comprises or consists of the 13 N-terminal consecutive amino acid residues, such as comprises or consists of the 12 N-terminal consecutive amino acid residues, for example comprises or consists of the 11 N-terminal consecutive amino acid residues, such as comprises or consists of the 10 N-terminal consecutive amino acid residues, for example comprises or consists of the 9 N-terminal consecutive amino acid residues, such as comprises or consists of the 8 N-terminal consecutive amino acid residues, for example comprises or consists of the 7 N-terminal consecutive amino acid residues, such as comprises or consists of the 6 N-terminal consecutive amino acid residues, for example comprises or consists of the 5 N-terminal consecutive amino acid residues of SEQ ID NO: 1.

In one embodiment, P comprises or consists of the 50 N-terminal consecutive amino acid residues of SEQ ID NO: 2, for example the 45 N-terminal consecutive amino acid residues, such as the 40 N-terminal consecutive amino acid residues, for example comprises or consists of the 35 N-terminal consecutive amino acid residues, such as comprises or consists of the 30 N-terminal consecutive amino acid residues, for example comprises or consists of the 25 N-terminal consecutive amino acid residues, such as comprises or consists of the 24 N-terminal consecutive amino acid residues, for example comprises or consists of the 23 N-terminal consecutive amino acid residues, such as comprises or consists of the 22 N-terminal consecutive amino acid residues, for example comprises or consists of the 21 N-terminal consecutive amino acid residues, such as comprises or consists of the 20 N-terminal consecutive amino acid residues, for example comprises or consists of the 15 N-terminal consecutive amino acid residues, such as comprises or consists of the 14 N-terminal consecutive amino acid residues, for example comprises or consists of the 13 N-terminal consecutive amino acid residues, such as comprises or consists of the 12 N-terminal consecutive amino acid residues, for example comprises or consists of the 11 N-terminal consecutive amino acid residues, such as comprises or consists of the 10 N-terminal consecutive amino acid residues, for example comprises or consists of the 9 N-terminal consecutive amino acid residues, such as comprises or consists of the 8 N-terminal consecutive amino acid residues, for example comprises or consists of the 7 N-terminal consecutive amino acid residues, such as comprises or consists of the 6 N-terminal consecutive amino acid residues, for example comprises or consists of the 5 N-terminal consecutive amino acid residues of SEQ ID NO: 2.

In one embodiment, P comprises or consists of the 50 N-terminal consecutive amino acid residues of SEQ ID NO: 3, for example the 45 N-terminal consecutive amino acid residues, such as the 40 N-terminal consecutive amino acid residues, for example comprises or consists of the 35 N-terminal consecutive amino acid residues, such as comprises or consists of the 30 N-terminal consecutive amino acid residues, for example comprises or consists of the 25 N-terminal consecutive amino acid residues, such as comprises or consists of the 24 N-terminal consecutive amino acid residues, for example comprises or consists of the 23 N-terminal consecutive amino acid residues, such as comprises or consists of the 22 N-terminal consecutive amino acid residues, for example comprises or consists of the 21 N-terminal consecutive amino acid residues, such as comprises or consists of the 20 N-terminal consecutive amino acid residues, for example comprises or consists of the 15 N-terminal consecutive amino acid residues, such as comprises or consists of the 14 N-terminal consecutive amino acid residues, for example comprises or consists of the 13 N-terminal consecutive amino acid residues, such as comprises or consists of the 12 N-terminal consecutive amino acid residues, for example comprises or consists of the 11 N-terminal consecutive amino acid residues, such as comprises or consists of the 10 N-terminal consecutive amino acid residues, for example comprises or consists of the 9 N-terminal consecutive amino acid residues, such as comprises or consists of the 8 N-terminal consecutive amino acid residues, for example comprises or consists of the 7 N-terminal consecutive amino acid residues, such as comprises or consists of the 6 N-terminal consecutive amino acid residues, for example comprises or consists of the 5 N-terminal consecutive amino acid residues of SEQ ID NO: 3.

In one embodiment, P comprises or consists of the 50 N-terminal consecutive amino acid residues of SEQ ID NO: 4, for example the 45 N-terminal consecutive amino acid residues, such as the 40 N-terminal consecutive amino acid residues, for example comprises or consists of the 35 N-terminal consecutive amino acid residues, such as comprises or consists of the 30 N-terminal consecutive amino acid residues, for example comprises or consists of the 25 N-terminal consecutive amino acid residues, such as comprises or consists of the 24 N-terminal consecutive amino acid residues, for example comprises or consists of the 23 N-terminal consecutive amino acid residues, such as comprises or consists of the 22 N-terminal consecutive amino acid residues, for example comprises or consists of the 21 N-terminal consecutive amino acid residues, such as comprises or consists of the 20 N-terminal consecutive amino acid residues, for example comprises or consists of the 15 N-terminal consecutive amino acid residues, such as comprises or consists of the 14 N-terminal consecutive amino acid residues, for example comprises or consists of the 13 N-terminal consecutive amino acid residues, such as comprises or consists of the 12 N-terminal consecutive amino acid residues, for example comprises or consists of the 11 N-terminal consecutive amino acid residues, such as comprises or consists of the 10 N-terminal consecutive amino acid residues, for example comprises or consists of the 9 N-terminal consecutive amino acid residues, such as comprises or consists of the 8 N-terminal consecutive amino acid residues, for example comprises or consists of the 7 N-terminal consecutive amino acid residues, such as comprises or consists of the 6 N-terminal consecutive amino acid residues, for example comprises or consists of the 5 N-terminal consecutive amino acid residues of SEQ ID NO: 4.

In one embodiment, P comprises or consists of the 50 N-terminal consecutive amino acid residues of SEQ ID NO: 5, for example the 45 N-terminal consecutive amino acid residues, such as the 40 N-terminal consecutive amino acid residues, for example comprises or consists of the 35 N-terminal consecutive amino acid residues, such as comprises or consists of the 30 N-terminal consecutive amino acid residues, for example comprises or consists of the 25 N-terminal consecutive amino acid residues, such as comprises or consists of the 24 N-terminal consecutive amino acid residues, for example comprises or consists of the 23 N-terminal consecutive amino acid residues, such as comprises or consists of the 22 N-terminal consecutive amino acid residues, for example comprises or consists of the 21 N-terminal consecutive amino acid residues, such as comprises or consists of the 20 N-terminal consecutive amino acid residues, for example comprises or consists of the 15 N-terminal consecutive amino acid residues, such as comprises or consists of the 14 N-terminal consecutive amino acid residues, for example comprises or consists of the 13 N-terminal consecutive amino acid residues, such as comprises or consists of the 12 N-terminal consecutive amino acid residues, for example comprises or consists of the 11 N-terminal consecutive amino acid residues, such as comprises or consists of the 10 N-terminal consecutive amino acid residues, for example comprises or consists of the 9 N-terminal consecutive amino acid residues, such as comprises or consists of the 8 N-terminal consecutive amino acid residues, for example comprises or consists of the 7 N-terminal consecutive amino acid residues, such as comprises or consists of the 6 N-terminal consecutive amino acid residues, for example comprises or consists of the 5 N-terminal consecutive amino acid residues of SEQ ID NO: 5.

In one embodiment, P comprises or consists of the 50 N-terminal consecutive amino acid residues of SEQ ID NO: 6, for example the 45 N-terminal consecutive amino acid residues, such as the 40 N-terminal consecutive amino acid residues, for example comprises or consists of the 35 N-terminal consecutive amino acid residues, such as comprises or consists of the 30 N-terminal consecutive amino acid residues, for example comprises or consists of the 25 N-terminal consecutive amino acid residues, such as comprises or consists of the 24 N-terminal consecutive amino acid residues, for example comprises or consists of the 23 N-terminal consecutive amino acid residues, such as comprises or consists of the 22 N-terminal consecutive amino acid residues, for example comprises or consists of the 21 N-terminal consecutive amino acid residues, such as comprises or consists of the 20 N-terminal consecutive amino acid residues, for example comprises or consists of the 15 N-terminal consecutive amino acid residues, such as comprises or consists of the 14 N-terminal consecutive amino acid residues, for example comprises or consists of the 13 N-terminal consecutive amino acid residues, such as comprises or consists of the 12 N-terminal consecutive amino acid residues, for example comprises or consists of the 11 N-terminal consecutive amino acid residues, such as comprises or consists of the 10 N-terminal consecutive amino acid residues, for example comprises or consists of the 9 N-terminal consecutive amino acid residues, such as comprises or consists of the 8 N-terminal consecutive amino acid residues, for example comprises or consists of the 7 N-terminal consecutive amino acid residues, such as comprises or consists of the 6 N-terminal consecutive amino acid residues, for example comprises or consists of the 5 N-terminal consecutive amino acid residues of SEQ ID NO: 6.

In one embodiment, P comprises or consists of the 50 N-terminal consecutive amino acid residues of a MuV SH protein selected from the group consisting of SEQ ID NO: 63, SEQ ID NO: 64, SEQ ID NO: 65, SEQ ID NO: 66; SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83 and SEQ ID NO: 84, for example the 45 N-terminal consecutive amino acid residues, such as the 40 N-terminal consecutive amino acid residues, for example comprises or consists of the 35 N-terminal consecutive amino acid residues, such as comprises or consists of the 30 N-terminal consecutive amino acid residues, for example comprises or consists of the 25 N-terminal consecutive amino acid residues, such as comprises or consists of the 24 N-terminal consecutive amino acid residues, for example comprises or consists of the 23 N-terminal consecutive amino acid residues, such as comprises or consists of the 22 N-terminal consecutive amino acid residues, for example comprises or consists of the 21 N-terminal consecutive amino acid residues, such as comprises or consists of the 20 N-terminal consecutive amino acid residues, for example comprises or consists of the 15 N-terminal consecutive amino acid residues, such as comprises or consists of the 14 N-terminal consecutive amino acid residues, for example comprises or consists of the 13 N-terminal consecutive amino acid residues, such as comprises or consists of the 12 N-terminal consecutive amino acid residues, for example comprises or consists of the 11 N-terminal consecutive amino acid residues, such as comprises or consists of the 10 N-terminal consecutive amino acid residues, for example comprises or consists of the 9 N-terminal consecutive amino acid residues, such as comprises or consists of the 8 N-terminal consecutive amino acid residues, for example comprises or consists of the 7 N-terminal consecutive amino acid residues, such as comprises or consists of the 6 N-terminal consecutive amino acid residues, for example comprises or consists of the 5 N-terminal consecutive amino acid residues of a MuV SH protein selected from the group consisting of SEQ ID NO: 63, SEQ ID NO: 64, SEQ ID NO: 65, SEQ ID NO: 66; SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83 and SEQ ID NO: 84.

In a preferred embodiment, P comprises or consists of 15 or less than the 15 N-terminal consecutive amino acid residues of a MuV SH protein, in some embodiments of a MuV SH protein having a sequence as disclosed herein elsewhere. In one embodiment, P comprises or consists of less than the 15 N-terminal consecutive amino acid residues of SEQ ID NO: 1. In one embodiment, P comprises or consists of less than the 15 N-terminal consecutive amino acid residues of SEQ ID NO: 2. In one embodiment, P comprises or consists of less than the 15 N-terminal consecutive amino acid residues of SEQ ID NO: 3. In one embodiment, P comprises or consists of less than the 15 N-terminal consecutive amino acid residues of SEQ ID NO: 4. In one embodiment, P comprises or consists of less than the 15 N-terminal consecutive amino acid residues of SEQ ID NO: 5. In one embodiment, P comprises or consists of less than the 15 N-terminal consecutive amino acid residues of SEQ ID NO: 6. In one embodiment, P comprises or consists of less than the 15 N-terminal consecutive amino acid residues of any one of SEQ ID NO: 63 to SEQ ID NO: 84.

In one embodiment, the present disclosure provides a compound of formula (I)

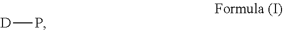

wherein
D comprises or consists of a drug; and
P is an N-terminal fragment comprising or consisting of the 5 to 14 N-terminal consecutive amino acid residues of
wherein said MuV SH protein is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 63, SEQ ID NO: 64, SEQ ID NO: 65, SEQ ID NO: 66; SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83 and SEQ ID NO: 84; or a variant of said fragment,
   wherein D is conjugated to P, optionally via a linker L.

In one embodiment, P comprises or consists of in the range of the 5 to 14 N-terminal consecutive amino acid residues of a MuV SH protein, such as in the range of the 5-6, 6-7, 7-8, 8-9, 9-10, 10-11, 11-12, 12-13, or 13-14 N-terminal consecutive amino acid residues of a MuV SH protein; in some embodiments of a MuV SH protein having a sequence as disclosed herein elsewhere.

In one embodiment, P comprises or consists of in the range of the 5 to 14 N-terminal consecutive amino acid residues of SEQ ID NO: 1, such in the range of the 5-6, 6-7, 7-8, 8-9, 9-10, 10-11, 11-12, 12-13, or 13-14 N-terminal consecutive amino acid residues of SEQ ID NO: 1. In one embodiment, P comprises or consists of in the range of the 5 to 14 N-terminal consecutive amino acid residues of SEQ ID NO: 2, such in the range of the 5-6, 6-7, 7-8, 8-9, 9-10, 10-11, 11-12, 12-13, or 13-14 N-terminal consecutive amino acid residues of SEQ ID NO: 2. In one embodiment, P comprises or consists of in the range of the 5 to 14 N-terminal consecutive amino acid residues of SEQ ID NO: 3, such in the range of the 5-6, 6-7, 7-8, 8-9, 9-10, 10-11, 11-12, 12-13, or 13-14 N-terminal consecutive amino acid residues of SEQ ID NO: 3. In one embodiment, P comprises or consists of in the range of the 5 to 14 N-terminal consecutive amino acid residues of SEQ ID NO: 4, such in the range of the 5-6, 6-7, 7-8, 8-9, 9-10, 10-11, 11-12, 12-13, or 13-14 N-terminal consecutive amino acid residues of SEQ ID NO: 4. In one embodiment, P comprises or consists of in the range of the 5 to 14 N-terminal consecutive amino acid residues of SEQ ID NO: 5, such in the range of the 5-6, 6-7, 7-8, 8-9, 9-10, 10-11, 11-12, 12-13, or 13-14 N-terminal consecutive amino acid residues of SEQ ID NO: 5. In one embodiment, P comprises or consists of in the range of the 5 to 14 N-terminal consecutive amino acid residues of SEQ ID NO: 6, such in the range of the 5-6, 6-7, 7-8, 8-9, 9-10, 10-11, 11-12, 12-13, or 13-14 N-terminal consecutive amino acid residues of SEQ ID NO: 6. In one embodiment, P comprises or consists of in the range of the 5 to 14 N-terminal consecutive amino acid residues of any one of SEQ ID NO: 63 to SEQ ID NO: 84, such in the range of the 5-6, 6-7, 7-8, 8-9, 9-10, 10-11, 11-12, 12-13, or 13-14 N-terminal consecutive amino acid residues of any one of SEQ ID NO: 63 to SEQ ID NO: 84.

In a preferred embodiment, P comprises or consists of the 8 or the 9 N-terminal consecutive amino acid residues of a MuV SH protein, in some embodiments of a MuV SH protein having a sequence as disclosed herein elsewhere. In a preferred embodiment, P comprises or consists of amino acid residues 1 to 8 or 1 to 9 of a MuV SH protein in some embodiments of a MuV SH protein having a sequence as disclosed herein elsewhere.

In one embodiment, P comprises or consists of the 8 N-terminal consecutive amino acid residues of SEQ ID NO: 1. In one embodiment, P comprises or consists of the 8 N-terminal consecutive amino acid residues of SEQ ID NO: 2. In one embodiment, P comprises or consists of the 8 N-terminal consecutive amino acid residues of SEQ ID NO: 3. In one embodiment, P comprises or consists of the 9 N-terminal consecutive amino acid residues of SEQ ID NO: 4. In one embodiment, P comprises or consists of the 9 N-terminal consecutive amino acid residues of SEQ ID NO: 5. In one embodiment, P comprises or consists of the 9 N-terminal consecutive amino acid residues of SEQ ID NO: 6.

In one embodiment, P comprises or consists of the 8 N-terminal consecutive amino acid residues of any one of SEQ ID NO: 1 to 3 (MuV SH protein 2-9).

In one embodiment, P comprises or consists of the 8 N-terminal consecutive amino acid residues of any one of SEQ ID NO: 63 to 73 (MuV SH protein 2-9).

In one embodiment, P comprises or consists of the 9 N-terminal consecutive amino acid residues of any one of SEQ ID NO: 4 to 6 (MuV SH protein 1-9). In one embodiment, P comprises or consists of the 9 N-terminal consecutive amino acid residues of any one of SEQ ID NO: 74 to 84 (MuV SH protein 1-9).

In one embodiment, P comprises or consists of an amino acid sequence selected from the group consisting of MPAIX₁PPX₂X₃X₄ TFLLL (SEQ ID NO: 7), MPAIX₁PPX₂X₃X₄ TFLL (SEQ ID NO: 10), MPAIX₁PPX₂X₃X₄ TFL (SEQ ID NO: 13), MPAIX₁PPX₂X₃X₄ TF (SEQ ID NO: 16), MPAIX₁PPX₂X₃X₄ T (SEQ ID NO: 19), MPAIX₁PPX₂X₃X₄ (SEQ ID NO: 22), MPAIX₁PPX₂X₃ (SEQ ID NO: 25), MPAIX₁PPX₂ (SEQ ID NO: 27), MPAIX₁PP (SEQ ID NO: 29), MPAIX₁P (SEQ ID NO: 31), MPAIX₁ (SEQ ID NO: 33), PAIX₁PPX₂X₃X₄ TFLLL (SEQ ID NO: 35), PAIX₁PPX₂X₃X₄ TFLL (SEQ ID NO: 38), PAIX₁PPX₂X₃X₄ TFL (SEQ ID NO: 41), PAIX₁PPX₂X₃X₄ TF (SEQ ID NO: 44), PAIX₁PPX₂X₃X₄ T (SEQ ID NO: 47), PAIX₁PPX₂X₃X₄ (SEQ ID NO: 50), PAIX₁PPX₂X₃ (SEQ ID NO: 53), PAIX₁PPX₂ (SEQ ID NO: 55), PAIX₁PP (SEQ ID NO: 57), PAIX₁P (SEQ ID NO: 59), and PAIX₁ (SEQ ID NO: 61), wherein:
X₁ is Q or R
X₂ is L or S
X₃ is Y or H, and
X₄ is L or P.

In one embodiment, P comprises or consists of an amino acid sequence selected from the group consisting of MPAIX₁PPX₂YL TFLLL (SEQ ID NO: 8), MPAIX₁PPX₂YL TFLL (SEQ ID NO: 11), MPAIX₁PPX₂YL TFL (SEQ ID NO: 14), MPAIX₁PPX₂YL TF (SEQ ID NO: 17), MPAIX₁PPX₂YL T (SEQ ID NO: 20), MPAIX₁PPX₂YL (SEQ ID NO: 23), MPAIX₁PPX₂Y (SEQ ID NO: 25), MPAIX₁PPX₂ (SEQ ID NO: 27), MPAIX₁PP (SEQ ID NO: 29), MPAIX₁P (SEQ ID NO: 31), MPAIX₁ (SEQ ID NO: 33), PAIX₁PPX₂YL TFLLL (SEQ ID NO: 36), PAIX₁PPX₂YL TELL (SEQ ID NO: 39), PAIX₁PPX₂YL TFL (SEQ ID NO: 42), PAIX₁PPX₂YL TF (SEQ ID NO: 45), PAIX₁PPX₂YL T (SEQ ID NO: 48), PAIX₁PPX₂YL (SEQ ID NO: 51), PAIX₁PPX₂Y (SEQ ID NO: 53), PAIX₁PPX₂ (SEQ ID NO: 55), PAIX₁PP (SEQ ID NO: 57), PAIX₁P (SEQ ID NO: 59), MPAIX₁ (SEQ ID NO: 61), wherein:
X₁ is Q or R, and
X₂ is L or S.

In one embodiment, P comprises or consists of an amino acid sequence selected from the group consisting of MPAIQPPLX₁X₂ TFLLL (SEQ ID NO: 9), MPAIQPPLX₁X₂ TELL (SEQ ID NO: 12), MPAIQPPLX₁X₂ TFL (SEQ ID NO: 15), MPAIQPPLX₁X₂ TF (SEQ ID NO: 18), MPAIQPPLX₁X₂ T (SEQ ID NO: 21), MPAIQPPLX₁X₂ (SEQ ID NO: 24), MPAIQPPLX₁ (SEQ ID NO: 26), MPAIQPPL (SEQ ID NO: 28), MPAIQPP (SEQ ID NO: 30), MPAIQP (SEQ ID NO: 32), MPAIQ (SEQ ID NO: 34), PAIQPPLX₁X₂ TFLLL (SEQ ID NO: 37), PAIQPPLX₁X₂ TFLL (SEQ ID NO: 40), PAIQPPLX₁X₂ TFL (SEQ ID NO: 43), PAIQPPLX₁X₂ TF (SEQ ID NO: 46), PAIQPPLX X₂ T (SEQ ID NO: 49), PAIQPPLX₁X₂ (SEQ ID NO: 52), PAIQPPLX₁ (SEQ ID NO: 54), PAIQPPL (SEQ ID NO: 56), PAIQPP (SEQ ID NO: 58), PAIQP (SEQ ID NO: 60), PAIQ (SEQ ID NO: 62), wherein:
X₁ is Y or H, and
X₂ is L or P.

In one embodiment, P comprises of consists of an amino acid sequence selected from the group consisting of MPAIQPPLYL TFLLL (SEQ ID NO: 85), MPAIQPPLYL TFLL (SEQ ID NO: 86), MPAIQPPLYL TFL (SEQ ID NO: 87), MPAIQPPLYL TF (SEQ ID NO: 88), MPAIQPPLYL T (SEQ ID NO: 89), MPAIQPPLYL (SEQ ID NO: 90), MPAIQPPLY (SEQ ID NO: 91), MPAIQPPL (SEQ ID NO: 28), MPAIQPP (SEQ ID NO: 30), MPAIQP (SEQ ID NO: 32), MPAIQ (SEQ ID NO: 34), PAIQPPLYL TFLLL (SEQ ID NO: 92), PAIQPPLYL TELL (SEQ ID NO: 93, PAIQPPLYL TFL (SEQ ID NO: 94), PAIQPPLYL TF (SEQ ID NO: 95), PAIQPPLYL T (SEQ ID NO: 96), PAIQPPLYL (SEQ ID NO: 97), PAIQPPLY (SEQ ID NO: 98), PAIQPPL (SEQ ID NO: 56), PAIQPP (SEQ ID NO: 58), PAIQP (SEQ ID NO: 60), and PAIQ (SEQ ID NO: 62), or a variant thereof.

In one embodiment, the present disclosure provides a compound of formula (I)

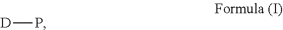

wherein
D comprises or consists of a drug; and
P comprises of consists of an amino acid sequence selected from the group consisting SEQ ID NO: 7 to SEQ ID NO:62 and SEQ ID NO:85 to SEQ ID NO: 98, or a variant thereof, wherein said variant comprises one or more amino acid substitutions,
   wherein D is conjugated to P, optionally via a linker L.

In one embodiment, P comprises of consists of an amino acid sequence selected from the group consisting of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, and SEQ ID NO: 62, or a variant thereof.

In one embodiment, P comprises of consists of an amino acid sequence selected from the group consisting of SEQ ID NO: 7 to SEQ ID NO:62 and SEQ ID NO:85 to SEQ ID NO: 98, or a variant thereof, wherein said variant comprises one or more amino acid substitutions.

In one embodiment, P comprises of consists of an amino acid sequence selected from the group consisting of SEQ ID NO: 7 to SEQ ID NO:62 and SEQ ID NO:85 to SEQ ID NO: 98, or a variant thereof, wherein said variant comprises one amino acid substitution, such as two amino acid substitutions, such as three amino acid substitutions, such as four amino acid substitutions.

In one embodiment, P is a variant of a MuV SH protein, in some embodiments of a MuV SH protein having a sequence as disclosed herein elsewhere. In one embodiment, the variant comprises an amino acid substitution, an amino acid deletion, and/or an amino acid insertion. In one embodiment, the amino acid substitution is a conservative amino acid substitution, such as an amino acid substitution not affecting the structure or function of the MuV SH protein.

In one embodiment, the variant of P comprises less than 10 amino acid substitutions, deletions and/or insertions, such as less than 9 amino acid substitutions, deletions and/or insertions, such as less than 8 amino acid substitutions, deletions and/or insertions, such as less than 7 amino acid substitutions, deletions and/or insertions, such as less than 6 amino acid substitutions, deletions and/or insertions, such as less than 5 amino acid substitutions, deletions and/or insertions, such as less than 4 amino acid substitutions, deletions and/or insertions, such as less than 3 amino acid substitutions, deletions and/or insertions, such as less than 2 amino acid substitutions, deletions and/or insertions, such as less than 1 amino acid substitutions, deletions and/or insertions.

In one embodiment, P is a variant of a fragment of a MuV SH protein, in some embodiments of a MuV SH protein having a sequence as disclosed herein elsewhere. In one embodiment, the variant comprises an amino acid substitution, an amino acid deletion, and/or an amino acid insertion. In one embodiment, the amino acid substitution is a conservative amino acid substitution, such as an amino acid substitution not affecting the structure or function of the fragment of the MuV SH protein. In one embodiment, the variant of the fragment of P comprises less than 5 amino acid substitutions, deletions and/or insertions, such as less than 4 amino acid substitutions, deletions and/or insertions, such as less than 3 amino acid substitutions, deletions and/or insertions, such as less than 2 amino acid substitutions, deletions and/or insertions, such as less than 1 amino acid substitutions, deletions and/or insertions.

In one embodiment, P is a variant of a fragment of a MuV SH protein, in some embodiments of a MuV SH protein having a sequence as disclosed herein elsewhere.

In one embodiment, P is a variant of a MuV SH protein, such as a MuV SH protein fragment according to the present disclosure. In one embodiment said variant comprises one amino acid substitution, such as two amino acid substitutions, such as three amino acid substitutions, such as 4 amino acid substitutions, such as 5 amino acid substitutions, such as 6 amino acid substitutions, such as 7 amino acid substitutions, such as 8 amino acid substitutions.

In one embodiment, P is capable of interacting with GPR125, such as capable of binding to GPR125. In one embodiment, P is capable of acting as an antagonist of GPR125, such as capable of inducing the same effect as when GPR125 is not present, such as inducing the same or similar effect as observed by knock-out of GPR125. In one embodiment, interaction of P with GPR125 result in reduced membrane integrity. A reduced membrane integrity may be measured as a reduction in TransEpithelial Electric Resistance (TEER) as described in example 4. In one embodiment, interaction of P with GPR125 result in reduced TransEpithelial Electric Resistance (TEER). In one embodiment, reduced membrane integrity leads to increased permeability of the membrane whereby D will be able to cross the membrane, such as be able to enter the ventricle and subsequent delivery to it targets by CSF diffusion. The increase permeability can be measured as an increased effect of D as it reaches its primary target in the brain or an increased concentration of D at the site of delivery. In one embodiment, interaction of P with GPR125 result in internalization of P including the drug conjugated to P.

### Peptide P Fragments and Variants

The term “fragment of peptide P” and “peptide P fragment” is to be understood as a part, portion or subsequence of the peptide P, wherein P is a MuV SH protein.

In one embodiment, P is a N-terminal fragment of a MuV SH protein. In one embodiment, P is a N-terminal fragment of the MuV SH protein as set forth in any one of SEQ ID NO: 63-73. In one embodiment, P is a N-terminal fragment of the MuV SH protein as set forth in any one of SEQ ID NO: 74-84. In one embodiment, P is a N-terminal fragment of the MuV SH protein as set forth in any one of SEQ ID NO: 74, 77 or 78. In one embodiment, P is a N-terminal fragment of a MuV SH protein as set forth in SEQ ID NO: 115 (SH1-57).

In one embodiment, P comprises the sequence PAIQPPLY (SEQ ID NO: 98). In one embodiment, P comprises the sequence PAIQPPLY (SEQ ID NO: 98) and comprises or consists of in the range of the 5 to 20 consecutive amino acid residues of a MuV SH protein, such as in the range of the 5-6, 6-7, 7-8, 8-9, 9-10, 10-11, 11-12, 12-13, 13-14, 14-15, 15-16, 16-17, 17-18, 18-19 or 19-20 consecutive amino acid residues of a MuV SH protein. In one embodiment, the MuV SH protein has the sequence as set forth in SEQ ID NO: 115.

In one embodiment, P comprises at least the sequence PAIQPPLY (SEQ ID NO: 98). In one embodiment P consists of a sequence selected from the group consisting of:

(SEQ ID NO: 98)
PAIQPPLY,
(SEQ ID NO: 97)
PAIQPPLYL,
(SEQ ID NO: 96)
PAIQPPLYLT,
(SEQ ID NO: 95)
PAIQPPLYLTF,
(SEQ ID NO: 94)
PAIQPPLYLTFL,
(SEQ ID NO: 93)
PAIQPPLYLTFLL,
(SEQ ID NO: 92)
PAIQPPLYLTFLLL,
(SEQ ID NO: 116)
PAIQPPLYLTFLLLI,
(SEQ ID NO: 117)
PAIQPPLYLTFLLLIL,
(SEQ ID NO: 118)
PAIQPPLYLTFLLLILL,
(SEQ ID NO: 119)
PAIQPPLYLTFLLLILLY,
(SEQ ID NO: 120)
PAIQPPLYLTFLLLILLYL,
and
(SEQ ID NO: 121)
PAIQPPLYLTFLLLILLYLI,

or a variant thereof.

In one embodiment P consists of a sequence selected from the group consisting of SEQ ID NO: 92-98 and 116-121 or a variant of any one of SEQ ID NO: 92-98 and 116-121 having one or more amino acid substitutions, such as one or more conservative amino acid substitutions.

In one embodiment P consists of a sequence selected from the group consisting of SEQ ID NO: 92-98 and 116-121 or a variant of any one of SEQ ID NO: 92-98 and 116-121 having one amino acid substitution, such as a variant of any one of SEQ ID NO: 92-98 and 116-121 having two amino acid substitutions, such as a variant of any one of SEQ ID NO: 92-98 and 116-121 having three amino acid substitutions, such as a variant of any one SEQ ID NO: 92-98 and 116-121 having four amino acid substitutions, such as a variant of any one of SEQ ID NO: 92-98 and 116-121 having five amino acid substitutions.

In one embodiment P is acetylated. In one embodiment P comprises a N-terminal amino acid residue which is acetylated (COCH3 or Ac—). In one embodiment P comprises a C-terminal amino acid residue which is amidated (—NH2).

In one embodiment, P comprises at least the sequence PAIQPPLY (SEQ ID NO: 98). In one embodiment P consists of a sequence selected from the group consisting of:

(SEQ ID NO: 91)
MPAIQPPLY,
(SEQ ID NO: 90)
MPAIQPPLYL,
(SEQ ID NO: 89)
MPAIQPPLYLT,
(SEQ ID NO: 88)
MPAIQPPLYLTF,
(SEQ ID NO: 87)
MPAIQPPLYLTFL,
(SEQ ID NO: 86)
MPAIQPPLYLTFLL,
(SEQ ID NO: 85)
MPAIQPPLYLTFLLL,
(SEQ ID NO: 122)
MPAIQPPLYLTFLLLI,
(SEQ ID NO: 123)
MPAIQPPLYLTFLLLIL,
(SEQ ID NO: 124)
MPAIQPPLYLTFLLLILL,
(SEQ ID NO: 125)
MPAIQPPLYLTFLLLILLY,
and
(SEQ ID NO: 126)
MPAIQPPLYLTFLLLILLYL,

or a variant thereof.

In one embodiment P consists of a sequence selected from the group consisting of SEQ ID NO: 85-91 and 122-126 or a variant of any one of SEQ ID NO: 85-91 and 122-126 having one or more amino acid substitutions, such as one or more conservative amino acid substitutions.

In one embodiment P consists of a sequence selected from the group consisting of SEQ ID NO: 85-91 and 122-126 or a variant of any one of SEQ ID NO: 85-91 and 122-126 having one amino acid substitution, such as a variant of any one of SEQ ID NO: 85-91 and 122-126 having two amino acid substitutions, such as a variant of any one of SEQ ID NO: 85-91 and 122-126 having three amino acid substitutions, such as a variant of any one SEQ ID NO: 85-91 and 122-126 having four amino acid substitutions, such as a variant of any one of SEQ ID NO: 85-91 and 122-126 having five amino acid substitutions.

In one embodiment, the present disclosure provides a compound of formula (I)

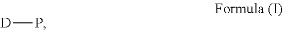

wherein
D comprises or consists of a drug; and
P is selected from the group consisting of:
PAIQPPLY (SEQ ID NO: 98),

(SEQ ID NO: 97)
PAIQPPLYL,
(SEQ ID NO: 96)
PAIQPPLYLT,
(SEQ ID NO: 95)
PAIQPPLYLTF,
(SEQ ID NO: 94)
PAIQPPLYLTFL,
(SEQ ID NO: 93)
PAIQPPLYLTFLL,
(SEQ ID NO: 92)
PAIQPPLYLTFLLL,
(SEQ ID NO: 116)
PAIQPPLYLTFLLLI,
(SEQ ID NO: 117)
PAIQPPLYLTFLLLIL,
(SEQ ID NO: 118)
PAIQPPLYLTFLLLILL,
(SEQ ID NO: 119)
PAIQPPLYLTFLLLILLY,
(SEQ ID NO: 120)
PAIQPPLYLTFLLLILLYL,
(SEQ ID NO: 121)
PAIQPPLYLTFLLLILLYLI,
(SEQ ID NO: 91)
MPAIQPPLY,
(SEQ ID NO: 90)
MPAIQPPLYL,
(SEQ ID NO: 89)
MPAIQPPLYLT,
(SEQ ID NO: 88)
MPAIQPPLYLTF,
(SEQ ID NO: 87)
MPAIQPPLYLTFL,
(SEQ ID NO: 86)
MPAIQPPLYLTFLL,
(SEQ ID NO: 85)
MPAIQPPLYLTFLLL,
(SEQ ID NO: 122)
MPAIQPPLYLTFLLLI,
(SEQ ID NO: 123)
MPAIQPPLYLTFLLLIL,
(SEQ ID NO: 124)
MPAIQPPLYLTFLLLILL,
(SEQ ID NO: 125)
MPAIQPPLYLTFLLLILLY,
and
(SEQ ID NO: 126)
MPAIQPPLYLTFLLLILLYL,

or a variant thereof comprising one or more amino acid substitutions, wherein D is conjugated to P, optionally via a linker L.

In one embodiment P is acetylated. In one embodiment P comprises a N-terminal amino acid residue which is acetylated (COCH3 or Ac—). In one embodiment P comprises a C-terminal amino acid residue which is amidated (—NH2).

In one embodiment, the variant of P is a functional variant of P.

In one embodiment, the variant of P or the functional variant of P is a peptide selected from the group consisting of any one of SEQ ID NO: 85-98 or 116-126 having one or more amino acid substitutions, such as having 1, 2, 3, 4 or 5 amino acid substitutions. In one embodiment, the variant of P or the functional variant of P is a peptide selected from the group consisting of any one of SEQ ID NO: 85-98 or 116-126 having one amino acid substitution. In one embodiment, the variant of P or the functional variant of P is a peptide selected from the group consisting of any one of SEQ ID NO: 85-98 or 116-126 having two amino acid substitutions. In one embodiment, the variant of P or the functional variant of P is a peptide selected from the group consisting of any one of SEQ ID NO: 85-98 or 116-126 having three amino acid substitutions. In one embodiment, the variant of P or the functional variant of P is a peptide selected from the group consisting of any one of SEQ ID NO: 85-98 or 116-126 having four amino acid substitutions. In one embodiment, the variant of P or the functional variant of P is a peptide selected from the group consisting of any one of SEQ ID NO: 85-98 or 116-126 having five amino acid substitutions.

In one embodiment, the one or more amino acid substitutions are conservative amino acid substitutions.

In one embodiment, the variant of the fragment P or the functional variant of the fragment P is acetylated. In one embodiment, the variant of the fragment P or the functional variant of the fragment P comprises a N-terminal amino acid residue which is acetylated (COCH3 or Ac—). In one embodiment, the variant of the fragment P or the functional variant of the fragment P comprises C-terminal amino acid residue which is amidated (—NH2).

The genetic code specifies 20 standard amino acids naturally incorporated into polypeptides (proteinogenic): Ala, Arg, Asn, Asp, Cys, Gln, Glu, Gly, His, IIe, Leu, Lys, Met, Phe, Pro, Ser, Tyr, Thr, Trp, Val, and 2 which are incorporated into proteins by unique synthetic mechanisms: Sec (selenocysteine, or U) and Pyl (pyrrolysine, O). These are all L-stereoisomers. The one or more amino acid substitutions according to the present disclosure may be a substitution to (or from) any one of these amino acids.

Aside from the 22 standard or natural amino acids, there are many other non-naturally occurring amino acids (non-proteinogenic or non-standard). They are either not found in proteins, or are not produced directly and in isolation by standard cellular machinery. Non-standard amino acids are usually formed through modifications to standard amino acids, such as post-translational modifications. Examples of unnatural amino acid residues are Abu, Aib, Nle (Norleucine), DOrn (D-ornithine, deguanylated arginine), Nal (beta-2-naphthyl-alanine), D-Nal (beta-2-naphthyl-D-alanine), DArg, DTrp, DPhe and DVal. The one or more amino acid substitutions according to the present disclosure may be a substitution to any one of these amino acids.

Any amino acids according to the present disclosure may be in the L- or D-configuration. If nothing is specified, reference to the L-isomeric form is preferably meant.

The term peptide and polypeptide also embraces post-translational modifications introduced by chemical or enzyme-catalyzed reactions, as are known in the art. Such post-translational modifications can be introduced prior to partitioning, if desired. Also, functional equivalents may comprise chemical modifications such as ubiquitination, labeling (e.g., with radionuclides, various enzymes, etc.), pegylation (derivatization with polyethylene glycol), or by insertion (or substitution by chemical synthesis) of amino acids, which do not normally occur in human proteins (e.g. ornithine).

### Drug “D”

The present disclosure provides means for targeted delivery of a drug (D) to and/or across a membrane or barrier harbouring GPR125. The drug may be any drug of interest to be delivered to and/or across a membrane or barrier harbouring GPR125. In one embodiment, D is a pharmaceutical drug, a medicinal product or an active pharmaceutical ingredient.

In one embodiment, D comprises or consists of a drug that targets a component in the central nervous system (CNS), the cerebrospinal fluid, the brain, the kidney, the testes, the cornea, and/or the lung.

In one embodiment, D comprises or consists of a drug that targets a component in the central nervous system (CNS), the cerebrospinal fluid, and/or the brain.

In one embodiment, D comprises or consists of a drug that targets a component in the testes.

In one embodiment, D is a small molecule drug, a peptide drug, or a protein drug. In one embodiment, D is a small molecule drug. In one embodiment, D is a peptide drug. In one embodiment, D is a protein drug. In one embodiment, D is a biologic. In one embodiment, D is an antibody-based drug, such as an antibody.

In one embodiment, D is selected from the group consisting of antidepressants, anticancer agents, painkillers, antidiabetic drugs, neurodegenerative drugs, anti-hypertensive drugs, diuretic drugs, and anti-inflammatory drugs.

The glucagon-like peptide-1 (GLP-1) is a multifaceted hormone with broad pharmacological potential. Among the numerous metabolic effects of GLP-1 are the glucose-dependent stimulation of insulin secretion, decrease of gastric emptying, inhibition of food intake, increase of natriuresis and diuresis, and modulation of rodent B-cell proliferation. GLP-1 also has cardio- and neuroprotective effects, decreases inflammation and apoptosis, and has implications for learning and memory, reward behavior, and palatability. Biochemically modified for enhanced potency and sustained action, GLP-1 receptor agonists are successfully in clinical use for the treatment of type-2 diabetes, and several GLP-1-based pharmacotherapies are in clinical evaluation for the treatment of obesity.

Glucagon-like peptide-1 (GLP-1) is a 30- or 31-amino-acid-long peptide hormone deriving from the tissue-specific posttranslational processing of the proglucagon peptide. The initial product GLP-1 (1-37) is susceptible to amidation and proteolytic cleavage, which gives rise to the two truncated and equipotent biologically active forms, GLP-1 (7-36) amide and GLP-1 (7-37). Active GLP-1 protein secondary structure includes two α-helices from amino acid position 13-20 and 24-35 separated by a linker region.

In one embodiment D is a peptide based GLP-1 receptor agonist. In one embodiment D is a peptide based GLP-1 receptor agonist. In one embodiment D is a GLP-1 derived peptide. In one embodiment D is a GLP-1 analogue. In one embodiment D is an Exendin-4 derived peptide. In one embodiment D is an Exendin-4 analogue.

In one embodiment, D is a GLP-1 receptor agonist, such as a GLP-1 receptor agonist selected from the group consisting of exenatide, liraglutide, lixisenatide, albiglutide, dulaglutide, and semaglutide.

Semaglutide is a modified analogue of human GLP-1(7-37) peptide. Compared to the amino acid sequence of GLP-1(7-37) peptide, the semaglutide peptide sequence contains two amino acid substitutions (Ala8 to Aib8 (2-aminoisobutyric acid), Lys34 to Arg34) and a modification at lysine 26 side chain with fatty diacid moiety.

In one embodiment, D is GLP-1 or a variant, a fragment, or a variant of a fragment thereof. In one embodiment, D is native GLP-1 (1-37) or a variant, a fragment, or a variant of a fragment thereof. In one embodiment, D is GLP-1(7-37) or a variant, a fragment, or a variant of a fragment thereof. In one embodiment, D is GLP-1(7-36) such as GLP-1(7-36) amide or a variant, a fragment, or a variant of a fragment thereof. In one embodiment, D is GLP-1(6-37) or a variant, a fragment, or a variant of a fragment thereof. In one embodiment, D is GLP-1(6-36) such as GLP-1(6-36) amide or a variant, a fragment, or a variant of a fragment thereof. In one embodiment said GLP-1 peptide is C-terminally amidated.

In one embodiment, D is GLP-1(7-36) or a variant, a fragment, or a variant of a fragment thereof. In one embodiment, D is GLP-1(7-36) comprising the sequence as set forth in SEQ ID NO: 127. In one embodiment, D is GLP-1(7-36) consisting of the sequence as set forth in SEQ ID NO: 127. In one embodiment, D is GLP-1(7-36) comprising a linker or a spacer on Lys26. In one embodiment, D is GLP-1(7-36) and is conjugated to the MuV SH protein or a variant, a fragment, or a variant of a fragment thereof via a linker (L). In one embodiment, D is GLP-1(7-36) and is conjugated to the MuV SH protein or a variant, a fragment, or a variant of a fragment thereof directly (without a linker).

In one embodiment, D is GLP-1(7-37) or a variant, a fragment, or a variant of a fragment thereof. In one embodiment, D is GLP-1(7-37) comprising the sequence as set forth in SEQ ID NO: 132. In one embodiment, D is GLP-1(7-37) consisting of the sequence as set forth in SEQ ID NO: 132.

In one embodiment, D is a variant of GLP-1, such as a variant of GLP-1(7-37), such as a variant of GLP-1(7-36). In one embodiment, the variant of GLP-1, GLP-1 (7-37) or GLP-1 (7-36) comprises one or more amino acid substitutions, one or more amino acid deletions, and/or one or more amino acid insertions. In one embodiment, the one or more amino acid substitution(s) is a conservative amino acid substitution, such as an amino acid substitution not affecting the structure or function of the GLP-1peptide.

In one embodiment, the variant of GLP-1, GLP-1 (7-37) or GLP-1 (7-36) comprises less than 10 amino acid substitutions, deletions and/or insertions, such as less than 9 amino acid substitutions, deletions and/or insertions, such as less than 8 amino acid substitutions, deletions and/or insertions, such as less than 7 amino acid substitutions, deletions and/or insertions, such as less than 6 amino acid substitutions, deletions and/or insertions, such as less than 5 amino acid substitutions, deletions and/or insertions, such as less than 4 amino acid substitutions, deletions and/or insertions, such as less than 3 amino acid substitutions, deletions and/or insertions, such as less than 2 amino acid substitutions, deletions and/or insertions, such as less than 1 amino acid substitutions, deletions and/or insertions.

In one embodiment, the variant of GLP-1, GLP-1 (7-37) or GLP-1 (7-36) comprises one or more amino acid substitutions as compared to the native sequence, such as compared to GLP-1, SEQ ID NO: 127 (GLP-1 7-36) or SEQ ID NO: 132 (GLP-1 7-37). In one embodiment, the variant of GLP-1, GLP-1 (7-37) or GLP-1 (7-36) comprises one amino acid substitution, such as two amino acid substitutions, such as three amino acid substitutions, such as 4 amino acid substitutions, such as 5 amino acid substitutions, such as 6 amino acid substitutions, such as 7 amino acid substitutions, such as 8 amino acid substitutions.

In one embodiment, the variant of GLP-1 comprises one or more amino acid substitutions as compared to Semaglutide.

In one embodiment, the variant of GLP-1, GLP-1 (7-37) or GLP-1 (7-36) comprises one or more fatty acid moiety/moieties, such as fatty diacid moieties.

In one embodiment, D is a variant of GLP-1, such as a variant of GLP-1(7-37), such as a variant of GLP-1(7-36). In one embodiment, the variant comprises an amino acid substitution wherein a Lys (lysine residue) is introduced at any position in the GLP-1 sequence. In one embodiment, D is a variant of GLP-1 containing a Lys at any position selected from position 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and 37 of SEQ ID NO: 127 and SEQ ID NO: 132.

In one embodiment, D is GLP-1 or a variant of GLP-1, such as GLP-1(7-37) or a variant thereof, such as GLP-1(7-36) or a variant thereof, and is conjugated to a MuV SH protein or a variant, a fragment, or a variant of a fragment thereof according to the present disclosure directly.

In one embodiment, D is GLP-1 or a variant of GLP-1, such as GLP-1(7-37) or a variant thereof, such as GLP-1(7-36) or a variant thereof, and is conjugated to a MuV SH protein or a variant, a fragment, or a variant of a fragment thereof according to the present disclosure via a linker (L). In one embodiment, the linker is a spacer. In one embodiment, D is GLP-1 or a variant of GLP-1 according to the present disclosure comprising a linker or a spacer on Lys6, Lys7, Lys8, Lys9, Lys10, Lys11, Lys12, Lys13, Lys14, Lys15, Lys16, Lys17, Lys18, Lys19, Lys20, Lys21, Lys22, Lys23, Lys24, Lys25, Lys26, Lys27, Lys28, Lys29, Lys30, Lys31, Lys32, Lys33, Lys34, Lys35, Lys36 or Lys37. In one embodiment said linker (L) or spacer on said lysine residue of the GLP-1 protein (D) conjugates the GLP-1 protein (D) to the MuV SH protein (P).

In one embodiment, D is GLP-1 or a variant thereof according to the present disclosure containing a naturally occurring Lys. In one embodiment, D is GLP-1 or a variant thereof according to the present disclosure containing an artificially introduced Lys. In one embodiment, the naturally occurring or artificially introduced Lys may be found at position 26 or 34, as compared to the sequence of native GLP-1. In one embodiment, the naturally occurring or artificially introduced Lys may be found at position 20 or 28, as compared to the sequence of GLP-1 7-36 SEQ ID NO: 127). Reference to ‘Lys26’ is meant to refer to the lysine residue found in GLP-1 at position 26; corresponding to amino acid no. 20 in GLP-1 (7-36) and GLP-1 (7-37).

In one embodiment, D is GLP-1 or a variant of GLP-1 according to the present disclosure comprising a linker or a spacer on Lys26 or Lys34. In one embodiment, D is GLP-1 or a variant of GLP-1 according to the present disclosure conjugated to a MuV SH protein or a variant, a fragment, or a variant of a fragment thereof according to the present disclosure via a linker or a spacer on Lys26 or Lys34. In one embodiment said Linker is a PEG-linker, such as a PEG2-linker.

Exendin-4 is a 39 amino acid peptide agonist of the glucagon-like peptide (GLP) receptor that promotes insulin secretion.

In one embodiment, D is Exendin-4 or a variant, a fragment, or a variant of a fragment thereof. In one embodiment, D is native Exendin-4 (SEQ ID NO: 130) or a variant, a fragment, or a variant of a fragment thereof. Native Exendin-4 is also known as Exendin-4(1-39) and these terms will be used interchangeably to refer to the same peptide. In one embodiment Exendin-4 is C-terminally amidated (Exendin-4 amide).

In one embodiment, D is Exendin-4(1-39) or a variant, a fragment, or a variant of a fragment thereof. In one embodiment, D is Exendin-4(1-39) comprising the sequence as set forth in SEQ ID NO: 130. In one embodiment, D is Exendin-4(1-39) consisting of the sequence as set forth in SEQ ID NO: 130. In one embodiment, D is a fragment of Exendin-4(1-39). In one embodiment, D is Exendin-4 comprising a linker or a spacer on Lys27. In one embodiment, D is Exendin-4 and is conjugated to the MuV SH protein or a variant, a fragment, or a variant of a fragment thereof according to the present disclosure via a linker (L). In one embodiment, the linker is a spacer. In one embodiment, D is Exendin-4 or a variant thereof and is conjugated to the MuV SH protein or a variant, a fragment, or a variant of a fragment thereof according to the present disclosure directly (without a linker).

In one embodiment, D is a variant of Exendin-4, such as a variant of Exendin-4(1-39) or a fragment thereof. In one embodiment, the variant of Exendin-4 comprises one or more amino acid substitutions, one or more amino acid deletions, and/or one or more amino acid insertions. In one embodiment, the one or more amino acid substitution(s) is a conservative amino acid substitution, such as an amino acid substitution not affecting the structure or function of the Exendin-4.

In one embodiment, the variant of Exendin-4 comprises less than 10 amino acid substitutions, deletions and/or insertions, such as less than 9 amino acid substitutions, deletions and/or insertions, such as less than 8 amino acid substitutions, deletions and/or insertions, such as less than 7 amino acid substitutions, deletions and/or insertions, such as less than 6 amino acid substitutions, deletions and/or insertions, such as less than 5 amino acid substitutions, deletions and/or insertions, such as less than 4 amino acid substitutions, deletions and/or insertions, such as less than 3 amino acid substitutions, deletions and/or insertions, such as less than 2 amino acid substitutions, deletions and/or insertions, such as less than 1 amino acid substitutions, deletions and/or insertions.

In one embodiment, the variant of Exendin-4 comprises one or more amino acid substitutions as compared to the native sequence, such as compared to SEQ ID NO: 130. In one embodiment, the variant of Exendin-4 comprises one amino acid substitution, such as two amino acid substitutions, such as three amino acid substitutions, such as 4 amino acid substitutions, such as 5 amino acid substitutions, such as 6 amino acid substitutions, such as 7 amino acid substitutions, such as 8 amino acid substitutions compared to Exendin-4 (SEQ ID NO: 130).

In one embodiment, D is a variant of Exendin-4. In one embodiment, the variant comprises an amino acid substitution wherein a Lys (lysine residue) is introduced at any position in the Exendin-4 sequence. In one embodiment, D is a variant of Exendin-4 containing a Lys at any position selected from position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 and 39 of Exendin-4 (SEQ ID NO: 130).

In one embodiment, D is Exendin-4 and is conjugated to the MuV SH protein or a variant, a fragment, or a variant of a fragment thereof (P) via a linker (L). In one embodiment, the linker is a spacer. In one embodiment, D is a variant of Exendin-4 comprising a linker or a spacer on Lys1, Lys2, Lys3, Lys4, Lys5, Lys6, Lys7, Lys8, Lys9, Lys10, Lys11, Lys12, Lys13, Lys14, Lys15, Lys16, Lys17, Lys18, Lys19, Lys20, Lys21, Lys22, Lys23, Lys24, Lys25, Lys26, Lys27, Lys28, Lys29, Lys30, Lys31, Lys32, Lys33, Lys34, Lys35, Lys36, Lys37, Lys38 or Lys39.

In one embodiment, D is Exendin-4 or a variant thereof according to the present disclosure containing a naturally occurring Lys. In one embodiment, D is Exendin-4 or a variant thereof according to the present disclosure containing an artificially introduced Lys. In one embodiment, the naturally occurring or artificially introduced Lys may be found at position 12 or 27, as compared to the sequence of Exendin(1-39) (SEQ ID NO: 130).

In one embodiment, D is Exendin-4 or a variant thereof according to the present disclosure and is conjugated to a MuV SH protein or a variant, a fragment, or a variant of a fragment thereof according to the present disclosure via a linker (L). In one embodiment, the linker is a spacer. In one embodiment, D is Exendin-4 or a variant thereof according to the present disclosure and is conjugated to a MuV SH protein or a variant, a fragment, or a variant of a fragment thereof according to the present disclosure via a linker (L) on Lys12 or Lys27 of said Exendin-4. In one embodiment said Linker is a PEG-linker, such as a PEG2 linker.

### Linker

In one embodiment the MuV SH protein is hydrophobic. In one embodiment the solubility of the peptide drug conjugate of the present disclosure comprising a MuV SH protein (P) may be determined by the solubility of the drug D. In one embodiment the addition of a linker or a spacer between the drug D and the peptide P may aid in improving the solubility of the peptide drug conjugate of the present invention.

The MuV SH protein or a variant, a fragment, or a variant of a fragment thereof (peptide P) is in one embodiment conjugated to the drug D via a linker (L). The linker may be a cleavable linker or a non-cleavable linker.

A cleavable linker will allow for release of the drug from the MuV SH protein or fragment thereof following delivery to and/or across a membrane or barrier harbouring GPR125. Thus, in one embodiment, L is a cleavable linker. Examples of cleavable linkers include but are not limited to acid cleavable linkers, pH cleavable linker, and enzyme cleavable linker.

A non-cleavable linker may be desirable where targeted delivery of the drug to a membrane or barrier harbouring GPR125 is desired, thereby allowing for controlling the position of the drug at the site of the membrane to perform its action. Thus, in one embodiment, L is a non-cleavable linker. Examples of non-cleavable linkers include but are not limited to polyethylene glycol linker (PEG linker), and Glycine serine linker (GS linker.

In one embodiment, the linker is referred to as a spacer. In one embodiment, the linker is a PEG linker. also referred to as a PEG spacer. In one embodiment the linker is a PEG2 linker, also referred to as a PEG2 spacer.

In one embodiment, the linker, L, is selected from the group consisting of PEG2-20, aliphatic spacers with a length from 4-60 atoms, linear and branched amine spacers such as HMDA, linear and branched amide spacers, and peptide spacers such as Glutathione (GSH).

In one embodiment, the linker (L) is attached to a naturally occurring Lys in the drug “D”. In one embodiment, the linker (L) is attached to a Lys which has been artificially introduced in the drug “D”.

In one embodiment, the linker (L) is attached to a Lys in the drug “D”, wherein D is a GLP-1 or exendin-4 based drug according to the present disclosure.

In one embodiment, the compound of the present disclosure does not comprise a linker.

### GPR125 and Membrane or Barrier Harbouring GPR125

In one embodiment, the membrane or barrier harbouring GPR125 is selected from the group consisting of choroid plexus, kidney epithelium, urogenital epithelium, blood-ocular barriers, and lung epithelium.

In one embodiment, the membrane or barrier harbouring GPR125 is the blood-cerebrospinal fluid barrier (BCSFB), the blood-aqueous-barrier (BAB) or the blood-testes-barrier (BTB).

In a preferred embodiment, the membrane or barrier harbouring GPR125 is the blood-cerebrospinal fluid barrier (BCSFB).

GPR125 is also known to be expressed in high level in tumours. Thus in one embodiment, the membrane or barrier harbouring GPR125 is present in a tumour.

### Mechanism of Action

In one embodiment, the compound of the present disclosure is capable of interacting with GPR125. In one embodiment, P is capable of interacting with GPR125.

In one embodiment, the compound of the present disclosure provides targeted delivery of D across a membrane or barrier harbouring GPR125 and/or to a site expressing GPR125. In one embodiment, the compound of the present disclosure is for use in a method of targeted delivery of D across a membrane or barrier harbouring GPR125 and/or to a site expressing GPR125. In one embodiment, the compound of the present disclosure is for use in a method of targeted delivery of D across a membrane or barrier harbouring GPR125 and/or to a site expressing GPR125, wherein the membrane or barrier harbouring GPR125 is selected from the group consisting of choroid plexus, kidney epithelium, urogenital epithelium, blood-ocular barriers, and lung epithelium.

In one embodiment, interaction of the compound, or of P of the compound, with GPR125 facilitates crossing of the compound across a membrane or barrier harbouring the GPR125. In one embodiment, interaction of the compound, or of P of the compound, with GPR125 facilitates crossing of D across a membrane or barrier harbouring the GPR125.

In one embodiment, interaction of the compound, or of P of the compound, with GPR125 facilitates internalization of the compound across a membrane or barrier harbouring the GPR125. In one embodiment, interaction of the compound, or of P of the compound, with GPR125 facilitates internalization of D across a membrane or barrier harbouring the GPR125.

In one embodiment, interaction of the compound, or of P of the compound, with GPR125 results in reduced membrane integrity of the membrane harbouring the GPR125. In one embodiment, the reduced membrane integrity results in uptake of the compound across the membrane. In one embodiment, the reduced membrane integrity results in uptake of D across the membrane.

In one embodiment, the compound of the present disclosure is capable of crossing of the blood brain barrier, such as the blood-cerebrospinal fluid barrier, for example the choroid plexus.

In one embodiment, the compound of the present disclosure provides targeted delivery of the compound or D to the cerebrospinal fluid. In one embodiment, the compound of the present disclosure is for use in a method for targeted delivery of the compound or D to the cerebrospinal fluid.

In one embodiment, interaction of the compound, or of P of the compound, with GPR125 facilitates targeted delivery of the compound or D to a tumour, such as to a tumour located in the CNS, for example to a brain tumour, such as to a brain tumour selected from the group consisting of astrocytic tumour, oligodendroglial tumour, glioblastoma, pituitary tumour, pineal gland tumour, ependymomas, craniopharygiomas, primary central nervous system lymphomas, meningiomas, and schwannomas.

In one embodiment, the compound of the present disclosure is provided for use in targeted delivery of D to and/or across a membrane or barrier harbouring GPR125.

In one embodiment, the compound of the present disclosure is provided for use in targeted delivery of D to the CNS, such as to the brain.

In one embodiment, the compound of the present disclosure is provided for use in targeted delivery of D to the cerebrospinal fluid.

In one embodiment, the compound of the present disclosure is provided for use in targeted delivery of D to the eyeball.

In one embodiment, a method of preparing a drug which is permeable to a membrane or barrier harbouring GPR125 is provided, such as permeable to the blood brain barrier, such as permeable to the blood-cerebrospinal fluid barrier, such as the choroid plexus, the method comprising covalently conjugating the drug to a mumps virus short hydrophobic protein (MuV SH protein), or a variant, a fragment or a variant of a fragment thereof.

In one embodiment, a method for delivering a drug across a membrane or barrier harbouring GPR125 is provided, such as delivering a drug across the blood-cerebrospinal fluid barrier, the method comprising providing a compound as defined herein above. In one embodiment, the method of delivery is prepared in vitro or ex vivo.

### Indications and Method of Treatment

The compounds of the present disclosure provide means for treatment of diseases which are treated by targeting of a physiological component which is accessible only through crossing of a membrane or barrier harbouring GPR125, such as through crossing of choroid plexus, kidney epithelium, urogenital epithelium, blood-ocular barriers, and lung epithelium.

Thus, in one embodiment the compound of the present disclosure is provided for use as a medicament.

In one embodiment, the compound of the present disclosure is provided for use in the treatment of a CNS disease or disorder, a kidney disease or disorder, a urogenital disease or disorder, an ocular disease or disorder, or a lung disease or disorder.

In one embodiment, a method for treatment of a CNS disease or disorder, a kidney disease or disorder, a urogenital disease or disorder, an ocular disease or disorder, or a lung disease or disorder is provided, said method comprising administering a therapeutically effective amount of the compound as disclosed herein to a subject in need thereof.

In one embodiment, use of a compound as disclosed herein for the manufacture of a medicament for treatment of a CNS disease or disorder, a kidney disease or disorder, a urogenital disease or disorder, an ocular disease or disorder, a metabolic disorder, brain/CNS metastasis, or a lung disease or disorder is provided.

In one embodiment, the CNS disease or disorder is selected from the group consisting of neurodegenerative diseases, mental disorders, infectious disorder, headache or migraine, brain tumours, and brain/CNS metastasis.

In one embodiment, the neurodegenerative disorder is selected from the group consisting of Alzheimer's disease, Parkinson's disease, amyotrophic lateral sclerosis (ALS), Huntington's disease, and multiple sclerosis.

In one embodiment, the mental disorder is selected from the group consisting of dementia, depression, schizophrenia, bipolar disorder, anxiety disorder, eating disorder, and addiction.

In one embodiment, the infectious disorder is selected from the group consisting of meningitis and encephalitis.

I one embodiment, the metabolic disorder is a centrally regulated metabolic disorder. In one embodiment, the compound of the present disclosure provides regulation of appetite. In one embodiment, the metabolic disorder is selected from the group consisting of metabolic disease, type 2 diabetes, and obesity.

In one embodiment, the compound of the present disclosure is provided for use in the treatment of obesity. In one embodiment, the compound of the present disclosure is provided for use in a method of appetite regulation such as appetite reduction. In one embodiment, the compound of the present disclosure is provided for use in a method of reducing food intake

In one embodiment, the CNS disease or disorder is headache or migraine.

In one embodiment, the kidney disease or disorder is selected from the group consisting of Chronic kidney disease (CKD) and acute glomerulonephritis. In one embodiment, the ocular disease or disorder is melanomas in the inner eye.

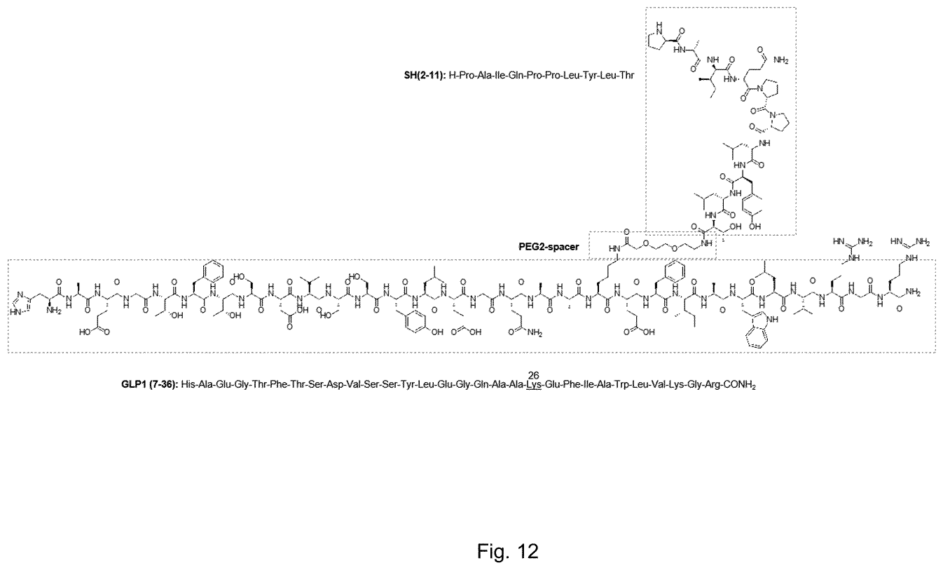

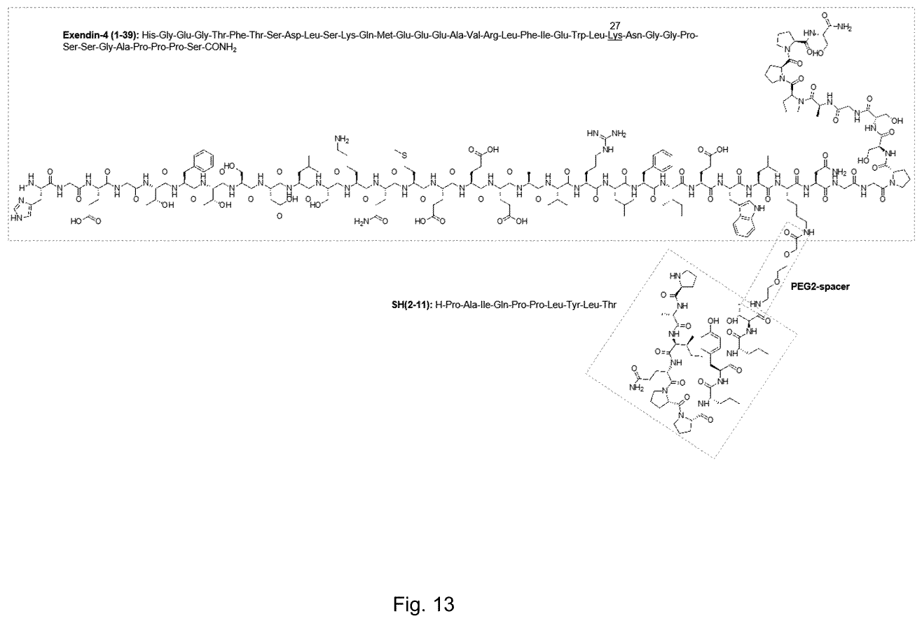

In one embodiment, the brain tumour is selected from the group consisting of astrocytic tumour, oligodendroglial tumour, glioblastoma, pituitary tumour, pineal gland tumour, ependymomas, craniopharygiomas, primary central nervous system lymphomas, meningiomas, and schwannomas.

| SEQ | | |
|---|---|---|
| ID NO: | Sequence | Comment |
| 1 | PAIX₁PPX₂X₃X₄T FLLLX₅LLX₆LIX₇TLYVW | Genotype G and vaccine strains |
| | X₈X₉X₁₀X₁₁X₁₂X₁₃X₁₄X₁₅TX₁₆VRX₁₇AX₁₈ | 2-57 |
| | LX₁₉QRSX₂₀X₂₁X₂₂WX₂₃X₂₄DX₂₅X₂₆L | Consensus sequence |
| | wherein | |
| | X₁ is Q or R | |
| | X₂ is L or S | |
| | X₃ is Y or H | |
| | X₄ is L or P | |
| | X₅ is I or T | |
| | X₆ is Y or S | |
| | X₇ is I, T or V | |
| | X₈ is I or T | |
| | X₉ is I or T | |
| | X₁₀ is L or S | |
| | X₁₁ is T or A | |
| | X₁₂ is V or I | |
| | X₁₃ is T or N | |
| | X₁₄ is Y or H | |
| | X₁₅ is K or N | |
| | X₁₆ is A or V | |
| | X₁₇ is H, P, Y or R | |
| | X₁₈ is A, T, or S | |
| | X₁₉ is Y or H | |
| | X₂₀ is F, C or S | |
| | X₂₁ is F, V or S | |
| | X₂₂ is H or R | |
| | X₂₃ is S, R or G | |
| | X₂₄ is F or L | |
| | X₂₅ is H or Q | |
| | X₂₆ is S, P, or T | |
| 2 | PAIX₁PPX₂YL TFLLLILLYL IX₃TLYVWIIL | Genotype G |
| | X₄VTYKTAVRX₅ AALYQRSX₆X₇H | 2-57 |
| | WX₈FDHX₉L | Consensus sequence |
| | wherein | |
| | X₁ is Q or R | |
| | X₂ is L or S | |
| | X₃ is I or T | |
| | X₄ is T or A | |
| | X₅ is H, P or R | |
| | X₆ is F or S | |
| | X₇ is F or V | |
| | X₈ is S or R | |
| | X₉ is S or T | |
| 3 | PAIQPPLX₁X₂ TFLLLX₃LLX₄L | Vaccine strains |
| | IX₅TLYVWX₆X₇X₈ TIX₉X₁₀X₁₁TX₁₂VRX₁₃ | 2-57 |
| | AX₁₄LX₁₅QRSX₁₆X₁₇R WX₁₈X₁₉DX₂₀X₂₁L | Consensus sequence |
| | wherein | |
| | X₁ is Y or H | |
| | X₂ is L or P | |
| | X₃ is I or T | |
| | X₄ is Y or S | |
| | X₅ is I or V | |
| | X₆ is I or T | |
| | X₇ is I or T | |
| | X₈ is L or S | |
| | X₉ is T or N | |
| | X₁₀ is Y or H | |
| | X₁₁ is K or N | |
| | X₁₂ is A or V | |
| | X₁₃ is Y or H | |
| | X₁₄ is A, T, or S | |
| | X₁₅ is H or Y | |
| | X₁₆ is F or C | |
| | X₁₇ is F or S | |
| | X₁₈ is S or G | |
| | X₁₉ is F or L | |
| | X₂₀ is H or Q | |
| | X₂₁ is S or P | |
| 4 | MPAIX₁PPX₂X₃X₄ TFLLLX₅LLX₆L | Genotype G and vaccine strains |
| | IX₇TLYVWX₈X₉X₁₀ | Full length |
| | X₁₁X₁₂X₁₃X₁₄X₁₅TX₁₆VRX₁₇ | Consensus sequence |
| | AX₁₈LX₁₉QRSX₂₀X₂₁X₂₂ | |
| | WX₂₃X₂₄DX₂₅X₂₆L | |
| | wherein | |
| | X₁ is Q or R | |
| | X₂ is L or S | |
| | X₃ is Y or H | |
| | X₄ is L or P | |
| | X₅ is I or T | |
| | X₆ is Y or S | |
| | X₇ is I, T or V | |
| | X₈ is I or T | |
| | X₉ is I or T | |
| | X₁₀ is L or S | |
| | X₁₁ is T or A | |
| | X₁₂ is V or I | |
| | X₁₃ is T or N | |
| | X₁₄ is Y or H | |
| | X₁₅ is K or N | |
| | X₁₆ is A or V | |
| | X₁₇ is H, P, Y or R | |
| | X₁₈ is A, T, or S | |
| | X₁₉ is Y or H | |
| | X₂₀ is F, C or S | |
| | X₂₁ is F, V or S | |
| | X₂₂ is H or R | |
| | X₂₃ is S, R or G | |
| | X₂₄ is F or L | |
| | X₂₅ is H or Q | |
| | X₂₆ is S, P, or T | |
| 5 | MPAIX₁PPX₂YL TFLLLILLYL IX₃TLYVWIIL | Genotype G |
| | X₄VTYKTAVRX₅ AALYQRSX₆X₇H | |
| | WX₈FDHX₉L | Full length |
| | wherein | Consensus sequence |
| | X₁ is Q or R | |
| | X₂ is L or S | |
| | X₃ is I or T | |
| | X₄ is T or A | |
| | X₅ is H, P or R | |
| | X₆ is F or S | |
| | X₇ is F or V | |
| | X₈ is S or R | |
| | X₉ is S or T | |
| 6 | MPAIQPPLX₁X₂ TFLLLX₃LLX₄L | Vaccine strains |
| | IX₅TLYVWX₆X₇X₈ TIX₉X₁₀X₁₁TX₁₂VRX₁₃ | Full length |
| | AX₁₄LX₁₅QRSX₁₆X₁₇R WX₁₈X₁₉DX₂₀X₂₁L | |
| | wherein | Consensus sequence |
| | X₁ is Y or H | |
| | X₂ is L or P | |
| | X₃ is I or T | |
| | X₄ is Y or S | |
| | X₅ is I or V | |
| | X₆ is I or T | |
| | X₇ is I or T | |
| | X₈ is L or S | |
| | X₉ is T or N | |
| | X₁₀ is Y or H | |
| | X₁₁ is K or N | |
| | X₁₂ is A or V | |
| | X₁₃ is Y or H | |
| | X₁₄ is A, T, or S | |
| | X₁₅ is H or Y | |
| | X₁₆ is F or C | |
| | X₁₇ is F or S | |
| | X₁₈ is S or G | |
| | X₁₉ is F or L | |
| | X₂₀ is H or Q | |
| | X₂₁ is S or P | |
| 7 | MPAIX₁PPX₂X₃X₄ TFLLL | Genotype G and vaccine strains |
| | wherein | Fragment (1-15) |
| | X₁ is Q or R | Consensus sequence |
| | X₂ is L or S | |
| | X₃ is Y or H | |
| | X₄ is L or P | |
| 8 | MPAIX₁PPX₂YL TFLLL | Genotype G |
| | wherein | Fragment (1-15) |
| | X₁ is Q or R | Consensus sequence |
| | X₂ is L or S | |
| 9 | MPAIQPPLX₁X₂ TFLLL | Vaccine strains |
| | wherein | Fragment (1-15) |
| | X₁ is Y or H | Consensus sequence |
| | X₂ is L or P | |
| 10 | MPAIX₁PPX₂X₃X₄ TFLL | Genotype G and vaccine strains |
| | wherein | Fragment (1-14) |
| | X₁ is Q or R | Consensus sequence |
| | X₂ is L or S | |
| | X₃ is Y or H | |
| | X₄ is L or P | |
| 11 | MPAIX₁PPX₂YL TFLL | Genotype G |
| | wherein | Fragment (1-14) |
| | X₁ is Q or R | Consensus sequence |
| | X₂ is L or S | |
| 12 | MPAIQPPLX₁X₂ TFLL | Vaccine strains |
| | wherein | Fragment (1-14) |
| | X₁ is Y or H | Consensus sequence |
| | X₂ is L or P | |
| 13 | MPAIX₁PPX₂X₃X₄ TFL | Genotype G and vaccine strains |
| | wherein | Fragment (1-13) |
| | X₁ is Q or R | Consensus sequence |
| | X₂ is L or S | |
| | X₃ is Y or H | |
| | X₄ is L or P | |
| 14 | MPAIX₁PPX₂YL TFL | Genotype G |
| | wherein | Fragment (1-13) |
| | X₁ is Q or R | Consensus sequence |
| | X₂ is L or S | |
| 15 | MPAIQPPLX₁X₂ TFL | Vaccine strains |
| | wherein | Fragment (1-13) |
| | X₁ is Y or H | Consensus sequence |
| | X₂ is L or P | |
| 16 | MPAIX₁PPX₂X₃X₄ TF | Genotype G and vaccine strains |
| | wherein | Fragment (1-12) |
| | X₁ is Q or R | Consensus sequence |
| | X₂ is L or S | |
| | X₃ is Y or H | |
| | X₄ is L or P | |
| 17 | MPAIX₁PPX₂YL TF | Genotype G |
| | wherein | Fragment (1-12) |
| | X₁ is Q or R | Consensus sequence |
| | X₂ is L or S | |
| 18 | MPAIQPPLX₁X₂ TF | Vaccine strains |
| | wherein | Fragment (1-12) |
| | X₁ is Y or H | Consensus sequence |
| | X₂ is L or P | |
| 19 | MPAIX₁PPX₂X₃X₄ T | Genotype G and vaccine strains |
| | wherein | Fragment (1-11) |
| | X₁ is Q or R | Consensus sequence |
| | X₂ is L or S | |
| | X₃ is Y or H | |
| | X₄ is L or P | |
| 20 | MPAIX₁PPX₂YL T | Genotype G |
| | wherein | Fragment (1-11) |
| | X₁ is Q or R | Consensus sequence |
| | X₂ is L or S | |
| 21 | MPAIQPPLX₁X₂ T | Vaccine strains |
| | wherein | Fragment (1-11) |
| | X₁ is Y or H | Consensus sequence |
| | X₂ is L or P | |
| 22 | MPAIX₁PPX₂X₃X₄ | Genotype G and vaccine strains |
| | wherein | Fragment (1-10) |
| | X₁ is Q or R | Consensus sequence |
| | X₂ is L or S | |
| | X₃ is Y or H | |
| | X₄ is L or P | |
| 23 | MPAIX₁PPX₂YL | Genotype G |
| | wherein | Fragment (1-10) |
| | X₁ is Q or R | Consensus sequence |
| | X₂ is L or S | |
| 24 | MPAIQPPLX₁X₂ | Vaccine strains |
| | wherein | Fragment (1-10) |
| | X₁ is Y or H | Consensus sequence |
| | X₂ is L or P | |
| 25 | MPAIX₁PPX₂X₃ | Genotype G and vaccine strains |
| | wherein | Fragment (1-9) |
| | X₁ is Q or R | Consensus sequence |
| | X₂ is L or S | |
| | X₃ is Y or H | |
| 26 | MPAIQPPLX₁ | Vaccine strains |
| | wherein | Fragment (1-9) |
| | X₁ is Y or H | Consensus sequence |
| 27 | MPAIX₁PPX₂ | Genotype G and vaccine strains |
| | wherein | Fragment (1-8) |
| | X₁ is Q or R | Consensus sequence |
| | X₂ is L or S | |
| 28 | MPAIQPPL | Vaccine strains and Genotype G |
| | | Genbank ID QFO38283.1 |
| | | Fragment (1-8) |
| 29 | MPAIX₁PP | Genotype G and vaccine strains |
| | wherein | Fragment (1-7) |
| | X₁ is Q or R | Consensus sequence |
| 30 | MPAIQPP | Vaccine strains and Genotype G |
| | | Genbank ID QFO38283.1 |
| | | Fragment (1-7) |
| 31 | MPAIX₁P | Genotype G and vaccine strains |
| | wherein | Fragment (1-6) |
| | X₁ is Q or R | Consensus sequence |
| 32 | MPAIQP | Vaccine strains and Genotype G |
| | | Genbank ID QFO38283.1 |
| | | Fragment (1-6) |
| 33 | MPAIX₁ | Genotype G and vaccine strains |
| | wherein | Fragment (1-5) |
| | X₁ is Q or R | Consensus sequence |
| 34 | MPAIQ | Vaccine strains and Genotype G |
| | | Genbank ID QFO38283.1 |
| | | Fragment (1-5) |
| 35 | PAIX₁PPX₂X₃X₄ TFLLL | Genotype G and vaccine strains |
| | wherein | Fragment (2-15) |
| | X₁ is Q or R | Consensus sequence |
| | X₂ is L or S | |
| | X₃ is Y or H | |
| | X₄ is L or P | |
| 36 | PAIX₁PPX₂YL TFLLL | Genotype G |
| | wherein | Fragment (2-15) |
| | X₁ is Q or R | Consensus sequence |
| | X₂ is L or S | |
| 37 | PAIQPPLX₁X₂ TFLLL | Vaccine strains |
| | wherein | Fragment (2-15) |
| | X₁ is Y or H | Consensus sequence |
| | X₂ is L or P | |
| 38 | PAIX₁PPX₂X₃X₄ TFLL | Genotype G and vaccine strains |
| | wherein | Fragment (2-14) |
| | X₁ is Q or R | Consensus sequence |
| | X₂ is L or S | |
| | X₃ is Y or H | |
| | X₄ is L or P | |
| 39 | PAIX₁PPX₂YL TFLL | Genotype G |
| | wherein | Fragment (2-14) |
| | X₁ is Q or R | Consensus sequence |
| | X₂ is L or S | |
| 40 | PAIQPPLX₁X₂ TFLL | Vaccine strains |
| | wherein | Fragment (2-14) |
| | X₁ is Y or H | Consensus sequence |
| | X₂ is L or P | |
| 41 | PAIX₁PPX₂X₃X₄ TFL | Genotype G and vaccine strains |
| | wherein | Fragment (2-13) |
| | X₁ is Q or R | Consensus sequence |
| | X₂ is L or S | |
| | X₃ is Y or H | |
| | X₄ is L or P | |
| 42 | PAIX₁PPX₂YL TFL | Genotype G |
| | wherein | Fragment (2-13) |
| | X₁ is Q or R | Consensus sequence |
| | X₂ is L or S | |
| 43 | PAIQPPLX₁X₂ TFL | Vaccine strains |
| | wherein | Fragment (2-13) |
| | X₁ is Y or H | Consensus sequence |
| | X₂ is L or P | |
| 44 | PAIX₁PPX₂X₃X₄ TF | Genotype G and vaccine strains |
| | wherein | Fragment (2-12) |
| | X₁ is Q or R | Consensus sequence |
| | X₂ is L or S | |
| | X₃ is Y or H | |
| | X₄ is L or P | |
| 45 | PAIX₁PPX₂YL TF | Genotype G |
| | wherein | Fragment (2-12) |
| | X₁ is Q or R | Consensus sequence |
| | X₂ is L or S | |
| 46 | PAIQPPLX₁X₂ TF | Vaccine strains |
| | wherein | Fragment (2-12) |
| | X₁ is Y or H | Consensus sequence |
| | X₂ is L or P | |
| 47 | PAIX₁PPX₂X₃X₄ T | Genotype G and vaccine strains |
| | wherein | Fragment (2-11) |
| | X₁ is Q or R | Consensus sequence |
| | X₂ is L or S | |
| | X₃ is Y or H | |
| | X₄ is L or P | |
| 48 | PAIX₁PPX₂YL T | Genotype G |
| | wherein | Fragment (2-11) |
| | X₁ is Q or R | Consensus sequence |
| | X₂ is L or S | |
| 49 | PAIQPPLX₁X₂ T | Vaccine strains |
| | wherein | Fragment (2-11) |
| | X₁ is Y or H | Consensus sequence |
| | X₂ is L or P | |
| 50 | PAIX₁PPX₂X₃X₄ | Genotype G and vaccine strains |
| | wherein | Fragment (2-10) |
| | X₁ is Q or R | Consensus sequence |
| | X₂ is L or S | |
| | X₃ is Y or H | |
| | X₄ is L or P | |
| 51 | PAIX₁PPX₂YL | Genotype G |
| | wherein | Fragment (2-10) |
| | X₁ is Q or R | Consensus sequence |
| | X₂ is L or S | |
| 52 | PAIQPPLX₁X₂ | Vaccine strains |
| | wherein | Fragment (2-10) |
| | X₁ is Y or H | Consensus sequence |
| | X₂ is L or P | |
| 53 | PAIX₁PPX₂X₃ | Genotype G and vaccine strains |
| | wherein | Fragment (2-9) |
| | X₁ is Q or R | Consensus sequence |
| | X₂ is L or S | |
| | X₃ is Y or H | |
| 54 | PAIQPPLX₁ | Vaccine strains |
| | wherein | Fragment (2-9) |
| | X₁ is Y or H | Consensus sequence |
| 55 | PAIX₁PPX₂ | Genotype G and vaccine strains |
| | wherein | Fragment (2-8) |
| | X₁ is Q or R | Consensus sequence |
| | X₂ is L or S | |
| 56 | PAIQPPL | Vaccine strains and Genotype G |
| | | Genbank ID QFO38283.1 |
| | | Fragment (2-8) |
| 57 | PAIX₁PP | Genotype G and vaccine strains |
| | wherein | Fragment (2-7) |
| | X₁ is Q or R | Consensus sequence |
| 58 | PAIQPP | Vaccine strains and Genotype G |
| | | Genbank ID QFO38283.1 |
| | | Fragment (2-7) |
| 59 | PAIX₁P | Genotype G and vaccine strains |
| | wherein | Fragment (2-6) |
| | X₁ is Q or R | Consensus sequence |
| 60 | PAIQP | Vaccine strains and Genotype G |
| | | Genbank ID QFO38283.1 |
| | | Fragment (2-6) |
| 61 | PAIX₁ | Genotype G and vaccine strains |
| | wherein | Fragment (2-5) |
| | X₁ is Q or R | Consensus sequence |
| 62 | PAIQ | Vaccine strains and Genotype G |
| | | Genbank ID QFO38283.1 |
| | | Fragment (2-5) |
| 63 | PAIQPPLYL TFLLLILLYL IITLYVWIIL | Genotype G Genbank ID |
| | TVTYKTAVRH AALYQRSFFH WSFDHSL | QFO38283.1 |
| | | 2-57 |
| 64 | PAIQPPLYL TFLLLTLLYL IITLYVWTIL | Jeryl Lynn strain or RIT 4385 strain |
| | TINHNTAVRH AALYQRSFSR WGFDQSL | 2-57 |
| 65 | PAIQPPLYP TFLLLILLSL IVTLYVWIIS | Urabe AM9 strain |
| | TITYKTVVRH AALYQRSFFR WSFDHSL | 2-57 |
| 66 | PAIQPPLYL TFLLLILLYL IITLYVWIIL | Leningrad 3 strain 2-57 |
| | TITYKTAVRH AALHQRSFFR WSFDHSL | |
| 67 | PAIQPPLYL TFLLLILLYL IITLYVWIIL | Leningrad-Zagreb strain 2-57 |
| | TITYKTAVRH AALHQRSFFR WSFDHSL | |
| 68 | PAIQPPLYL TFLLLTLLYL IITLYVWTIL | S79 strain 2-57 |
| | TINHNTAVRY AALYQRSFSR WGFDQSL | |
| 69 | PAIQPPLYL TFLLLILLYL IITLYVWTIL | Rubini strain 2-57 |
| | TINHKTAVRY AALYQRSCSR WGFDQSL | |
| 70 | PAIQPPLYP TFLLLILLSL IITLYVWIIS | Hishino strain 2-57 |
| | TITYKTAVRH AALYQRSFFR WSFDHSL | |
| 71 | PAIQPPLHL TFLLLILLYL IITLYVWITL | RS(S-12) strain 2-57 |
| | TITYKTAVRH ATLYQRSFFR WSFDHPL | |
| 72 | PAIQPPLYP TFLLLILLSL IITLYVWIIS | Torii strain 2-57 |
| | TITYKTAVRH ASLYQRSFSR WSFDHSL | |
| 73 | PAIQPPLYP TFLLLILLSL IITLYVWIIS | Mijahada strain 2-57 |
| | TITYKTAVRH AALHQRSFSR WSLDHSL | |
| 74 | MPAIQPPLYL TFLLLILLYL IITLYVWIIL | Genotype G Genbank ID |
| | TVTYKTAVRH AALYQRSFFH WSFDHSL | QFO38283.1 |
| | | Full length |
| 75 | MPAIQPPLYL TFLLLTLLYL IITLYVWTIL | Jeryl Lynn strain |
| | TINHNTAVRH AALYQRSFSR WGFDQSL | Full length |
| 76 | MPAIQPPLYP TFLLLILLSL IVTLYVWIIS | Urabe AM9 strain |
| | TITYKTVVRH AALYQRSFFR WSFDHSL | UniProtKB - P20716 |
| | | Full length |
| 77 | MPAIQPPLYL TFLLLILLYL IITLYVWIIL | Leningrad 3 strain |
| | TITYKTAVRH AALHQRSFFR WSFDHSL | Full length |
| 78 | MPAIQPPLYL TFLLLILLYL IITLYVWIIL | Leningrad-Zagreb strain |
| | TITYKTAVRH AALHQRSFFR WSFDHSL | Full length |
| 79 | MPAIQPPLYL TFLLLTLLYL IITLYVWTIL | S79 strain |
| | TINHNTAVRY AALYQRSFSR WGFDQSL | Full length |
| 80 | MPAIQPPLYL TFLLLILLYL IITLYVWTIL | Rubini strain Full length |
| | TINHKTAVRY AALYQRSCSR WGFDQSL | |
| 81 | MPAIQPPLYP TFLLLILLSL IITLYVWIIS | Hishino strain |
| | TITYKTAVRH AALYQRSFFR WSFDHSL | Full length |
| 82 | MPAIQPPLHL TFLLLILLYL IITLYVWITL | RS(S-12) strain |
| | TITYKTAVRH ATLYQRSFFR WSFDHPL | Full length |
| 83 | MPAIQPPLYP TFLLLILLSL IITLYVWIIS | Torii strain Full length |
| | TITYKTAVRH ASLYQRSFSR WSFDHSL | |
| 84 | MPAIQPPLYP TFLLLILLSL IITLYVWIIS | Mijahada strain |
| | TITYKTAVRH AALHQRSFSR WSLDHSL | Full length |
| 85 | MPAIQPPLYL TFLLL | Genotype G Genbank ID |
| | | QFO38283.1 |
| | | Fragment (1-15) |
| 86 | MPAIQPPLYL TFLL | Genotype G Genbank ID |
| | | QFO38283.1 |
| | | Fragment (1-14) |
| 87 | MPAIQPPLYL TFL | Genotype G Genbank ID |
| | | QFO38283.1 |
| | | Fragment (1-13) |
| 88 | MPAIQPPLYL TF | Genotype G Genbank ID |
| | | QFO38283.1 |
| | | Fragment (1-12) |
| 89 | MPAIQPPLYL T | Genotype G Genbank ID |
| | | QFO38283.1 |
| | | Fragment (1-11) |
| 90 | MPAIQPPLYL | Genotype G Genbank ID |
| | | QFO38283.1 |
| | | Fragment (1-10) |
| 91 | MPAIQPPLY | Genotype G Genbank ID |
| | | QFO38283.1 |
| | | Fragment (1-9) |
| 92 | PAIQPPLYL TFLLL | Genotype G Genbank ID |
| | | QFO38283.1 |
| | | Fragment (2-15) |
| 93 | PAIQPPLYL TFLL | Genotype G Genbank ID |
| | | QFO38283.1 |
| | | Fragment (2-14) |
| 94 | PAIQPPLYL TFL | Genotype G Genbank ID |
| | | QFO38283.1 |
| | | Fragment (2-13) |
| 95 | PAIQPPLYL TF | Genotype G Genbank ID |
| | | QFO38283.1 |
| | | Fragment (2-12) |
| 96 | PAIQPPLYL T | Genotype G Genbank ID |
| | | QFO38283.1 |
| | | Fragment (2-11) |
| 97 | PAIQPPLYL | Genotype G Genbank ID |
| | | QFO38283.1 |
| | | Fragment (2-10) |
| 98 | PAIQPPLY | Genotype G Genbank ID |
| | | QFO38283.1 |
| | | Fragment (2-9) |
| 115 | MPAIQPPLYL TELLLILLYL IITLYVWIIL | Genotype G Genbank ID |
| | TITYKTAVRH AALYQRSFFR WSFDHSL | QFO38283.1 1-57 |
| 116 | PAIQPPLYLTFLLLI | Genotype G Genbank ID |
| | | QFO38283.1 2-16 |
| 117 | PAIQPPLYLTFLLLIL | Genotype G Genbank ID |
| | | QFO38283.1 2-17 |
| 118 | PAIQPPLYLTFLLLILL | Genotype G Genbank ID |
| | | QFO38283.1 2-18 |
| 119 | PAIQPPLYLTFLLLILLY | Genotype G Genbank ID |
| | | QFO38283.1 2-19 |
| 120 | PAIQPPLYLTFLLLILLYL | Genotype G Genbank ID |
| | | QFO38283.1 2-20 |
| 121 | PAIQPPLYLTFLLLILLYLI | Genotype G Genbank ID |
| | | QFO38283.1 2-21 |
| 122 | MPAIQPPLYLTFLLLI | Genotype G Genbank ID |
| | | QFO38283.1 1-16 |
| 123 | MPAIQPPLYLTFLLLIL | Genotype G Genbank ID |
| | | QFO38283.1 1-17 |
| 124 | MPAIQPPLYLTFLLLILL | Genotype G Genbank ID |
| | | QFO38283.1 1-18 |
| 125 | MPAIQPPLYLTFLLLILLY | Genotype G Genbank ID |
| | | QFO38283.1 1-19 |
| 126 | MPAIQPPLYLTFLLLILLYL | Genotype G Genbank ID |
| | | QFO38283.1 1-20 |
| 127 | HAEGTFTSDV SSYLEGQAAK | GLP-1 7-36 / GLP-1 7-36 amide |
| | EFIAWLVKGR | |
| 128 | See FIG. 12 | SH(2-11) (PAIQPPLYLT) protein |
| | SH(2-11)-GLP-1(7-36) | connected via C-terminal residue |
| | conjugate. | and PEG2-spacer to Lys26 of GLP- |
| | | 1 (7-36) |
| 129 | — | TAMRA-functionalized SH protein |
| 130 | HGEGTFTSDL SKQMEEEAVR | Exendin-4 (1-39) |
| | LFIEWLKNGG PSSGAPPPS | |
| 131 | See FIG. 13 | SH(2-11) (PAIQPPLYLT) protein |
| | SH(2-11)-Exendin-4(1-39) | connected via C-terminal residue |
| | conjugate. | and PEG2-spacer to Lys 27 of |
| | | Exendin-4 (1-39) |
| 132 | HAEGTFTSDV SSYLEGQAAK | GLP-1 7-37 |
| | EFIAWLVKGR G | |

### REFERENCES

Coca-Prados, M. (2014). The blood-aqueous barrier in health and disease. J Glaucoma, 23(8 Suppl 1), S36-38.
Deacon et al, Journal of Endocrinology; 2002; 172; 355-362
Gutzman, J. H., & Sive, H. (2009). Zebrafish brain ventricle injection. J Vis Exp(26).
He, Z., Kokkinaki, M., Jiang, J., Zeng, W., Dobrinski, I., & Dym, M. (2012). Isolation of human male germ-line stem cells using enzymatic digestion and magnetic-activated cell sorting. Methods Mol Biol, 825, 45-57.
Henson, H. E., Parupalli, C., Ju, B., & Taylor, M. R. (2014). Functional and genetic analysis of choroid plexus development in zebrafish. Front Neurosci, 8, 364.
Li, X., Roszko, I., Sepich, D. S., Ni, M., Hamm, H. E., Marlow, F. L., & Solnica-Krezel, L. (2013). Gpr125 modulates Dishevelled distribution and planar cell polarity signaling. Development, 140(14), 3028-3039.
Petersen, N., Torz, L., Jensen, K. H. R., Hjorto, G. M., Spiess, K., & Rosenkilde, M. M. (2020). Three-Dimensional Explant Platform for Studies on Choroid Plexus Epithelium. Front Cell Neurosci, 14, 108.
Pickering, C., Hagglund, M., Szmydynger-Chodobska, J., Marques, F., Palha, J. A., Waller, L., Chodobski, A., Fredriksson, R., Lagerstrom, M. C., & Schioth, H. B. (2008). The Adhesion GPCR GPR125 is specifically expressed in the choroid plexus and is upregulated following brain injury. BMC. Neurosci, 9, 97.
Seandel, M., Falciatori, I., Shmelkov, S. V., Kim, J., James, D., & Rafii, S. (2008). Niche players: spermatogonial progenitors marked by GPR125. Cell Cycle, 7(2), 135-140.
Spiess, K., Bagger, S. O., Torz, L. J., Jensen, K. H. R., Walser, A. L., Kvam, J. M., Mogelmose, A. K., Daugvilaite, V., Junnila, R. K., Hjorto, G. M., & Rosenkilde, M. M. (2019). Arrestin-independent constitutive endocytosis of GPR125/ADGRA3. Ann. N. Y. Acad. Sci, 1456(1), 186-199.
Spina, E. H., R.; Simondza, J.; Incassati, A.; Faiq, M.; Sainulabdeen, A.; Chan, K. C.; Cowin, P.; . (2021). Gpr125 identifies myoepithelial progenitors at tips of lacrimal ducts and is essential for tear film. bioRXiv.
Westerfield, M. (2000). The Zebrafish Book. A Guide for the Laboratory Use of Zebrafish (Danio rerio) (4 ed.). University of Oregon Press).
Woods, D. F., Hough, C., Peel, D., Callaini, G., & Bryant, P. J. (1996). Dlg protein is required for junction structure, cell polarity, and proliferation control in Drosophila epithelia. J Cell Biol, 134(6), 1469-1482.
Woznik, M., Rodner, C., Lemon, K., Rima, B., Mankertz, A., & Finsterbusch, T. (2010). Mumps virus small hydrophobic protein targets ataxin-1 ubiquitin-like interacting protein (ubiquilin 4). J. Gen. Virol, 91(Pt 11), 2773-2781.
Xu, H., Yang, M., Tian, R., Wang, Y., Liu, L., Zhu, Z., Yang, S., Yuan, Q., Niu, M., Yao, C., Zhi, E., Li, P., Zhou, C., He, Z., Li, Z., & Gao, W. Q. (2020). Derivation and propagation of spermatogonial stem cells from human pluripotent cells. Stem Cell Res Ther, 11(1), 408.

### Examples

### Example 1: GPR125 Knock Down (KD) Mouse Model Using AAV System

The aim of the present example was to investigate change in gene expression when GPR125 is knock down and validate potential importance in membrane structure and/or permeability

### Materials and Methods:

Animals were maintained on a 12-h light/dark cycle and had ad libitum access to water and a chow diet (Altromin no. 1314; Brogaarden) unless otherwise stated. The studies in mice were approved by the Danish Animals Inspectorate and were performed according to institutional guidelines.

4-5 weeks old male C57BI/6 (Scanbur —Charles River) underwent stereotaxic surgery to inject 0.5 μl in each lateral ventricle (2 injections in total) of either 3.3×10¹¹ GC/ml AAV5-mCherry-U6-ADRAG3-shRNA to knock down (KD) GPR125 or 1.6×10¹² GC/ml AAV5-mCherry-U6-scrmb-shRNA as control.

Male mice were anesthetized with 4-5% isoflurane for induction and 1-2% for maintenance and inject 10 ml/kg BW carprofen s.c. The mouse was fixed in a stereotaxic instrument. Fixate mouse in stereotaxic frame by placing metal bars right in front of the ears. A cream was applied on the eyes to prevent drying and a drop of marcain and bupivacaine was injected under the skin of the skull. Subsequently, the scalp is cut open with a scalpel from right behind the eyes and approx. 1-1.5 cm back. The skin is pushed to the sides with Q-tips, the skull is dried. Then check that bregma and lambda are aligned, which allow to calculate the exact location for drilling.

Two separate holes were drilled at the wanted anterior-posterior (AP) and medial-lateral (ML) coordinates. The Hamilton syringe was placed to align with the skull surface at the expected coordinates, and the needle was slowly inserted into the brain in one of the Y positions and lower until the desired Z coordinate. Virus was injected over 5 min into each injection site. The skin on the skull was sutured together and the mouse was placed in a new heated cage. The mice were treated with carprofen (10 ml/kg BW) s.c. for two days afterwards and watched carefully.

At 18-23 weeks of age, AAV injected males were euthanized and CP were collected and stored in RNA later for 24 hours at 4ºC. Subsequently, RNA later was removed and the CP were stored at −80ºC until further analysis. RNA was extracted using RNeasy micro kit (Qiagen). RNA was used either for RNAseq without further treatment or for qPCR (SYBR green) after a reverse transcriptase PCR (Superscript III, reverse transcriptase, Thermo Fisher). The list of the primer used is shown in table 1. RNA was sequenced by Novogene using Novaseq 600 (2 sequencing lanes, paired-end, 150 reads, low-input library) and further control of the samples was done as following: mapping software: hisat2; version: hisat22.0.5; parameters: hisat2 -p 4 —no-unal -t —phred33 —dta-cufflinks.

DGE software: DESeq2; version: DE_analysis.v2.py; parameters: DE_analysis.v2.py-m DESeq2 —padjust 0.05 —foldchange 1.

TABLE 1

| Primer ID | Forward | Reverse |
|---|---|---|
| GPR125v | ttt ctg act atg ggc gaa gg | gcc tcc atg atg gtg ttc tt |
| 2 | (SEQ ID NO: 99) | (SEQ ID NO: 100) |
| GPR 125pi | atg ctt gtg aac ctg tgc ttt c | cgc tgg cat ttc tgg tct gg′ |
| ck | (SEQ ID NO: 101) | (SEQ ID NO: 102) |
| YWHAZ | aga cgg aag gtg ctg aga aa | gaa gca ttg ggg atc aag aa |
| | (SEQ ID NO: 103) | (SEQ ID NO: 104) |
| clu | ATGAAGATTCTCCTGCTGCG | CTTCACCACCACCTCAGTG |
| | (SEQ ID NO: 105) | (SEQ ID NO: 106) |
| Ttr | AGCCCTTTGCCTCTGGGAAGAC | TGCGATGGTGTAGTGGCGAT |
| | (SEQ ID NO: 107) | GG |
| | | (SEQ ID NO: 108) |
| Ptgds | Gggaatcccaagagacccag | Gctctgagcaaatggctgc |
| | (SEQ ID NO: 109) | (SEQ ID NO: 110) |
| MMP-9 | AAGGACGGCCTTCTGGCACACG | GTGGTATAGTGGGACACATA |
| | CCTTT | GTGG |
| | (SEQ ID NO: 111) | (SEQ ID NO: 112) |
| occludin | ATGACTTCAGGCAGCCTCGTTAC | TCAGCAGCAGCCATGTACTCT |
| | A | TCA |
| | (SEQ ID NO: 113) | (SEQ ID NO: 114) |

### Results:

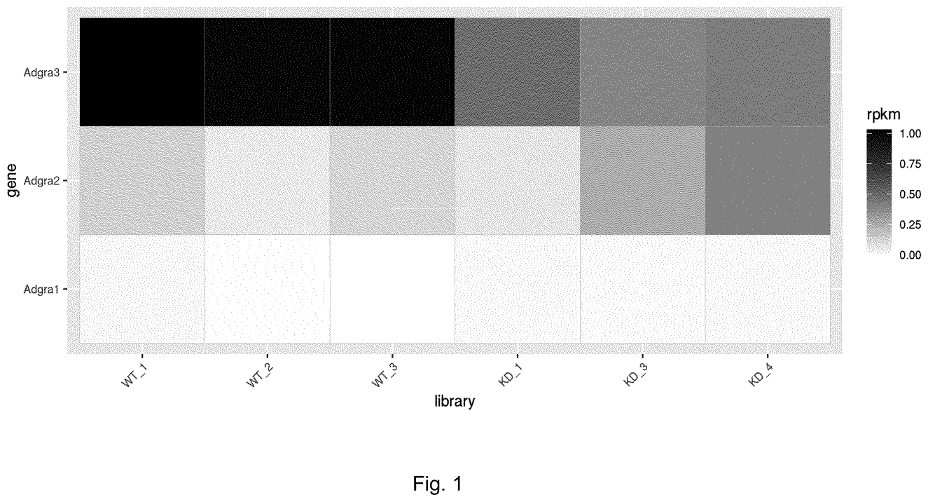

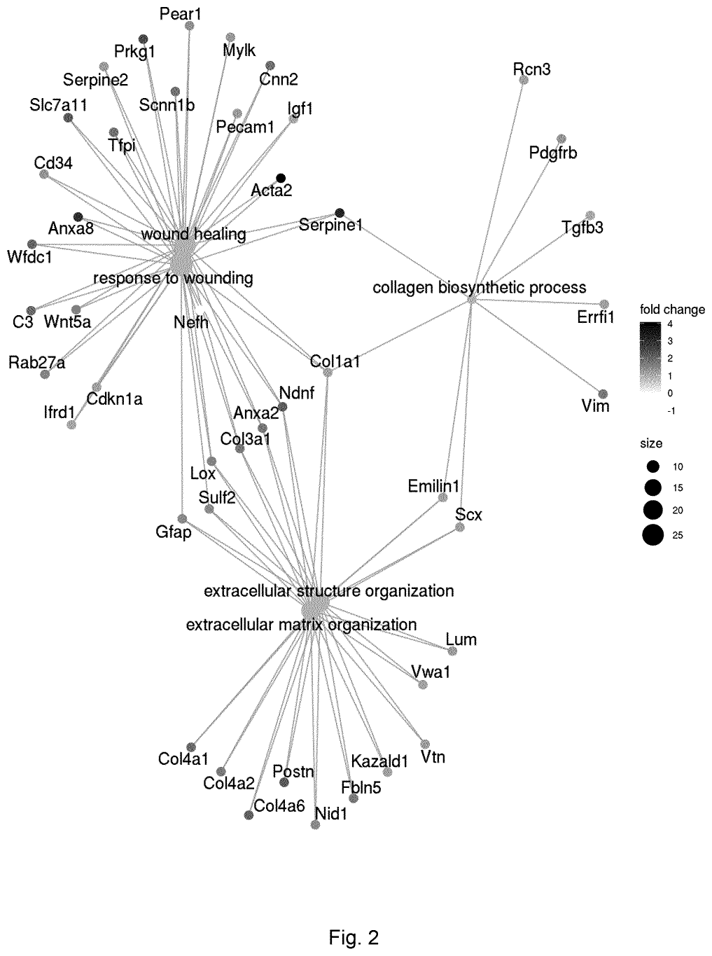

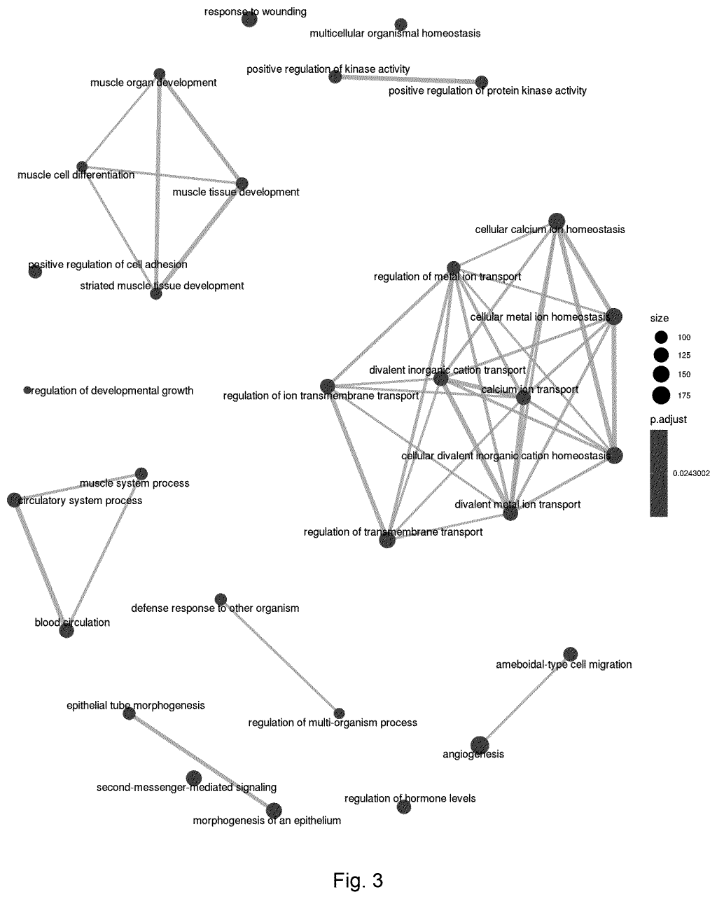

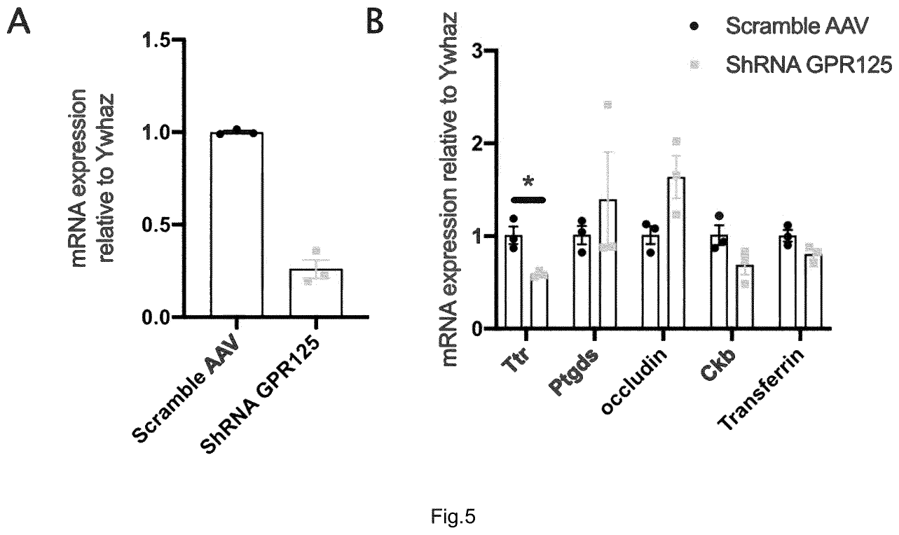

RNAseq data confirmed that GPR125 is highly expressed in the BCSFB as expected (Pickering 2008) (FIG. 1). RNA seq data further showed that the receptor is important for wound healing, extracellular matrix organization as well as for the morphogenesis of the epithelium (FIG. 2 and FIG. 3), supporting its role in the barrier function of the choroid plexus. GPR125 further influences the expression or is co-acting with transporters, like transferrin and transthyretin to control the transport of molecules through epithelium (FIG. 5). In addition, GPR125 plays a role in cell adhesion molecule expression and leads to an increase of tight junction genes like claudin (Cldn 1, table 3) and adherens junction genes like cadherin (Cdh 1, table 3). GPR125 KD induces overexpression of extracellular matrix signals like collagen and laminin (Col2a1 and Lama1, Table 2). Moreover, GPR125 is involved in membrane permeability and transport and knocking it down leads to increase aquaporin 2, essential for water transport (Aqp2, Table 5), sodium/hydrogen exchanger 3 (SLC9A3, Table 4) and duodenal cytochrome b (Cybrd1, table 4), for sodium and ferric ion transport respectively, and ATP-binding cassette ABC transporter (e.g., Abcb11, Abcc12 and Abcg3, Table 2).

TABLE 2

| ABC Transporter | | Focal adhesion | | ECM-receptor interaction | |
|---|---|---|---|---|---|
| Gene | | Gene | | Gene | |
| name | log2FC | name | log2FC | name | log2FC |
| Abca1 | 0.0854 | Actb | 0.3860 | Agrn | 0.1784 |
| Abca12 | NA | Actn4 | 0.2995 | Cd36 | 0.0631 |
| Abca13 | 1.1983 | Akt1 | −0.4090 | Cd44 | 0.4248 |
| Abca2 | −0.0800 | Arhgap5 | −0.0785 | Cd47 | 0.0767 |
| Abca3 | 0.2823 | Bad | −0.0257 | Chad | 1.8597 |
| Abca4 | −0.1680 | Bcar1 | −0.1526 | Col2a1 | 14.0238 |
| Abca5 | 0.3396 | Bcl2 | −0.3881 | Comp | 1.1577 |
| Abca6 | −0.0051 | Birc3 | 0.3483 | Dag1 | 0.0512 |
| Abca7 | −0.4588 | Braf | −0.3805 | Dmp1 | 2.2078 |
| Abca8b | −1.1818 | Capn2 | 0.1931 | Dspp | NA |
| Abca9 | 1.3450 | Cav1 | 2.4610 | Fn1 | 1.5001 |
| Abcb10 | 0.0581 | Ccnd1 | 1.0798 | Fras1 | −0.3150 |
| Abcb11 | 5.0926 | Cdc42 | 0.1103 | Frem2 | 0.7524 |
| Abcb1b | 2.7339 | Chad | 40.5177 | Gp1ba | −1.1384 |
| Abcb4 | −0.4983 | Crk | 0.4877 | Gp1bb | 0.7013 |
| Abcb5 | NA | Ctnnb1 | 0.0088 | Gp5 | 1.7711 |
| Abcb6 | −0.2964 | Diaph1 | −0.2631 | Gp6 | 6.0638 |
| Abcb7 | −0.0693 | Dock1 | −0.2281 | Gp9 | 1.1507 |
| Abcb8 | −0.0235 | Egf | 2.0913 | Hmmr | −5.5669 |
| Abcb9 | −0.5011 | Egfr | 3.8186 | Hspg2 | −0.0434 |
| Abcc1 | −0.2758 | Elk1 | −0.1940 | Ibsp | NA |
| Abcc10 | −0.1777 | Flnc | 1.3846 | Itga1 | 0.0256 |
| Abcc12 | −5.8588 | Fyn | 0.3871 | Itga10 | 0.1914 |
| Abcc2 | NA | Grb2 | 0.1115 | Itga11 | −0.9232 |
| Abcc3 | −1.6204 | Gsk3b | 0.0674 | Itga2 | 1.1597 |
| Abcc4 | −0.0204 | Hras | −0.3393 | Itga2b | −0.3224 |
| Abcc5 | 0.0071 | Ilk | −0.0620 | Itga3 | −0.2180 |
| Abcc6 | 1.1553 | Itga2 | 1.2296 | Itga4 | −0.2674 |
| Abcc8 | −0.1096 | Itgb1 | 3.7117 | Itga5 | 0.1853 |
| Abcc9 | 1.5846 | Jun | 0.8758 | Itga6 | 0.2981 |
| Abcd1 | 0.6284 | Map2k1 | 0.2916 | Itga7 | 1.0532 |
| Abcd2 | 1.0999 | Mapk1 | 0.1202 | Itga8 | 0.8230 |
| Abcd3 | −0.0358 | Mapk10 | 0.1922 | Itga9 | −0.5994 |
| Abcd4 | 0.3572 | Myl7 | 6.8833 | Itgav | −0.1760 |
| Abcg1 | −0.1199 | Mylk | 1.3426 | Itgb1 | 0.1725 |
| Abcg2 | 0.1552 | Pak1 | −0.4808 | Itgb3 | 1.4026 |
| Abcg3 | −5.8148 | Parva | −0.3383 | Itgb4 | 0.6202 |
| Abcg4 | 0.0052 | Pdpk1 | 0.0265 | Itgb5 | −0.0851 |
| Abcg5 | 0.0410 | Pik3ca | −1.7686 | Itgb6 | 0.2485 |
| Abcg8 | 2.3481 | Pip5k1c | −0.0160 | Itgb7 | 1.0872 |
| Cftr | 7.4910 | Ppp1r12a | 1.6416 | Itgb8 | 0.2657 |
| Defb1 | 2.7939 | Prkca | 0.3967 | Lama1 | 14.6286 |
| Tap1 | 0.2075 | Pten | −0.0302 | Npnt | −0.3215 |
| Tap2 | 0.4625 | Ptk2 | −0.0211 | Reln | 1.6862 |
| | | Pxn | −0.2237 | Sdc1 | 0.0547 |
| | | Rac1 | −0.0997 | Spp1 | 1.7817 |
| | | Raf1 | −0.0747 | Sv2a | −0.1646 |
| | | Rap1a | 0.3365 | Tnc | −1.4913 |
| | | Rapgef1 | 0.1624 | Vtn | 1.2328 |
| | | Rasgrf1 | 0.2650 | Vwf | 4.1383 |
| | | Rhoa | 0.0226 | | |
| | | Rock1 | 0.3487 | | |
| | | Shc1 | 1.7465 | | |
| | | Sos1 | −0.4217 | | |
| | | Src | 0.0737 | | |
| | | Tln1 | 0.6706 | | |
| | | Vasp | −0.0281 | | |
| | | Vav1 | 0.3446 | | |
| | | Vcl | 0.7468 | | |
| | | Zyx | −0.4953 | | |

TABLE 3

| Cell adhesion | | Adherens | | | |
|---|---|---|---|---|---|
| molecules | | junction | | Tight junction | |
| Gene | | Gene | | Gene | |
| name | log2FC | name | log2FC | name | log2FC |
| Alcam | 2.0785 | Acp1 | 0.0456 | Actb | 0.3860 |
| Cadm1 | 0.7966 | Actb | 0.3860 | Actn4 | 0.2995 |
| Cadm3 | 0.0125 | Actn4 | 0.2995 | Actr2 | −0.4274 |
| Cd2 | −0.4761 | Afdn | 0.1845 | Afdn | 0.1845 |
| Cd22 | −3.0743 | Baiap2 | 0.0043 | Amot | 0.9279 |
| Cd226 | 0.5519 | Cdc42 | 0.1103 | Arhgap17 | −0.4826 |
| Cd274 | 0.1853 | Cdh1 | 2.0014 | Arhgef18 | −0.3927 |
| Cd276 | −0.4618 | Crebbp | −0.5581 | Arhgef2 | 0.0051 |
| Cd28 | −4.5382 | Csnk2a1 | −0.4953 | Bves | −4.1803 |
| Cd34 | 1.3278 | Ctnna1 | −1.3023 | Cacna1d | 0.5515 |
| Cd4 | 0.7884 | Ctnnb1 | 0.0088 | Ccnd1 | 0.7404 |
| Cd40 | 0.4779 | Ctnnd1 | −0.0671 | Cd1d1 | −0.2250 |
| Cd40lg | 4.9489 | Egfr | −0.2311 | Cdc42 | 0.1103 |
| Cd6 | 6.7578 | Farp2 | −0.0166 | Cdk4 | −0.0216 |
| Cd80 | 0.2944 | Fer | 0.2054 | Cftr | 7.4910 |
| Cd86 | −0.6578 | Fgfr1 | −0.0486 | Cgn | −0.3645 |
| Cd8a | 0.1905 | Fyn | 0.3871 | Cgnl1 | −0.3179 |
| Cd99l2 | 0.1106 | Igf1r | −0.3289 | Cldn1 | 4.8430 |
| Cdh1 | 2.0014 | Insr | 0.0874 | Crb3 | 0.1245 |
| Cdh15 | −3.1307 | Iqgap1 | −0.0254 | Cttr | 0.0400 |
| Cdh2 | −0.0554 | Lef1 | 1.1051 | Dlg1 | −0.0778 |
| Cdh3 | 0.0301 | Lmo7 | 1.0433 | Dlg2 | 0.8118 |
| Cdh4 | −0.8142 | Map3k7 | 0.1453 | Dlg3 | −0.1580 |
| Cdh5 | 1.8499 | Mapk1 | 0.1202 | Epb41l4b | 0.5689 |
| Cldn1 | 4.8430 | Met | −0.4838 | Erbb2 | −0.2179 |
| Cntn2 | −0.0174 | Nectin2 | 0.6311 | F11r | −0.2746 |
| Cntnap1 | −0.3551 | Nlk | 0.2910 | Gata4 | NA |
| Cntnap2 | 0.4611 | Pard3 | 0.3533 | Hspa4 | 0.0747 |
| Ctla4 | NA | Ptpn1 | 0.0228 | Igsf5 | −0.1381 |
| Esam | 1.2629 | Ptpn6 | −0.2922 | Itgb1 | 0.1725 |
| F11r | −0.2746 | Ptprb | 1.6478 | Jun | 0.8758 |
| Glg1 | 0.0867 | Ptprf | −0.3290 | Llgl1 | −0.4243 |
| Glycam1 | 1.8773 | Ptprj | 0.3300 | Magi1 | 0.2223 |
| Gm7030 | 0.9619 | Ptprm | 0.3130 | Map2k7 | −0.1025 |
| H2-Aa | NA | Rac1 | −0.0997 | Map3k1 | 0.1136 |
| Icam1 | −0.0479 | Rhoa | 0.0226 | Map3k5 | −0.5005 |
| Icam2 | 1.1278 | Smad3 | 0.0758 | Mapk10 | 0.1922 |
| Icos | NA | Smad4 | 0.1206 | Marveld2 | −0.0667 |
| Icosl | −0.2782 | Snai1 | 1.3531 | Marveld3 | −0.0868 |
| Igsf11 | 0.2892 | Snai2 | 0.3784 | Micall2 | 0.2119 |
| Itga4 | −0.2674 | Sorbs1 | −0.0736 | Mpdz | −0.0595 |
| Itga6 | 0.2981 | Src | 0.0737 | Mpp4 | −1.0288 |
| Itga8 | 0.8230 | Ssx2ip | −0.2838 | Mpp5 | 0.0169 |
| Itga9 | −0.5994 | Tgfbr1 | 0.1952 | Msn | 0.4489 |
| Itgal | 0.7064 | Tjp1 | 0.9226 | Myh11 | 18.9991 |
| Itgam | 0.8878 | Vcl | 0.7468 | Myl2 | −0.5394 |
| Itgav | −0.1760 | Was | 0.8275 | Nedd4 | 1.2102 |
| Itgb1 | 0.1725 | Wasf1 | 0.8537 | Nf2 | −0.1841 |
| Itgb2 | 1.2735 | Wasl | −0.0739 | Ocln | 0.2388 |
| Itgb7 | 1.0872 | Yes1 | −0.5276 | Pard3 | 0.3533 |
| Itgb8 | 0.2657 | | | Pard6a | 0.6682 |
| Jam2 | 0.6744 | | | Patj | −0.6118 |
| Jam3 | 0.7327 | | | Pcna | −0.1680 |
| L1cam | 1.1136 | | | Ppp2ca | 0.7216 |
| Lrrc4 | 0.8847 | | | Prkab1 | 0.6334 |
| Lrrc4b | 0.8506 | | | Prkaca | −0.0021 |
| Lrrc4c | −1.2579 | | | Prkce | 0.6804 |
| Madcam1 | 0.3932 | | | Prkci | 0.1777 |
| Mag | 0.7264 | | | Rab13 | 0.4625 |
| Mpz | 0.4104 | | | Rab8a | −0.1426 |
| Ncam1 | 1.6824 | | | Rac1 | 0.1903 |
| Nectin1 | −1.2115 | | | Rap1a | 0.1570 |
| Nectin2 | 0.2084 | | | Rap2c | 0.3119 |
| Nectin3 | 0.1525 | | | Rapgef2 | 0.0453 |
| Negr1 | 0.1755 | | | Rapgef6 | −0.0359 |
| Neo1 | 0.1793 | | | Rhoa | 0.0226 |
| Nfasc | −0.6164 | | | Rhoa | 0.0226 |
| Nlgn1 | 0.4376 | | | Rock1 | 0.3487 |
| Nrcam | 0.4424 | | | Runx1 | −0.1357 |
| Nrxn1 | 1.2690 | | | Scrib | −0.2926 |
| Ntng1 | 2.3197 | | | Slc9a3r1 | −0.1884 |
| Ntng2 | −1.0131 | | | Src | 0.0737 |
| Ocln | 0.2388 | | | Stk11 | −0.0140 |
| Pdcd1 | −4.9649 | | | Sympk | −0.4323 |
| Pdcd1lg2 | NA | | | Synpo | 0.5310 |
| Pecam1 | 1.2593 | | | Tiam1 | −0.8050 |
| Ptprc | 1.0514 | | | Tjap1 | −0.1653 |
| Ptprf | −0.3290 | | | Tjp1 | 0.9226 |
| Ptprm | 0.3130 | | | Tjp3 | −0.1730 |
| Pvr | −0.1117 | | | Tuba1a | −2.1043 |
| Sdc2 | 0.8264 | | | Vasp | −0.0281 |
| Sele | 3.0551 | | | Was | 0.6665 |
| Sell | 1.4095 | | | Was | 0.6665 |
| Selp | 2.0297 | | | Ybx3 | 0.5099 |
| Selplg | 0.5162 | | | | |
| Siglec1 | 2.3565 | | | | |
| Spn | −0.5178 | | | | |
| Tigit | NA | | | | |
| Vcam1 | −0.6582 | | | | |
| Vcan | 0.8273 | | | | |
| Vsir | 0.6717 | | | | |
| Vtcn1 | NA | | | | |

TABLE 4

| Reg of actin | | | | | |
|---|---|---|---|---|---|
| cytoskeleton | | Gap junction | | Mineral absorption | |
| Gene | | Gene | | Gene | |
| name | log2FC | name | log2FC | name | log2FC |
| Abi2 | −0.0361 | Adcy6 | 5.4921 | Atox1 | −0.3785 |
| Actb | 0.3860 | Adrb1 | 0.5510 | Atp1a1 | −0.9729 |
| Actn4 | 0.2995 | Cdk1 | 0.2362 | Atp2b2 | −0.8753 |
| Apc | −0.4090 | Csnk1d | −0.0515 | Atp7a | 0.1218 |
| Araf | −0.4275 | Drd1 | −3.4479 | Clcn2 | 0.0671 |
| Arhgap35 | −0.1270 | Drd2 | −3.7417 | Cybrd1 | 1.3565 |
| Arhgef1 | −0.2032 | Egf | −1.6077 | Fth1 | −0.0113 |
| Arhgef6 | −0.3445 | Egfr | 2.1340 | Heph | −0.6205 |
| Arhgef7 | 0.0405 | Gja1 | −2.5711 | Hmox1 | 0.0428 |
| Arpc1b | 0.1496 | Gna11 | 0.6266 | Mt1 | 4.1693 |
| Baiap2 | 0.0043 | Gnai1 | 0.0721 | S100g | −0.8439 |
| Bcar1 | −0.1526 | Gnas | −0.0417 | Slc11a2 | −0.2311 |
| Bdkrb1 | 5.1790 | Grb2 | 0.1115 | Slc26a3 | −0.4725 |
| Brk1 | 0.1191 | Grm1 | 0.4254 | Slc26a6 | −0.1765 |
| Cdc42 | 0.1103 | Gucy1b1 | −0.9515 | Slc26a9 | NA |
| Cfl1 | 0.2339 | Hras | −0.6170 | Slc30a1 | 0.4249 |
| Crk | 0.4877 | Htr2a | −0.0730 | Slc31a1 | −0.2817 |
| Cxcl12 | 1.1589 | Itpr1 | 1.0304 | Slc34a1 | NA |
| Cxcr4 | 0.7863 | Lpar1 | 1.5101 | Slc39a4 | 0.3354 |
| Cyfip1 | −0.0983 | Map2k1 | 0.1254 | Slc40a1 | 0.2962 |
| Diaph1 | −0.2747 | Map2k5 | 0.1505 | Slc46a1 | −0.2100 |
| Diaph3 | −0.0389 | Map3k2 | 0.1381 | Slc5a1 | −0.3292 |
| Dock1 | −0.2281 | Mapk1 | 0.1202 | Slc6a19 | −0.1949 |
| Egf | 4.2988 | Mapk7 | 0.0364 | Slc8a1 | 0.6722 |
| Egfr | −3.6876 | Plcb1 | 0.9408 | Slc9a3 | 5.0778 |
| Enah | 0.02223 | Prkaca | −0.0021 | Steap1 | −0.2073 |
| F2 | NA | Prkca | 0.3967 | Trf | −0.3973 |
| F2r | 0.9760 | Prkg1 | 4.3009 | Trpm7 | 0.6473 |
| Fgd1 | 0.8402 | Raf1 | −0.0747 | Trpv6 | NA |
| Fn1 | 1.5001 | Sos1 | −0.4217 | Vdr | 1.9719 |

| | | | |
|---|---|---|---|
| Git1 | −0.1743 | Src | 0.0737 |
| Gna12 | −0.0041 | Tjp1 | 0.9226 |
| Gng12 | 4.6958 | Tuba1a | −2.1042 |
| Hras | −0.6672 | Tubb2a | 3.5641 |

| | |
|---|---|
| Ins1 | 0.7564 |
| Iqgap1 | 0.2837 |
| Itga2 | 9.4338 |
| Kng2 | 0.2280 |
| Limk1 | 0.1613 |
| Lpar1 | 6.7988 |
| Map2k1 | 0.1254 |
| Mapk1 | 0.1202 |
| Msn | 0.4489 |
| Myh11 | 2.3991 |
| Myl7 | 6.8833 |
| Mylk | 1.3426 |
| Nckap1 | −0.8565 |
| Pak1 | −0.4808 |
| Pfn1 | −0.6599 |
| Pik3ca | −1.7686 |
| Pikfyve | 0.0571 |
| Ppp1r12a | 1.6416 |
| Ptk2 | −0.0211 |
| Pxn | −0.2237 |
| Rac1 | −0.0997 |
| Rhoa | 0.0226 |
| Rock1 | 0.3487 |
| Scin | 6.2760 |
| Slc9a1 | 0.0902 |
| Sos1 | −0.4217 |
| Spata13 | 0.9778 |
| Src | 0.0737 |
| Ssh1 | −0.7706 |
| Tiam1 | −0.4604 |
| Tmsb4x | 0.8392 |
| Vcl | 0.7468 |
| Wasf1 | 0.2039 |
| Wasf2 | 0.2124 |
| Wasl | −0.0739 |

TABLE 5
Vasop-reg water reabsorption

| Gene | |
|---|---|
| name | log2FC |
| Adcy6 | 2.0886 |
| Aqp2 | 2.2343 |
| Aqp3 | NA |
| Aqp4 | 1.0291 |
| Arhgdib | 0.8192 |
| Avp | NA |
| Avpr2 | NA |
| Creb1 | −2.8610 |
| Dctn1 | 0.0643 |
| Dync1h1 | −3.8305 |
| Gnas | −0.0417 |
| Nsf | −0.1398 |
| Prkaca | −0.0021 |
| Rab11b | −0.1211 |
| Rab5b | 0.1783 |
| Stx4a | −0.1296 |
| Vamp2 | −0.0265 |

### Conclusion:

This example demonstrates that GPR125 is important for membrane structure and permeability.

### Example 2: Zebrafish

Aim: The aim of this example was to study whether lack of GPR125 in zebrafish changes the barriers into the brain.

### Materials and Methods:

Danio rerio embryos of the wildtype AB-line were raised according to standard laboratory conditions (Westerfield, 2000). GPR125 knock out (KO) AB* embryos were generated by injecting antisense morpholino oligonucleotides blocking translation or splicing of adgra3 were into one-cell stage embryos (Li et al., 2013). The studies in zebrafish were approved by the Danish Animals Inspectorate and were performed according to institutional guidelines.

### Brain Ventricle Size by Dye-injection

WT (n=7) and KO (n=7) embryos were fertilized at the same time by using a glass separator to ensure coitus was simultaneous. Phenylthiourea (PTU) was added 20 hours post fertilization (hpf) to prevent pigmentation.

At 28 hpf the fish were anaesthetized with 200 μg/ml Tricaine, placed in an agar well with the embryo tail down into the wells and positioned so the brain and ventricles were visible from above and the hind brain ventricle was accessible. 4% 10 kDa dextran-TAMRA in 0.2M KCl was loaded into the glass needle and 2-4 nl of the dye was injected into the hindbrain ventricle of the embryo (Gutzman & Sive, 2009).

The embryos were imaged approximately an hour later in a fluorescent stereomicroscope with a red filter. The width of the ventricles and the diameter of the eye and yoke sack was measured.

### Choroid Plexus Permeability by Dye-Injection

WT (n=11) and KO (n=11) embryos fertilized at the same time and PTU-treated 20 hpf were anaesthetized 4 days post fertilization (dpf). They were placed belly down on a ridge of agar. 4 nl of a mixture of 1% 3 kDa dextran-BrilliantBlue, 1% 10 kDa dextran-TAMRA, 1% 40 kDa dextran-Alexa488 in 0,2% KCL was injected into the a. communis of the fish and incubated for 30 min. Then fixed in 4% PFA overnight at 4C.

Placed on the side, the fish were imaged with a confocal LSM 700 (Zeiss) using a EC Plan-Neofluar 10x/0.30 M27 objective. A Z-stack of up to 12 images of the head and upper body was taken for each dye. A maximum intensity projection of the Z-stack was made and the ratio of fluorescence intensity in the brain ventricles relative to the heart was measured at 30 min post-injection as a readout for tracer leakage into the brain ventricle (Henson et al., 2014).

### Results:

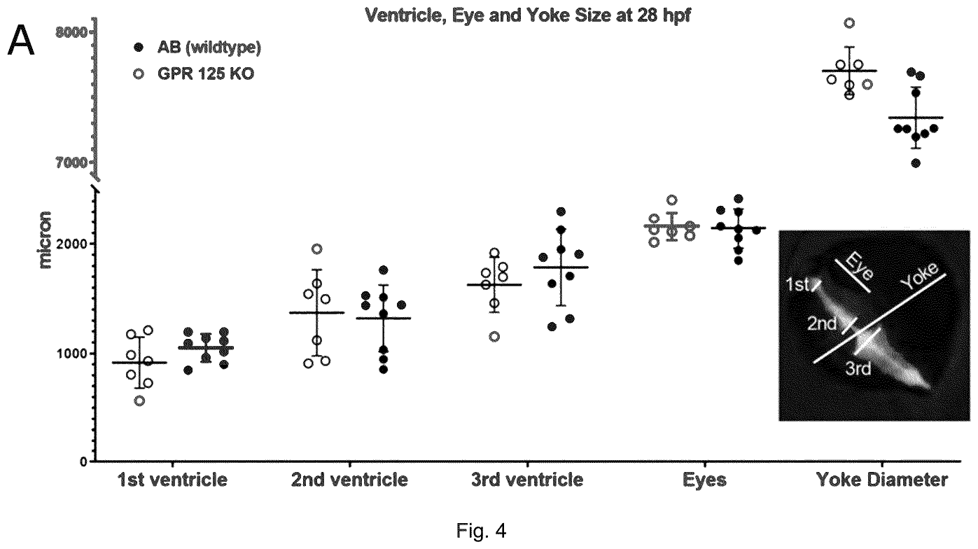

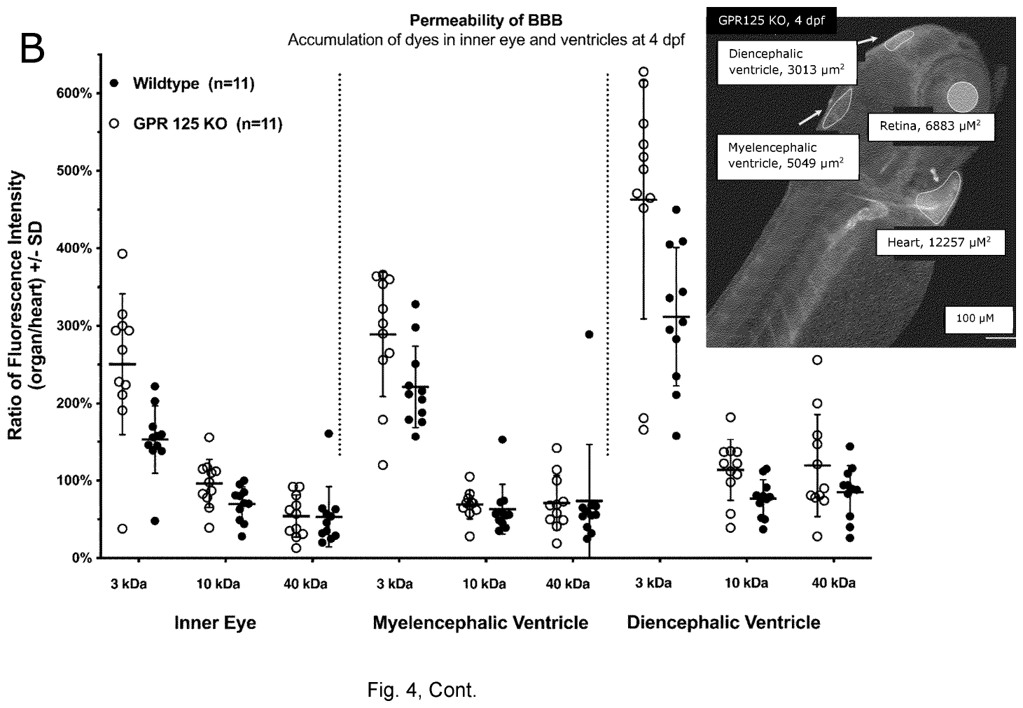

We further confirmed the role of GPR125 for keeping the BCSFB tight by knocking out the receptor in zebrafish, which led to a leakiness of the blood brain barriers performing permeability studies injecting dextran dyes into the blood stream (FIG. 4).

We found no difference in ventricle size at 28 hpf (FIG. 4A). The difference in yolk size was interpreted with caution as there might have been a time difference in fertilization between the two groups. This was not pursued further.

TABLE 6

| \check{S}ídák's | Predicted | | | | |
|---|---|---|---|---|---|
| multiple | (LS) | 95.00% | | | Adjusted |
| comparisons | mean | CI | Below | | P |
| test | diff. | of diff. | threshold? | Summary | Value |
| GPR 125 | | | | | |
| KO-AB | | | | | |
| (wildtype) | | | | | |
| 1st ventricle | −135.4 | −472.8 to | No | ns | 0.823 |
| | | 202.0 | | | |
| 2nd ventricle | 50.62 | −286.8 to | No | ns | 0.997 |
| | | 388.0 | | | |
| 3rd ventricle | −159.5 | −496.9 to | No | ns | 0.704 |
| | | 178.0 | | | |
| Eyes | 18.41 | −319.0 to | No | ns | >0.999 |
| | | 355.8 | | | |
| Yoke | 360.4 | 22.95 to | Yes | * | 0.031 |
| Diameter | | 697.8 | | | |

Regarding permeability, we observed as hypothesized, a dye size-dependent increased accumulation of dye in the KO fish. A significant higher amount of the smallest dye, 3KD, was observed in the eye, the Interpreted as a greater permeability of the blood-CSF barrier in the choroid plexus in the KO fish.

Conclusion: This example demonstrates that GPR125 is important for the permeability of compounds into the brain.

### Example 3: CP Organoids and AAV

The aim of this example was to design and validate an in vitro platform for drug screening and further investigate the role of GPR125.

### Materials and Methods:

### Animals

C57BL/6J mice were purchased from Charles River. Explants were generated from 4-6 mice per isolation. All animal experiments were approved by the Danish Animal Inspectorate (2018-15-0201-01442).

### Explant Generation

Non-fasted 8 to 10-week-old mice were euthanized by cervical dislocation, the skin on the back of the head and neck was sterilized with 70% ethanol and the brain was exposed using scissors, bone cutter, and fine forceps. The brain was excised and immersed in 10 ml of artificial CSF (aCSF) for 10 min to wash off the blood excess. The composition of aCSF was the following: 120 mM NaCl, 2.5 mM KCl, 1 mM NaH2PO4, 1.3 mM MgSO4, 17 mM Na-Hepes, 2.5 mM CaCI2 and 10 mM glucose. The pH was adjusted to 7.4 with HCl. The CP from both lateral, third and fourth ventricles was dissected under light microscope and immersed in 0.5 mL of aCSF on ice. The procedure was repeated with remaining 3-5 mice and the CP tissues were pooled in the 0.5 mL of the aCSF. When all the tissues were collected, they were minced with a pair of fine ophthalmologic scissors during 5 minutes to produce roughly 1-mm cubes. After mincing, the total volume was brought up to 1 mL by the addition of 0.5 mL aCSF. The tube contents were briefly spun and the supernatant was removed.

### Tissue Digestion

Digestion solution contained 0.25% trypsin (Life Technologies) in aCSF, and 1 mol/L EDTA (Life Technologies™), freshly prepared and kept on ice until use. 1 mL of digestion solution was added to the tube's contents and mixed by gently tapping the tube. The supernatant was aspirated using a pipette and 1-2 ml of digestion solution was added. The tube was incubated at 37° ° C. for 15-20 min with gentle pipetting with a plastic Pasteur pipette every 5 min. After the digestion, the tissue was further dispersed by gentle pipetting and checked under a microscope for presence of small fragments of CP. The digestion was stopped by adding 4 mL of DMEM containing 5% FBS to the digestion mixture and filtered through 70 μm filter. The tube was centrifuged at 300×g for 5 min at 4° C. in a 1.5 mL sterile tube. The supernatant was discarded and the pellet was washed once more with DMEM containing 5% FBS by resuspension and centrifuged again. At this point the pellet contained clusters of primary epithelial cells ranging from 20 to 50 μm. The pellet was resuspended in 200 μL of growth medium (the amount is given per pooled four CPs), containing DMEM with 5% FBS, supplemented with HEPES, L-glutamine (Sigma) and primocin 1:500 (Fisher Scientific), 1 μg/ml EGF (Peprotech), 1 μg/ml FGF10 (Peprotech), 1 μg/ml IGF1 (Peprotech), and 1:200B27 (Life Technologies). A 20 μL aliquot of cell suspension was mixed with 20 μL of 0.4% trypan blue to count cell numbers and to assess the viability. The ice-cold contents of the tube were mixed with 200 μL of Matrigel (growth factor reduced, from Corning) and 20-50 μL of the suspension were distributed in the center in each well of a 24-well glass bottomed plated (Corning). Growth medium was added to the wells after the Matrigel domes solidified. The plate was then placed in a humidified incubator with 95% air/5% CO2 at 37° C. The growth medium was used during the first 6 days of culture. Every two days, half of the medium volume was replaced with fresh growth medium. On the second day, 20 μM cytosin arabinoside (Ara-C) was added to the culture medium. After 6 days, the explants were re-plated in fresh Matrigel to eliminate cell debris and blood vessels, and to further dissociate large tissue fragments to allow explant formation. The explants were released from Matrigel by pipetting and embedded in fresh Matrigel every following 6-10 days. Ara-C treatment was applied in every passage for 48 hours to maintain fibroblast-free cultures. After the re-plating (passaging) the cultures were maintained in the growth medium, but three days before the experiments, FBS was omitted from the medium composition to promote cell maturation (Barkho & Monuki, 2015; Hakvoort et al., 1998). We maintained the cultures for at least 8 weeks.

After 30 days of culture choroid plexus organoids were transduced with either 3.3×10¹¹ GC/ml AAV5-mCherry-U6-ADRAG3-shRNA to KD GPR125 or 1.6×10¹² GC/ml AAV5-mCherry-U6-scrmb-shRNA as control. After 24 hours of AAV infection the medium was changed and cells were fixed 72 h post infection. mCherry presence was detected using a fluorescent microscope.

### Results:

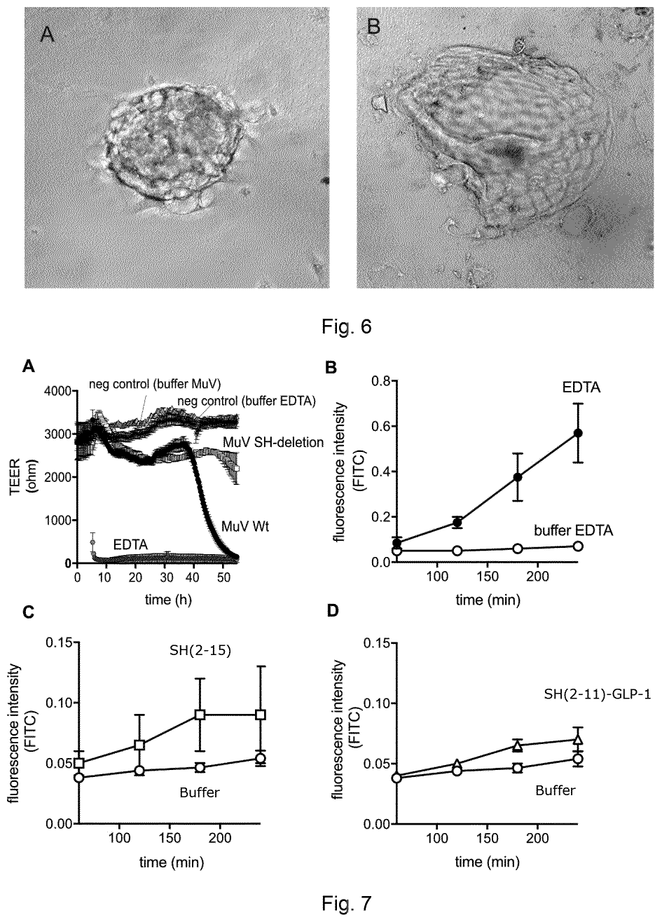

We successfully developed an in vitro platform of the choroid plexus and furthermore transduced adeno-associated virus to knock down GPR125 in the choroid plexus explants detected by mCherry fluorescence. The 3-dimensional architecture collapsed after knock down of GPR125 in the choroid plexus organoids (FIG. 6).

### Conclusion:

This example demonstrates that GPR125 is important for the 3D architecture of the CP explants.

### Example 4: TEER MuV and Membrane Permeability

The aim of this example was to study ligand induced decrease in tightness of a cell monolayer by measuring TransEpithelial Electric Resistance (TEER) using an in vitro barrier model.

Materials and methods:

CaCo-2 cells were seeded in transwell chambers (basal) and two times TEER was measured until day 17-21 post seeding. TEER experiments were performed afte 17-21 days post seeding in wells with higher TEER as 1000 ohms (proof of a tight cell monolatyer. Caco2 cells were infected basal with mumps virus (MuV Jerryl Lynn strain, WT or SH-protein deletion virus) at a multiplicity of infection of 3. TEER was measured continuously performing CellZcope experiments.

Transport studies were performed until day 17-21 post seeding as described above for the Cellzcope experiments. Ligand or control induced changes in the tightness of the cell monolayer was measured passively by the basal addition of the fluorescence dye (FITCs) together with the ligands or controls. Increase of FITCs apical was detected by collecting samples after different time points.

### Results:

TEER was reduced performing cellzcope experiments at around 37 h post infection in Caco2 cells infected with the WT MuV (FIG. 7A). This effect was not observed for Caco2 cells infected with MuV SH-protein deletion virus.

In the transport studies, induction with SH-protein(2-15) (SEQ ID NO: 92) and the SH(2-11)-GLP-1(7-36) (SEQ ID NO: 128) resulted in decreased integrity of tight monolayer of CaCO-2 cells, as observed by an increased uptake of fluorescent dye FITCs (FIG. 7C and D).

### Conclusion:

This example demonstrates that the SH-protein interferes with the integrity of the tight monolayer of CaCo-2 cells, resulting in increased uptake of fluorescent dye FITCs. The example further demonstrates that a fragment of SH-protein (SH(2-15) (SEQ ID NO: 92)) is capable of inducing the effect and that conjugation of a drug to the SH-protein SH(2-11) does not prevent the SH-protein from inducing the effect.

### Example 5: TAMRA Labelled SH-Protein Binding to Mouse Choroid Plexus

Aim: The aim of this example was to determine the binding of the SH-protein in epithelia with high GPR125 expression compared to epithelia with reduced GPR125 expression.

### Materials and Methods:

### Animals

C57BL/6J mice were purchased from Charles River. Explants were generated from 4-6 mice per isolation. All animal experiments were approved by the Danish Animal Inspectorate (2018-15-0201-01442).

SH protein (SEQ ID NO: 74) with a N-terminal carboxytetramethyl rhodamine attached through its carboxyl-group to the N-terminal alpha amino-group of the first Methionine was used for making the TAMRA labelled SH-protein (SEQ ID NO: 129). A mixture of 5-(and-6)-Carboxytetramethylrhodamine was used.

A whole body perfusion was performed of mice and brains were dissected and frozen at −80C. Cryo-sections of mouse brains (WT and GPR125 KO mouse (Spina et al. 2021)) were generated and the TAMRA-labelled SH-protein was incubated on the cryosections at different concentrations or buffer. Afterwards the sections were mounted with mounting media containing a DAPI stain.

### Results:

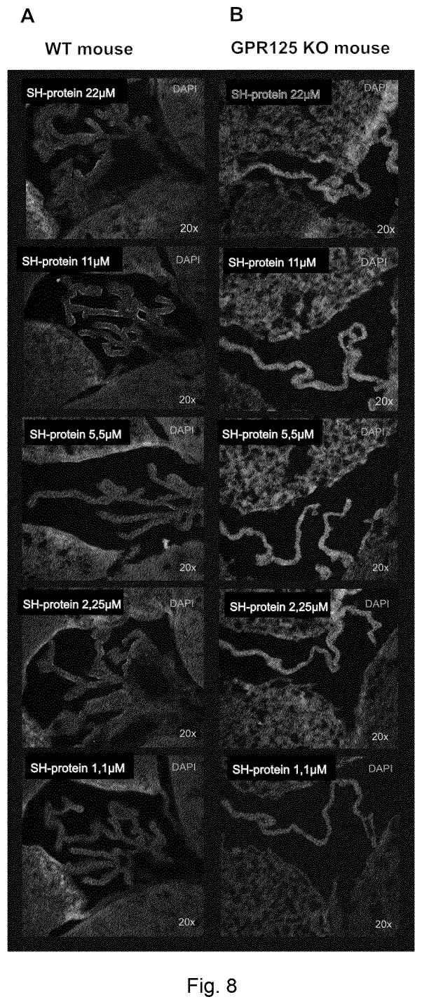

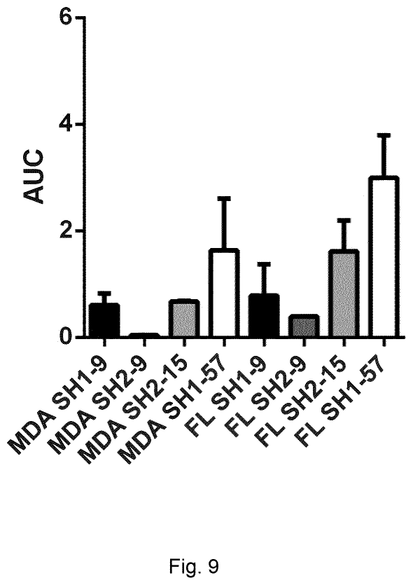

The TAMRA labelled SH-protein binds to the choroid plexus of the WT mouse brain still at a concentration of 2,25 UM (FIG. 8, visible as fine white line around the choroid plexus). The TAMRA labelled SH-protein binds to the choroid plexus of the GPR125 KO mouse brain only at higher concentrations of 22 μM and 11 μM (visible as a fine white line around the choroid plexus).

### Conclusion:

This example demonstrates that the TAMRA labelled SH-protein binds to the choroid plexus of the mouse choroid plexus expressing GPR125 in a dose dependent manner and only at higher concentrations to the GPR125 KO mouse choroid plexus.

### Example 6: xCELLigence Stimulation with SH Protein Fragments

Aim: The aim of this example was to determine whether N-terminal peptides of the SH protein affected label-free signaling of GPR125.

Materials and methods:

The specific E-plates used with this system are coated with gold electrodes and the cells are seeded directly on top of these, hence the more a cell attaches or spreads out on the electrode the larger the impedance measurement will be when a current of 10 kHz is run through the plate. Once the cells are added to the E-plate they are allowed to adhere and proliferate for 18 hours during which the impedance is measured every 15 minutes generating the initial adhesion and growth curve. Upon stimulation the measurements are change to rapid detection every 15 seconds followed by every minute and every 5 minutes during the first hour after stimulation.

### Result:

Determined by the area under the curve (AUC), SH protein fragments of varying size; SH1-9 (SEQ ID NO: 91), SH2-9 (SEQ ID NO: 98), SH2-15 (SEQ ID NO: 92), and whole protein SH1-57 (SEQ ID NO: 115) affected GPR125 signaling either for the receptor expressed endogenously (MDA231 cells) or in HEK293 Flp-In T-rex cells stably transfected with full length (FL) GPR125.

### Conclusion:

This example demonstrates an impact of mumps virus SH-protein on GPR125 signaling, hence supporting a direct interaction of the SH-protein and fragments of SH-protein with GPR125.

### Example 7: Peptide Synthesis

Peptides were synthesized by solid-phase peptide synthesis using a Biotage Initiator+Alistar™ Auto-mated Microwave Peptide Synthesizer utilizing the standard Fmoc/tBu strategy [Curr Protoc Protein Sci. 2002, CHAPTER: Unit-18.1]. As solid polymeric support a ChemMatrix resin (100% PEG, loading ˜ 0.5 mmol/g) was used, which was functionalized with a Rink amide linker to release the C-terminus as an amide when the peptide was cleaved from the solid support at the end of the synthesis. The α-amines were Fmoc protected and unless otherwise mentioned side-chains were protected (when necessary) by standard acid-labile protecting groups (tBu, Boc, Trityl (Trt) and 2,2,4,6,7-Pentamethyl-dihydrobenzofuran-5-sulfonyl (Pbf)).

### Synthesis of SH(2-11)-GLP-1(7-36) Conjugate

GLP-1(7-36) (SEQ ID NO: 127) was synthesized according to ‘Peptide Synthesis’. Lys26 was installed with the sidechain amino group Alloc-protected, allowing selectively removal of this protecting group. The N-terminal His7 was installed with the α-amino group Boc-protected.

After synthesis of GLP-1(7-36), the Lys(Alloc) group was selectively deprotected: The resin was washed with DMF (×5), MeOH (×6) and CH₂Cl₂ (×6) and dried in a vacuum desiccator overnight. Next a solution of Pd(PPh₃)₄ (0.5 equiv) and phenylsilane (24 equiv) in dry CH₂Cl₂ was added to the resin in a dry round bottom flask. The mixture was shaken for 2 h under nitrogen. The resin was then drained and left on high vacuum for 2 hours and new amounts of the reagents were added, and the reaction was repeated for another 2 hours. Then the resin was drained and washed with CH₂Cl₂, DMF and CH₂Cl₂ again. The resin was washed with CH₂Cl₂ (×5), 0.5% (v/v) DIPEA-DMF (×3), 0.5% (w/v) sodium diethylthiocarbamate-DMF (×5), CH₂Cl₂ (×5) and DMF (×5) before transferred to the Biotage Initiator+Alistar™ Automated Microwave Peptide Synthesizer.

Synthesis proceeded according to ‘Peptide Synthesis’. The first coupling was done with 1-(9H-fluoren-9-yl)-3-oxo-2,7, 10-trioxa-4-azadodecan-12-oic acid, followed by the SH(2-11) sequence, hereby generating the SH(2-11)-GLP-1(7-36) conjugate (SEQ ID NO: 128 and FIG. 12). Installation of the polyethylene glycol-2 (PEG2)-spacer was found to be crucial for solubility and we were never able to isolate the product by preparative HPLC without a spacer due to low aqueous solubility. Although the product could be identified by MALDI-MS, no further efforts to isolate the product was attempted, as the low aqueous solubility rendered the peptide not relevant for biological applications.

The product was released from resin according to ‘Release of peptide from resin+Cleavage of side chain protecting groups’ and analyzed according to ‘Analysis of peptide products’.

### Synthesis of SH(2-11)-Exendin-4(1-39) Conjugate

Exendin-4(1-39) (SEQ ID NO: 130) was synthesized according to ‘Peptide Synthesis’. Lys27 was installed with the sidechain amino group Alloc-protected, allowing selectively removal of this protecting group. The N-terminal His1 was installed with the α-amino group Boc-protected.

After synthesis of Exendin-4(1-39), the Lys(Alloc) group was selectively deprotected: The resin was washed with DMF (×5), MeOH (×6) and CH₂Cl₂ (×6) and dried in a vacuum desiccator overnight. Next a solution of Pd(PPh₃)₄ (0.5 equiv) and phenylsilane (24 equiv) in dry CH₂Cl₂ was added to the resin in a dry round bottom flask. The mixture was shaken for 2 hours under nitrogen. The resin was then drained and left on high vacuum for 2 hours and new amounts of the reagents were added, and the reaction was repeated for another 2 hours. Then the resin was drained and washed with CH₂Cl₂, DMF and CH₂Cl₂ again. The resin was washed with CH₂Cl₂ (×5), 0.5% (v/v) DIPEA-DMF (×3), 0.5% (w/v) sodium diethylthiocarbamate-DMF (×5), CH₂Cl₂ (×5) and DMF (×5) before transferred to the Biotage Initiator+Alistar™ Automated Microwave Peptide Synthesizer.

Synthesis proceeded according to ‘Peptide Synthesis’. The first coupling was done with 1-(9H-fluoren-9-yl)-3-oxo-2,7, 10-trioxa-4-azadodecan-12-oic acid, followed by the SH(2-11) sequence, hereby generating the SH(2-11)-Exendin-4(1-39) conjugate (SEQ ID NO: 131 and FIG. 13). Installation of the polyethylene glycol-2 (PEG2)-spacer was found to be crucial for solubility.

The product was released from resin according to ‘Release of peptide from resin+Cleavage of side chain protecting groups’ and analyzed according to ‘Analysis of peptide products’.

Release of Peptide from Resin+Cleavage of Side Chain Protecting Groups

The functionalized resin was swelled in a cleavage cocktail containing TFA: H₂O:triisopropylsilane 95:2.5:2.5:5 (1 mL/30 mg coupled resin), shaking for 24 h hours), before filtration and concentration of the cleavage cocktail to around 2 mL. Addition of diethylether resulted in precipitation of the product peptide as a white fluffy powder. Peptide was isolated by centrifugation and solvent removed by decanting. Product was washed with diethylether and deptide isolated by the above-described centrifugation and decanting procedure. This was repeated 5 times.

Centrifugation of the precipitated crude peptide was performed using a SIGMA 2-6E Centrifuge.

Preparative HPLC purification of the peptides was performed on a Waters autopurification system consisting of a 2767 Sample Manager, 2545 Gradient Pump and 2998 PDA detector. Column: XBridge Peptide BEH C18 OBD Prep Column, 130 Å, 5 μm, 19 mm×100 mm. Column temp: Ambient. Flow rate: 20 mL/min. Solvent A2—15 mM NH₄Ac in water, Solvent B2—15 mM NH₄Ac in MeCN/water 9:1. Gradient: 5% B in 3 min., gradient: 5% B to 20% B in 2 min., gradient: 20% B to 50% B in 2 min., hold 2 min., gradient: 50% B to 70% B in 3 min., gradient: 70% B to 100% B in 3 min., hold 0.5 min., run 15.5 min., recalibrating the column for 2.5 min. Total run time—18 min.

Lyophilization of the purified peptides was performed using a ScanVac CoolSafe freeze dryer after freezing the samples on dry ice.

### Analysis of Peptide Products

UPLC-MS analysis was run on Waters AQUITY UPLC system equipped with PDA and a SQD2 (for the peptides) electrospray MS detector. Column: Thermo accucore C18 2.6 m, 2.1 50 mm. Column temp: 50° C. Flow rate: 0.6 mL/min. Acid run: Solvent A1 -0.1% formic acid in water, Solvent B1—0.1% formic acid in MeCN. Base run: Solvent A2—15 mM NH₄Ac in water, Solvent B2—15 mM NH₄Ac in MeCN/water 9:1. For determining the mass of the peptides 5 min. base runs were used. Gradient: 5% B to 100% B in 3 min., hold 0.1 min. Total run time—5 min.

UPLC-MS analysis confirmed the identity of SH(2-11)-GLP-1(7-36) [M, C₂₀₉H₃₂₀N₅₄O₆₁] as observed masses: mz=1521.7 [M+3H]⁺³, 1542: [M+4H₂O]⁺².

UPLC-MS analysis confirmed the identity of SH(2-11)-Exendin-4(1-39) [M, C₂₄₄H₃₇₆ N₆₂O₇₆S] as observed masses: m/z=1810.8 [M+3H]⁺³, 1427: [M+16H₂O]⁺⁴.

HPLC/ELSD analysis was run on an e2695 Waters Alliance system equipped with a 2998 PDA detector and an Agilent Technologies 1260 Infinity ELSD. Column: Symmetr C18 3.5 m, 4.6 mm 75 mm. Column temp: 20° C. Flow rate: 1 mL/min. Solvent A2—15 mM NH₄Ac in water, Solvent B2—15 mM NH₄Ac in MeCN/water 9:1. Gradient: 5% B in 0.5 min., gradient: 5% B to 70% B in 9.5 min., hold 2 min., gradient: 70% B to 100% B in 5 min., hold 3 min., run 20 min., recalibrating the column for 2 min. Total run time—22 min. HPLC was used to check the purity of the peptides.

MALDI/MS analysis was run on a Bruker AutoFlex Speed MALDI TOF mass spectrometer set in a linear, positive mode. The benchtop instrument was equipped with a BRUKER smartbeam™-II laser (Amax=355 nm) and pulse extraction of 23,000 nanoseconds was performed. A 2,5-Dihydroxybenzoic acid (DHB) solution was used as matrix for analysis. A total of 4000 shots in 200 positions of a single plate well were obtained.

MALDI-TOF/MS analysis confirmed the identity of SH(2-11)-GLP-1(7-36) [M, C₂₀₉H₃₂₀N₅₄O₆₁] as an observed mass: m/z=4566.851 [M+H]⁺.

MALDI-TOF/MS analysis confirmed the identity of SH(2-11)-Exendin-4(1-39) [M, C₂₄₄H₃₇₆ N₆₂O₇₆S] as an observed mass: m/z=5424.7 [M+H]⁺.

### Example 8: Food Intake and GLP-1 Activity

Aim: The aim of this example was to investigate changes of the centrally acting anorectic effect of GLP-1(7-36) upon fusion with the SH-protein SH(2-11) in live animals

### Materials and Methods:

### Animals

Female C57BL/6J mice were purchased from Charles Rivers. Animals were fasted for 10 hours during light-phase, and just as dark-phase started (6 PM), animals were injected with either vehicle (5% DMSO, 10% Tween80, 85% milliQ water, n=4), 900 nmol/kg GLP-1(7-36) (SEQ ID NO: 127) (n=2), 200 nmol/kg GLP-1(7-36) (n=4) (SEQ ID NO: 127), or 200 nmol/kg SH(2-11)-GLP-1(7-36) (SEQ ID NO: 128) (n=5) by i.p. injections. The animals were housed in individual cages, with access to food and water, and food intake was measured 1 and 2 hours after injection.

cAMP Accumulation Assay

COS-7 cells were cultured at 10% CO₂ and 37° C. in Dulbecco's modified Eagles medium 1885 supplemented with 10% FBS, 2 mM glutamine, 180 units/ml penicillin, and 45 g/ml streptomycin. Transient transfection of COS-7 cells was performed using calcium phosphate precipitation method. Briefly, a total of 10 ug of receptor DNA was diluted in TE-buffer (10 nM Tris-HCl and 1 mM EDTA, pH 7.5) to a total of 105 ul, to which 15 ul of 2 M CaCl₂) was added. The DNA-calcium co-precipitation was gently and slowly added to 120 ul 2× HEPES-buffered saline (HBS), and the transfection mixture was incubated at room temperature for 45 min. The total transfection mixture was added to the cells and incubated for 5 hours at 37° C. and 10% CO₂, with the addition of 75 ul chloroquine to the growth medium. After 5 hours, the transfection medium was removed and replaced with regular growth medium. The cells were used in experiment 40-48 h after termination of the transfection procedure.

For the cAMP assay, the COS-7 cells were seeded into 96-well plates (35.000 cells/well) and washed with HBS prior to incubation with 100 ul 250 mM isobutylmethylzanthine (IBMX) for 30 min at 37° C. Ligands was added (GLP-1 or GLP-1_SH) and incubated for further 30 min at 37° C. After incubation, the medium was removed, and cells were treated according to the protocol ‘three reagent addition’ procedure for the HitHunter cAMP XS+Assay by DiscoverX, USA. The amount of cAMP was measured as luminescence using the PerkinElmer EnVision 2104 Multilable Reader.

### Results:

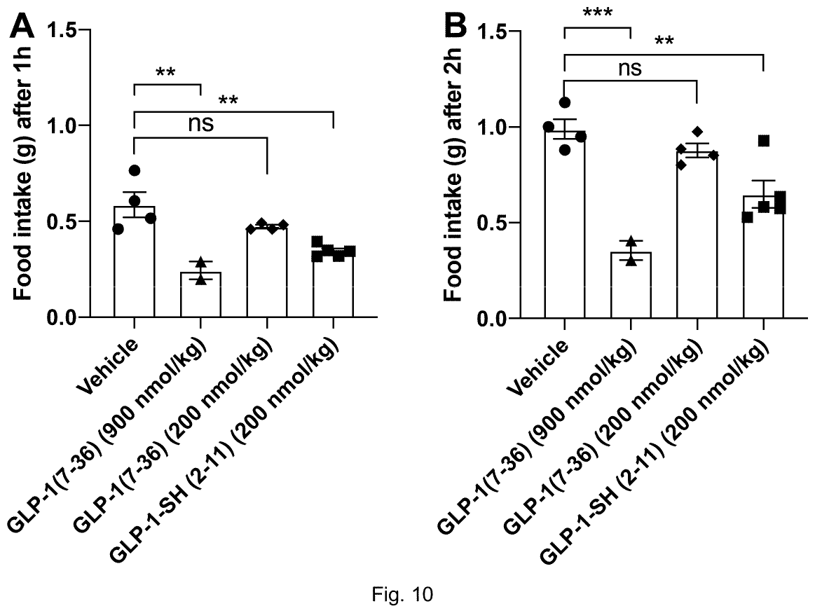

Food intake data show that the SH(2-11)-GLP-1(7-36) fusion protein (200 nmol/kg) decreases the food intake significantly, compared to vehicle injected animals, after both 1 (FIG. 10A) and 2 hours (FIG. 10B), and even to a greater extent than the control group injected with 200 nmol/kg GLP-1.

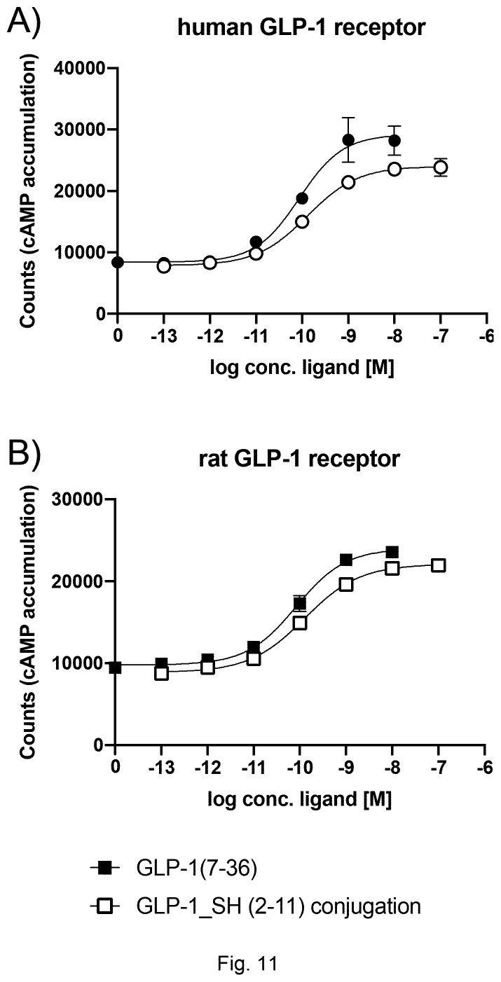

The function of the GLP-1 molecule on binding and activating the human (FIG. 11A) and rat GLP-1R (FIG. 11B) by cAMP accumulation, is unchanged by the fusion with the SH-protein fragment.

Conclusion: This example demonstrates that the conjugation of GLP-1(7-36) to the SH protein fragment SH(2-11) does not affect the ability of GLP-1 to bind and activate the human and rat GLP-1R both in vitro and in vivo. Furthermore, it demonstrates that the central anorectic effect of SH(2-11)-GLP-1(7-36) fusion protein is higher than unmodified GLP-1(7-36), indicating that a higher amount of the SH(2-11)-GLP-1(7-36) fusion protein enters the brain as compared to unmodified GLP-1(7-36).

### Example 9: CSF Concentration

Aim: The aim of this example was to investigate the uptake of SH(2-11)-GLP-1(7-36) fusion protein (SEQ ID NO: 128) into the brain in live animals in comparison with uptake of unmodified GLP-1(7-36) (SEQ ID NO: 127).

### Materials and Methods:

### Animals

Male Sprague Dawley rats were purchased from Charles Rivers. Animals were anesthetized by hypnorm/dormicum, and restrained in a stereotactic instrument. Injections directly into vena cava were done with either vehicle (5% DMSO, 10% Tween80, 85% milliQ water, n=1), 50 nmol/kg GLP-1 (SEQ ID NO: 127) (n=1) or 500 nmol/kg SH(2-11)-GLP-1(7-36) (SEQ ID NO: 128) (n=1) with an injection volume of 500 ul/rat. 10 minutes after, cerebrospinal fluid (CSF) was aspirated, by exposing the scull and neck muscles and a puncture of cisterna magna with a small glass capillary. Blood samples were collected prior to the injection of test compounds and straight after CSF aspiration.

### Measurements of Plasma and CSF Concentrations (RIA GLP-1 Assay)

GLP-1(7-36) and SH(2-11)-GLP-1(7-36) concentrations in plasma and CSF were measured by a method adapted from Deacon et al. 2002. In brief, for GLP-1 measurements, GLP-1(7-36) was used as standard and for SH(2-11)-GLP-1(7-36) measurements, SH(2-11)-GLP-1(7-36) was used as standard. The assay buffer was 100 mM Tris buffer with pH 8.5 containing 1% (wt/vol) human serum albumin, 0.01 mM valine-pyrrolidide and 500 KIE aprotinin (final concentrations). The antibody (code no. 98302) was diluted to a final titer of 1:120000 and the tracer was 125-I-labeled GLP-1. Plasma and CSF concentrations were measured after dilution in assay buffer. Free and bound moieties were separated with plasma-coated charcoal.

### Results:

The data confirm that GLP-1(7-36) and SH(2-11)-GLP-1(7-36) both are measurable by RIA GLP-1 assay, and that both are detectable in CSF 10 min after i.v. injection. The amount of detectable SH(2-11)-GLP-1(7-36) within the CSF were approximately 55% of the total injected concentration, while GLP-1(7-36) present within the CSF were approximately 6% of the total injected concentration (Table 7), meaning that the SH(2-11)-GLP-1(7-36) fusion protein is concentrated 10-times more in the CSF compared to GLP-1(7-36).

TABLE 7

| | Injection conc [μM] | CSF conc [μM] | Ratio |
|---|---|---|---|
| Vehicle | 0 | ND | — |
| SH(2-11)-GLP-1 | 500 | 277.0 | 0.554 |
| GLP-1 | 50 | 2.9 | 0.058 |

### Conclusion:

This example demonstrates that the SH(2-11)-GLP-1(7-36) fusion protein is detectable by a GLP-1 RIA assay, both in plasma and CSF. Furthermore, the fusion protein has a 10-fold higher presence within the CSF, indicating an increased entrance across the blood-brain barrier, when GLP-1(7-36) is conjugated to the SH(2-11) protein.

### Example 10: Food Intake and SH(2-11)-GLP-1(7-36) Activity in Lean Mice

Aim: The aim of this example was to investigate changes of the centrally acting anorectic effect of GLP-1(7-36) upon fusion with the SH-protein SH(2-11) in mice, at different dose concentrations.

### Materials and Methods:

### Animals

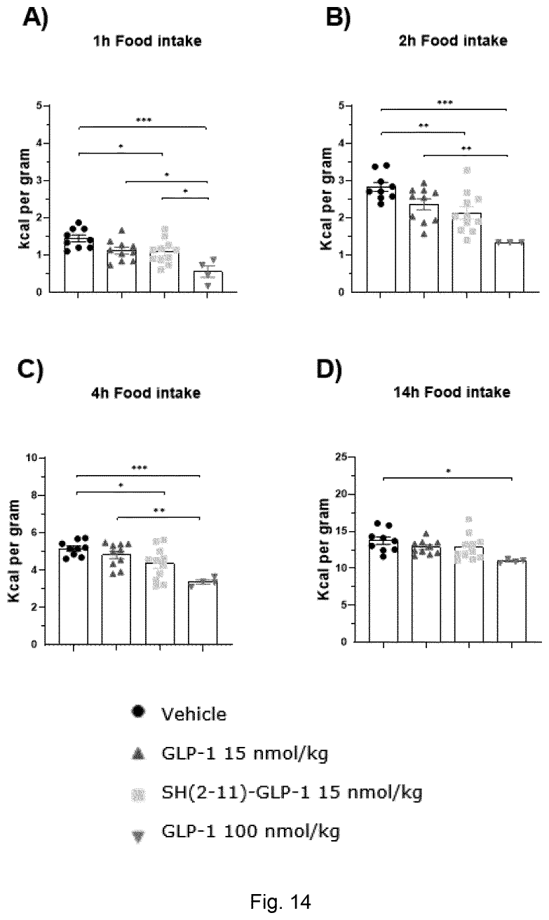

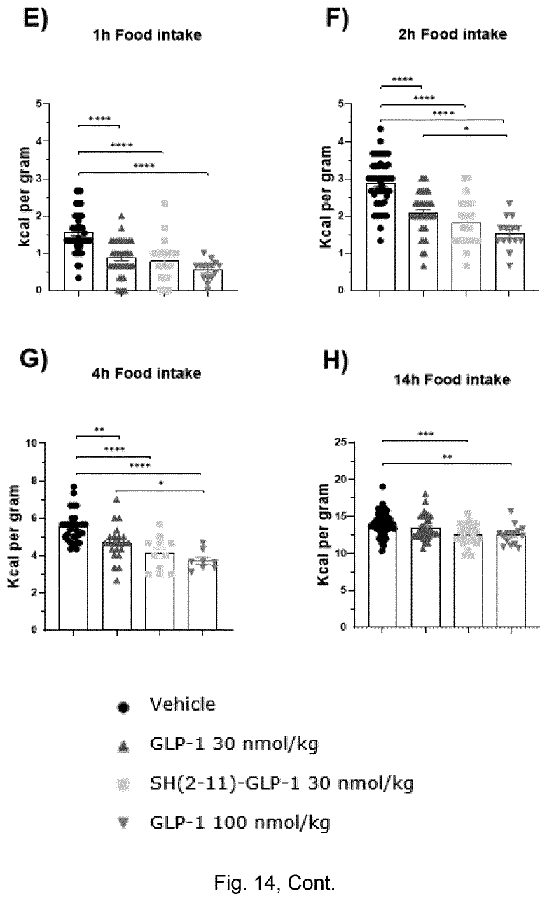

Female C57BL/6J mice were purchased from Charles Rivers. One week before study initiation the mice were single housed with free access to food and water. On the experimental day animals were fasted for 6 hours during light-phase, and right before dark phase (6PM) mice were injected subcutaneously with either vehicle (0,05 UM acetic acid in 0,9% saline), n=9), 15 nmol/kg GLP-1(7-36) (SEQ ID NO: 127) (n=10), 15 nmol/kg SH(2-11)-GLP-1(7-36) (SEQ ID NO: 128) (n=11) or 100 nmol/kg GLP-1(7-36) (n=3-4) (used as positive controls). Thirty minutes prior peptide injection, all mice were administered subcutaneously with DDP-4 inhibitor Valine-pyrrolidide (1.5 mg/mouse). Food intake was measured after 1, 2, 4 and 14 hours (FIG. 14A-D).

The experiment was repeated with mice receiving either vehicle (0,05 UM acetic acid in 0,9% saline), n=50), 30 nmol/kg GLP-1(7-36) (n=36), 30 nmol/kg SH(2-11)-GLP-1(7-36) (n=24) or 100 nmol/kg GLP-1(7-36) (n=15) (FIG. 14E-H). Results shown are combined data from five studies with an identical study design as described above. Between each study mice had at least one week washout period.

### Results:

15 nmol/kg: Mice injected with SH(2-11)-GLP-1(7-36) fusion protein (15 nmol/kg) decreased food intake significantly, compared to vehicle injected animals after 1, 2 and 4 hours. Native GLP-1(7-36) at 15 nmol/kg did not show a significant decrease in food intake compared to the vehicle treated mice (FIG. 14A-D).

30nmol/kg: Mice treated with GLP-1(7-36) and SH(2-11)-GLP-1(7-36) at 30nmol/kg decreased their food intake significantly after 1 and 2 hours compared to vehicle. This effect was still present after 4 hours but GLP-1(7-36) mice increased their food intake at this time point compared to SH(2-11)-GLP-1(7-36) and the effect was less significant compared to vehicle. At 14 hours the mice injected with SH(2-11)-GLP-1(7-36) still had a significant lower food intake vs. vehicle (FIGS. 14E-H).

Conclusion: This example demonstrates that the conjugation of GLP-1(7-36) to the SH protein fragment SH(2-11) does not affect the ability of GLP-1(7-36) to activate the GLP-1R in vivo. Furthermore, it demonstrates that the central anorectic effect of SH(2-11)-GLP-1(7-36) fusion protein is higher than unmodified GLP-1(7-36), indicating that a higher amount of the SH(2-11)-GLP-1(7-36) fusion protein enters the brain as compared to unmodified GLP-1(7-36).

### Example 11: Food Intake and SH(2-11)-GLP-1(7-36)Activity in Diet-Induced Obese Mice

Aim: The aim of this example was to investigate changes of the centrally acting anorectic effect of GLP-1(7-36) upon fusion with the SH-protein SH(2-11) in mice fed a high fat high sucrose diet.

### Materials and Methods:

### Animals

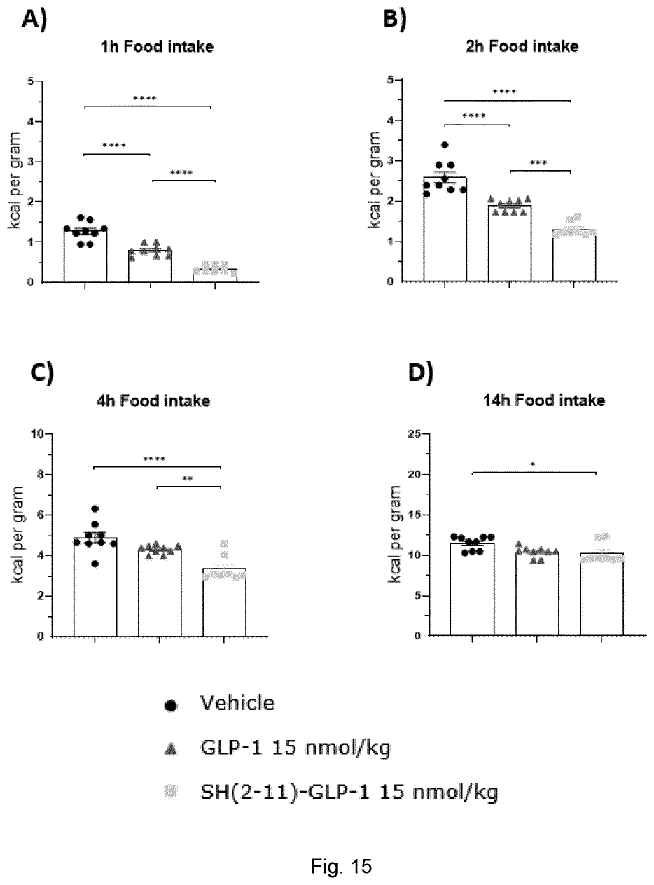

Diet induced obese C57BL/6NTac male mice were purchased from Taconic and continued on a high fat high sucrose diet with free access to water. One week after arrival, animals were double or single housed. On the experimental day animals were fasted for 4 hours during light-phase, and right before dark phase (6PM) mice were injected subcutaneously with either vehicle (0,05 UM acetic acid in 0,9% saline), n=9), 15 nmol/kg GLP-1(7-36) (SEQ ID NO: 127) (n=9), 15 nmol/kg or SH(2-11)-GLP-1(7-36) (SEQ ID NO: 128) (n=8). Thirty minutes prior peptide injection, all mice were administered subcutaneously with DDP-4 inhibitor Valine-pyrrolidide (3 mg/mouse). Food intake was measured after 1, 2, 4 and 14 hours (FIG. 15A-D).

### Results:

SH(2-11)-GLP-1(7-36) fusion protein decreased food intake significantly, compared to both vehicle and GLP-1(7-36) after 1, 2 and 4 hours. GLP-1(7-36) also decreased food intake after 1 and 2 hours compared to vehicle but at 4 hours this effect was not present. Food intake at 14 hours was still significantly decreased with SH(2-11)-GLP-1(7-36) mice vs. vehicle (FIG. 15A-D).

Conclusion: This example demonstrates that the central anorectic effect of SH(2-11)-GLP-1(7-36) fusion protein is higher than unmodified GLP-1(7-36), indicating that a higher amount of the SH(2-11)-GLP-1(7-36) fusion protein enters the brain as compared to unmodified GLP-1(7-36). The effect on food intake was more evident in diet induced obese mice compared to lean mice treated with the same dose.

### Example 12: Food Intake and SH(2-11)-Exendin-4 Activity In Vivo

Aim: The aim of this example was to investigate changes of the centrally acting anorectic effect of Exendin-4(1-39) upon fusion with the SH-protein SH(2-11) in mice.

### Materials and Methods:

### Animals

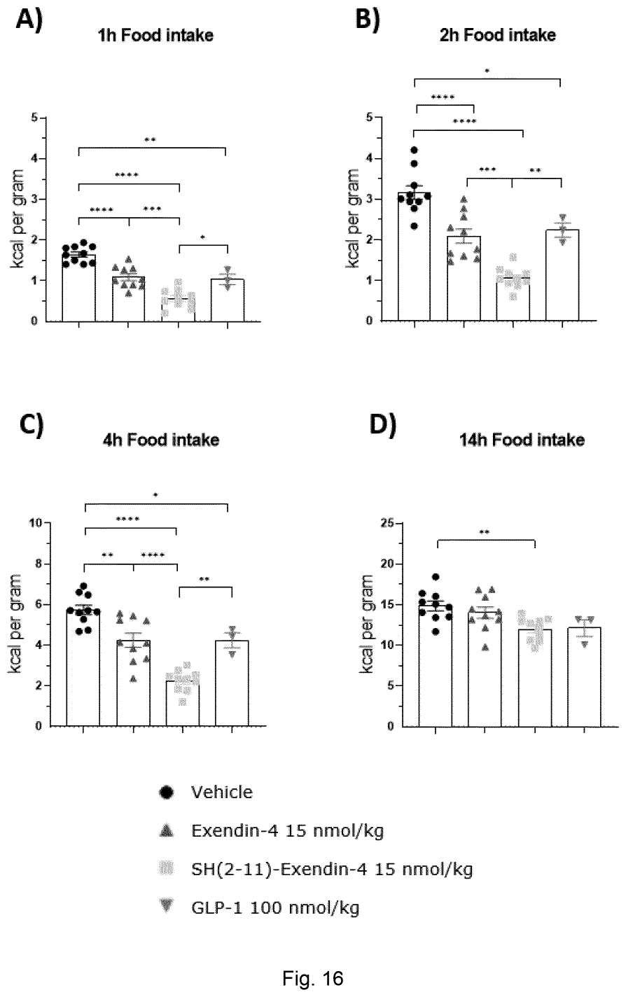

Female C57BL/6J mice were purchased from Charles Rivers. One week before study initiation the mice were single housed with free access to food and water. On the experimental day animals were fasted for 6 hours during light-phase, and right before dark phase (6PM) mice were injected subcutaneously with either vehicle (0,05 UM acetic acid in 0,9% saline), n=10), 15 nmol/kg Exendin-4(1-39) (SEQ ID NO: 130) (n=10), 15 nmol/kg SH(2-11)-Exendin-4(1-39) (SEQ ID NO: 131) (n=10) or 100 nmol/kg GLP-1(7-36) (SEQ ID NO: 127) (n=3) used as positive controls. Food intake was measured after 1, 2, 4 and 14 hours (FIG. 16A-D).

### Results:

Food intake at 1, 2 and 4 hours was significantly decreased in mice treated with SH(2-11)-Exendin-4(1-39) fusion protein compared to both vehicle and Exendin-4(1-39). Exendin-4(1-39) also decreased food intake after 1 and 2 hours compared to vehicle but at 4 hours this effect was less significant. Food intake at 14 hours was still significantly decreased with SH(2-11)-Exendin-4(1-39) mice vs. vehicle (FIG. 16A-D).

Conclusion: This example demonstrates that the central anorectic effect of SH(2-11)-Exendin-4(1-39) fusion protein is higher than unmodified Exendin-4(1-39), indicating that a higher amount of the SH(2-11)-Exendin-4(1-39) fusion protein enters the brain as compared to unmodified Ecxendin-4(1-39).

1. A compound comprising a structure according to formula (I) 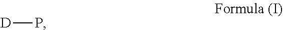
wherein
D comprises or consists of a drug; and;
P comprises or consists of a mumps virus small hydrophobic protein (MuV SH protein) or a variant, a fragment, or a variant of a fragment thereof,
wherein D is conjugated to P, optionally via a linker L.

2. The compound according to claim 1, wherein P originates from a clinical isolate or a patient isolate of mumps virus.

3. The compound according to any one of the preceding claims, wherein P originates from a genotype G mumps virus.

4. The compound according to any one of the preceding claims, wherein P originates from a MuV vaccine strain, such as originates from a Jeryl-Lynn strain, Urabe AM9 strain, Leningrad 3 strain, RIT 4385 strain, Leningrad-Zagreb strain, S79 strain, Rubini strain, Hishino strain, RS(S-12) strain, Torii strain, or Miyahana strain.

5. The compound according to any one of the preceding claims, wherein P originates from a genotype G or vaccine strain mumps.

6. The compound according to any one of the preceding claims, wherein P comprises or consists of an amino acid sequence of MPAIX₁PPX₂X₃X₄ TFLLLX₅LLX₆L IX₇TLYVWX₈X₉X₁₀ X₁₁X₁₂X₁₃X₁₄X₁₅TX₁₆ VRX₁₇ AX₁₈LX₁₉QRSX₂₀X₂₁X₂₂ WX₂₃X₂₄DX₂₅X₂₆L (SEQ ID NO: 1); wherein:
X₁ is Q or R
X₂ is L or S
X₃ is Y or H
X₄ is L or P
X₅ is I or T
X₆ is Y or S
X₇ is I, T or V
X₈ is I or T
X₉ is I or T
X₁₀ is L or S
X₁₁ is T or A
X₁₂ is V or I
X₁₃ is T or N
X₁₄ is Y or H
X₁₅ is K or N
X₁₆ is A or V
X₁₇ is H, P, Y or R
X₁₈ is A, T, or S
X₁₉ is Y or H
X₂₀ is F, C or S
X₂₁ is F, V or S
X₂₂ is H or R
X₂₃ is S, R or G
X₂₄ is F or L
X₂₅ is H or Q
X₂₆ is S, P, or T,
or a fragment thereof, a variant thereof, or a variant of a fragment thereof.

7. The compound according to claim 6, wherein said fragment of P comprises at least amino acids 2-8 (PAIX₁PPX₂), such as comprises at least amino acids 2-9 (PAIX₁PPX₂X₃), and
wherein said variant of P comprises one or more amino acid substitutions, such as one amino acid substitution, such as two amino acid substitutions, such as three amino acid substitutions, such as four amino acid substitutions, such as five amino acid substitutions, and
wherein said variant of said fragment of P comprises at least amino acids 2-8 (PAIX₁PPX₂), such as comprises at least amino acids 2-9 (PAIX₁PPX₂X₃), and having one or more amino acid substitutions, such as one amino acid substitution, such as two amino acid substitutions, such as three amino acid substitutions, such as four amino acid substitutions, such as five amino acid substitutions within said fragment.

8. The compound according to any one of the preceding claims, wherein P originates from a genotype G mumps virus.

9. The compound according to any one of the preceding claims, wherein P comprises or consists of an amino acid sequence of PAIX₁PPX₂YLTFLLLILLYLIX₃TLYVWIILX₄VTYKTAVRX₅AALYQRSX₆X₇HWX₈F DHX₉L (SEQ ID NO: 2); wherein:
X₁ is Q or R;
X₂ is L or S;
X₃ is I or T;
X₄ is T or A;
X₅ is H, P or R;
X₆ is F or S;
X₇ is F or V;
X₈ is Sor R; and
X₉ is S or T,
or a fragment thereof, a variant thereof, or a variant of a fragment thereof.

10. The compound according to claim 9, wherein said fragment of P comprises at least amino acids 1-8 (PAIX₁PPX₂Y), such as comprises at least amino acids 1-9 (PAIX₁PPX₂YL), and
wherein said variant of P comprises one or more amino acid substitutions, such as one amino acid substitution, such as two amino acid substitutions, such as three amino acid substitutions, such as four amino acid substitutions, such as five amino acid substitutions, and
wherein said variant of said fragment of P comprises at least amino acids 1-8 (PAIX₁PPX₂Y), such as comprises at least amino acids 1-9 (PAIX₁PPX₂YL), and having one or more amino acid substitutions, such as one amino acid substitution, such as two amino acid substitutions, such as three amino acid substitutions, such as four amino acid substitutions, such as five amino acid substitutions within said fragment.

11. The compound according to any one of the preceding claims, wherein P originates from a Vaccine strain mumps virus.

12. The compound according to any one of the preceding claims, wherein P comprises or consists of an amino acid sequence of PAIQPPLX₁X₂ TFLLLX₃LLX₄L IX₅TLYVWX₆X₇X₈ TIX₉X₁₀X₁₁TX₁₂VRX₁₃ AX₁₄LX₁₅QRSX₁₆X₁₇R WX₁₈X₁₉DX₂₀X₂₁L (SEQ ID NO: 3); wherein:
X₁ is Y or H
X₂ is L or P
X₃ is I or T
X₄ is Y or S
X₅ is I or V
X₆ is I or T
X₇ is I or T
X₈ is L or S
Xg is T or N
X₁₀ is Y or H
X₁₁ is K or N
X₁₂ is A or V
X₁₃ is Y or H
X₁₄ is A, T, or S
X₁₅ is H or Y
X₁₆ is F or C
X₁₇ is F or S
X₁₈ is S or G
X₁₉ is F or L
X₂₀ is H or Q, and
X₂₁ is S or P,
or a fragment thereof, a variant thereof, or a variant of a fragment thereof.

13. The compound according to claim 12, wherein said fragment of P comprises at least amino acids 1-8 (PAIQPPLX₁), such as comprises at least amino acids 1-9 (PAIQPPLX₁X₂), and
wherein said variant of P comprises one or more amino acid substitutions, such as one amino acid substitution, such as two amino acid substitutions, such as three amino acid substitutions, such as four amino acid substitutions, such as five amino acid substitutions, and
wherein said variant of said fragment of P comprises at least amino acids 1-8 (PAIQPPLX₁), such as comprises at least amino acids 1-9 (PAIQPPLX₁X₂), and having one or more amino acid substitutions, such as one amino acid substitution, such as two amino acid substitutions, such as three amino acid substitutions, such as four amino acid substitutions, such as five amino acid substitutions within said fragment.

14. The compound according to any one of the preceding claims, wherein P comprises or consists of an amino acid sequence of PAIQPPLYLT FLLLILLYLI ITLYVWIILT VTYKTAVRHA ALYQRSFFHW SFDHSL (SEQ ID NO: 63), or a fragment thereof, a variant thereof, or a variant of a fragment thereof.

15. The compound according to claim 14, wherein said fragment of P comprises at least amino acids 1-8 (PAIQPPLY), such as comprises at least amino acids 2-9 (PAIQPPLYL), and
wherein said variant comprises one or more amino acid substitutions, such as one amino acid substitution, such as two amino acid substitutions, such as three amino acid substitutions, such as four amino acid substitutions, such as five amino acid substitutions, and
wherein said variant of said fragment comprises at least amino acids 1-8 (PAIQPPLY), such as comprises at least amino acids 2-9 (PAIQPPLYL), and having one or more amino acid substitutions, such as one amino acid substitution, such as two amino acid substitutions, such as three amino acid substitutions, such as four amino acid substitutions, such as five amino acid substitutions within said fragment.

16. The compound according to any one of the preceding claims, wherein P comprises or consists of an amino acid sequence of a MuV SH protein 2-57 selected from the group consisting of SEQ ID NO: 63 to SEQ ID NO: 73, or a variant thereof, or a fragment thereof, or a variant of a fragment thereof.

17. The compound according to any one of the preceding claims, wherein the amino acid sequence of P further comprises a methionine at the N-terminal end.

18. The compound according to any one of the preceding claims, wherein P comprises or consists of an amino acid sequence of a MuV SH protein 1-57 selected from the group consisting of SEQ ID NO: 74 to SEQ ID NO: 84, or a variant thereof, or a fragment thereof, or a variant of a fragment thereof.

19. The compound according to any one of the preceding claims, wherein P comprises or consists of an amino acid sequence selected from the group consisting of SEQ ID NO: 4, SEQ ID NO: 5, SEQ ID NO: 6, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, and a variant, a fragment, or a variant of a fragment of any one of SEQ ID NO: 4, SEQ ID NO: 5, SEQ ID NO: 6, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, or SEQ ID NO: 85.

20. The compound according to any one of the preceding claims, wherein P comprises or consists of a fragment of the MuV SH protein, such as comprises or consists of an N-terminal fragment of the MuV SH protein.

21. The compound according to any one of the preceding claims, wherein P comprises or consists of an amino acid sequence selected from the group consisting of SEQ ID NO: 7 to SEQ ID NO:62 and SEQ ID NO:85 to SEQ ID NO: 98, or a variant thereof.

22. The compound according to claim 21, wherein P is selected from the group consisting of SEQ ID NO: 7 to SEQ ID NO:62 and SEQ ID NO:85 to SEQ ID NO: 98, or a variant thereof having one or more amino acid substitutions, such as one amino acid substitution, such as two amino acid substitutions, such as three amino acid substitutions, such as four amino acid substitutions, such as five amino acid substitutions.

23. The compound according to any one of the preceding claims, wherein P is an N-terminal fragment of SEQ ID NO:1 selected from the group consisting of MPAIX₁PPX₂X₃X₄ TFLLL (SEQ ID NO: 7), MPAIX₁PPX₂X₃X₄ TELL (SEQ ID NO: 10), MPAIX₁PPX₂X₃X₄ TFL (SEQ ID NO: 13), MPAIX₁PPX₂X₃X₄ TF (SEQ ID NO: 16), MPAIX₁PPX₂X₃X₄ T (SEQ ID NO: 19), MPAIX₁PPX₂X₃X₄ (SEQ ID NO: 22), MPAIX₁PPX₂X₃ (SEQ ID NO: 25), MPAIX₁PPX₂ (SEQ ID NO: 27), MPAIX₁PP (SEQ ID NO: 29), MPAIX₁P (SEQ ID NO: 31), MPAIX₁ (SEQ ID NO: 33), PAIX₁PPX₂X₃X₄ TFLLL (SEQ ID NO: 35), PAIX₁PPX₂X₃X₄ TELL (SEQ ID NO: 38), PAIX₁PPX₂X₃X₄ TFL (SEQ ID NO: 41), PAIX₁PPX₂X₃X₄ TF (SEQ ID NO: 44), PAIX₁PPX₂X₃X₄ T (SEQ ID NO: 47), PAIX₁PPX₂X₃X₄ (SEQ ID NO: 50), PAIX₁PPX₂X₃ (SEQ ID NO: 53), PAIX₁PPX₂ (SEQ ID NO: 55), PAIX₁PP (SEQ ID NO: 57), PAIX₁P (SEQ ID NO: 59), and PAIX₁ (SEQ ID NO: 61),
wherein:
X₁ is Q or R
X₂ is L or S
X₃ is Y or H, and
X₄ is L or P.

24. The compound according to any one of the preceding claims, wherein P comprises or consists of a fragment of MuV SH protein having a length of less than 50 consecutive amino acid residues, for example 45 consecutive amino acid residues, such as 40 consecutive amino acid residues, for example less than 35 consecutive amino acid residues, such as less than 30 consecutive amino acid residues, for example less than 25 consecutive amino acid residues, such as less than 24 consecutive amino acid residues, for example less than 23 consecutive amino acid residues, such as less than 22 consecutive amino acid residues, for example less than 21 consecutive amino acid residues, such as less than 20 consecutive amino acid residues, for example less than 15 consecutive amino acid residues, such as less than 14 consecutive amino acid residues, for example less than 13 consecutive amino acid residues, such as less than 12 consecutive amino acid residues, for example less than 11 consecutive amino acid residues, such as less than consecutive 10 amino acid residues, for example less than 9 consecutive amino acid residues, such as less than 8 consecutive amino acid residues, for example less than 7 consecutive amino acid residues, such as less than 6 consecutive amino acid residues.

25. The compound according to any one of the preceding claims, wherein P comprises or consists of the 50 N-terminal consecutive amino acid residues of the MuV SH protein, for example the 45 N-terminal consecutive amino acid residues, such as the 40 N-terminal consecutive amino acid residues, for example comprises or consists of the 35 N-terminal consecutive amino acid residues, such as comprises or consists of the 30 N-terminal consecutive amino acid residues, for example comprises or consists of the 25 N-terminal consecutive amino acid residues, such as comprises or consists of the 24 N-terminal consecutive amino acid residues, for example comprises or consists of the 23 N-terminal consecutive amino acid residues, such as comprises or consists of the 22 N-terminal consecutive amino acid residues, for example comprises or consists of the 21 N-terminal consecutive amino acid residues, such as comprises or consists of the 20 N-terminal consecutive amino acid residues, for example comprises or consists of the 15 N-terminal consecutive amino acid residues, such as comprises or consists of the 14 N-terminal consecutive amino acid residues, for example comprises or consists of the 13 N-terminal consecutive amino acid residues, such as comprises or consists of the 12 N-terminal consecutive amino acid residues, for example comprises or consists of the 11 N-terminal consecutive amino acid residues, such as comprises or consists of the 10 N-terminal consecutive amino acid residues, for example comprises or consists of the 9 N-terminal consecutive amino acid residues, such as comprises or consists of the 8 N-terminal consecutive amino acid residues, for example comprises or consists of the 7 N-terminal consecutive amino acid residues, such as comprises or consists of the 6 N-terminal consecutive amino acid residues, for example comprises or consists of the 5 N-terminal consecutive amino acid residues of the MuV SH protein, such as of a MuV SH protein selected from the group consisting of SEQ ID NO: 1 to SEQ ID NO:6 and SEQ ID NO: 63 to SEQ ID NO: 84.

26. The compound according to any one of the preceding claims, wherein P comprises or consists of 15 or less than the 15 N-terminal consecutive amino acid residues of the MuV SH protein, such as of a MuV SH protein selected from the group consisting of SEQ ID NO: 1 to SEQ ID NO: 6 and SEQ ID NO: 63 to SEQ ID NO: 84.

27. The compound according to any one of the preceding claims, wherein P comprises or consists of 5 to 15 N-terminal consecutive amino acid residues of the MuV SH protein, such in 5, 6, 7, 8, 9, 10, 11, 12, 13, 14 or 15 N-terminal consecutive amino acid residues of the MuV SH protein, such as of a MuV SH protein selected from the group consisting of SEQ ID NO: 1 to SEQ ID NO:6 and SEQ ID NO: 63 to SEQ ID NO: 84.

28. The compound according to any one of the preceding claims, wherein P comprises or consists of the 9 N-terminal consecutive amino acid residues of the MuV SH protein, such as of a MuV SH protein selected from the group consisting of SEQ ID NO: 4 to 6 and SEQ ID NO: 74 to 84 (MuV SH protein 1-9).

29. The compound according to any one of the preceding claims, wherein P comprises or consists of the 8 N-terminal consecutive amino acid residues of the MuV SH protein, such as the 8 N-terminal consecutive amino acid residues of a MuV SH protein selected from the group consisting of SEQ ID NO: 1 to 3 and SEQ ID NO: 63 to 73 (MuV SH protein 2-9).

30. The compound according to any one of the preceding claims, wherein P comprises or consists of an amino acid sequence selected from the group consisting of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, and SEQ ID NO: 62, or a variant thereof.

31. The compound according to any one of the preceding claims, wherein P comprises or consists of an amino acid sequence selected from the group consisting of MPAIQPPLYL TFLLL (SEQ ID NO: 85), MPAIQPPLYL TELL (SEQ ID NO: 86), MPAIQPPLYL TFL (SEQ ID NO: 87), MPAIQPPLYL TF (SEQ ID NO: 88), MPAIQPPLYL T (SEQ ID NO: 89), MPAIQPPLYL (SEQ ID NO: 90), MPAIQPPLY (SEQ ID NO: 91), MPAIQPPL (SEQ ID NO: 28), MPAIQPP (SEQ ID NO: 30), MPAIQP (SEQ ID NO: 32), MPAIQ (SEQ ID NO: 34), PAIQPPLYL TFLLL (SEQ ID NO: 92), PAIQPPLYL TELL (SEQ ID NO: 93), PAIQPPLYL TFL (SEQ ID NO: 94), PAIQPPLYL TF (SEQ ID NO: 95), PAIQPPLYL T (SEQ ID NO: 96), PAIQPPLYL (SEQ ID NO: 97), PAIQPPLY (SEQ ID NO: 98), PAIQPPL (SEQ ID NO: 56), PAIQPP (SEQ ID NO: 58), PAIQP (SEQ ID NO: 60), and PAIQ (SEQ ID NO: 62), or a variant thereof, such as wherein said variant comprises one amino acid substitution, such as two amino acid substitutions, such as three amino acid substitutions.

32. The compound according to any one of the preceding claims, wherein P is selected from the group consisting of SEQ ID NO: 7 to SEQ ID NO: 62 and SEQ ID NO: 85 to SEQ ID NO: 98, or a variant thereof, wherein said variant comprises one or more amino acid substitutions.

33. The compound according to any one of the preceding claims, wherein P is selected from the group consisting of SEQ ID NO: 7 to SEQ ID NO: 62 and SEQ ID NO: 85 to SEQ ID NO: 98, or a variant thereof, wherein said variant comprises one amino acid substitution, such as two amino acid substitutions, such as three amino acid substitutions, such as four amino acid substitutions.

34. The compound according to any one of the preceding claims, wherein P is a N-terminal fragment derived from a MuV SH protein, such as derived from the MuV SH protein as set forth in SEQ ID NO: 74-84 or 115.

35. The compound according to any one of the preceding claims, wherein P comprises or consist of the sequence PAIQPPLY (SEQ ID NO: 98), or a variant thereof, such as a variant comprising one amino acid substitution, such as comprising two amino acid substitutions.

36. The compound according to any one of the preceding claims, wherein P comprises at least the sequence PAIQPPLY (SEQ ID NO: 98) and comprises or consists of in the range of the 5 to 20 consecutive amino acid residues of a MuV SH protein, such as in the range of the 5-6, 6-7, 7-8, 8-9, 9-10, 10-11, 11-12, 12-13, 13-14, 14-15, 15-16, 16-17, 17-18, 18-19 or 19-20 consecutive amino acid residues of the MuV SH protein.

37. The compound according to any one of the preceding claims, wherein P comprises at least the sequence PAIQPPLY (SEQ ID NO: 98), wherein P is selected from the group consisting of:
(SEQ ID NO: 98)
PAIQPPLY,
(SEQ ID NO: 97)
PAIQPPLYL,
(SEQ ID NO: 96)
PAIQPPLYLT,
(SEQ ID NO: 95)
PAIQPPLYLTF,
(SEQ ID NO: 94)
PAIQPPLYLTFL,
(SEQ ID NO: 93)
PAIQPPLYLTFLL,
(SEQ ID NO: 92)
PAIQPPLYLTFLLL,
(SEQ ID NO: 116)
PAIQPPLYLTFLLLI,
(SEQ ID NO: 117)
PAIQPPLYLTFLLLIL,
(SEQ ID NO: 118)
PAIQPPLYLTFLLLILL,
(SEQ ID NO: 119)
PAIQPPLYLTFLLLILLY,
(SEQ ID NO: 120)
PAIQPPLYLTFLLLILLYL,
and
(SEQ ID NO: 121)
PAIQPPLYLTFLLLILLYLI,
or a variant thereof.

38. The compound according to any one of the preceding claims, wherein P comprises at least the sequence PAIQPPLY (SEQ ID NO: 98), wherein P is selected from the group consisting of:
(SEQ ID NO: 91)
MPAIQPPLY,
(SEQ ID NO: 90)
MPAIQPPLYL,
(SEQ ID NO: 89)
MPAIQPPLYLT,
(SEQ ID NO: 88)
MPAIQPPLYLTF,
(SEQ ID NO: 87)
MPAIQPPLYLTFL,
(SEQ ID NO: 86)
MPAIQPPLYLTFLL,
(SEQ ID NO: 85)
MPAIQPPLYLTFLLL,
(SEQ ID NO: 122)
MPAIQPPLYLTFLLLI,
(SEQ ID NO: 123)
MPAIQPPLYLTFLLLIL,
(SEQ ID NO: 124)
MPAIQPPLYLTFLLLILL,
(SEQ ID NO: 125)
MPAIQPPLYLTFLLLILLY,
and
(SEQ ID NO: 126)
MPAIQPPLYLTFLLLILLYL,
or a variant thereof.

39. The compound according to any one of the preceding claims, wherein the variant of P is a functional variant of P.

40. The compound according to any one of the preceding claims, wherein the variant of P or the functional variant of P is a peptide selected from the group consisting of any one of SEQ ID NO: 85-98 or 116-126 having one or more amino acid substitutions.

41. The compound according to any one of the preceding claims, wherein the variant of P or the functional variant of P is a peptide selected from the group consisting of any one of SEQ ID NO: 85-98 or 116-126 having one amino acid substitution or having two amino acid substitutions or having three amino acid substitutions or having four amino acid substitutions or having five amino acid substitutions.

42. The compound according to any one of the preceding claims, wherein the one or more amino acid substitutions are conservative amino acid substitutions.

43. The compound according to any one of the preceding claims, wherein D is a GLP-1 receptor agonist, such as a peptide-based GLP-1 receptor agonist.

44. The compound according to any one of the preceding claims, wherein D is a GLP-1 derived peptide, such as a GLP-1 analogue.

45. The compound according to any one of the preceding claims, wherein D is an Exendin-4 derived peptide, such as a Exendin-4 analogue.

46. The compound according to any one of the preceding claims, wherein D is a GLP-1 receptor agonist selected from the group consisting of exenatide, liraglutide, lixisenatide, albiglutide, dulaglutide, and semaglutide.

47. The compound according to any one of the preceding claims, wherein D is GLP-1, such as native GLP-1, such as GLP-1(1-37) or a variant, a fragment, or a variant of a fragment thereof

48. The compound according to any one of the preceding claims, wherein D is GLP-1(7-37) (SEQ ID NO: 132), or a variant, a fragment, or a variant of a fragment thereof

49. The compound according to any one of the preceding claims, wherein D is GLP-1(7-36) (SEQ ID NO:127), or a variant, a fragment, or a variant of a fragment thereof.

50. The compound according to any one of the preceding claims, wherein said variant of GLP-1, GLP-1(7-36) or GLP-1 (7-37) comprises one or more amino acid substitutions, amino acid deletions, and/or an amino acid insertions.

51. The compound according to any one of the preceding claims, wherein said variant of GLP-1, GLP-1 (7-37) or GLP-1 (7-36) comprises one or more amino acid substitutions to the native sequence of GLP-1, to SEQ ID NO: 127 (GLP-1 7-36) or to SEQ ID NO: 132 (GLP-1 7-37).

52. The compound according to any one of the preceding claims, wherein said variant of GLP-1, GLP-1 (7-37) or GLP-1 (7-36) comprises one amino acid substitution, such as two amino acid substitutions, such as three amino acid substitutions, such as 4 amino acid substitutions, such as 5 amino acid substitutions, such as 6 amino acid substitutions, such as 7 amino acid substitutions, such as 8 amino acid substitutions to the native sequence of GLP-1, such as to SEQ ID NO: 127 (GLP-1 7-36) or to SEQ ID NO: 132 (GLP-1 7-37).

53. The compound according to any one of the preceding claims, wherein the one or more amino acid substitution(s) is a conservative amino acid substitution.

54. The compound according to any one of the preceding claims, wherein said variant of GLP-1, GLP-1 (7-37) or GLP-1 (7-36) comprises one or more fatty acid moieties.

55. The compound according to any one of the preceding claims, wherein said GLP-1, GLP-1 (7-37) or GLP-1 (7-36), or variants thereof, is C-terminally amidated.

56. The compound according to any one of the preceding claims, wherein said variant of GLP-1 comprises an amino acid substitution wherein a Lys is introduced at any position in the GLP-1 sequence.

57. The compound according to any one of the preceding claims, wherein said variant of GLP-1 contains a Lys at any position selected from position 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and 37 of GLP-1.

58. The compound according to any one of the preceding claims, wherein said GLP-1 or said variant of a GLP-1 is conjugated to P via a linker (L).

59. The compound according to any one of the preceding claims, wherein said GLP-1 or said variant of a GLP-1 is conjugated to the MuV SH protein or a variant, a fragment, or a variant of a fragment thereof via a linker (L).

60. The compound according to any one of the preceding claims, wherein said GLP-1 or said variant of GLP-1 comprises a linker or a spacer on Lys6, Lys7, Lys8, Lys9, Lys10, Lys11, Lys12, Lys13, Lys14, Lys15, Lys16, Lys17, Lys18, Lys19, Lys20, Lys21, Lys22, Lys23, Lys24, Lys25, Lys26, Lys27, Lys28, Lys29, Lys30, Lys31, Lys32, Lys33, Lys34, Lys35, Lys36 or Lys37 of said GLP-1.

61. The compound according to any one of the preceding claims, wherein D is GLP-1 or a variant, a fragment, or a variant of a fragment thereof containing a naturally occurring Lys, such as Lys26 or Lys34 of GLP-1.

62. The compound according to any one of the preceding claims, wherein D is GLP-1 or a variant, a fragment, or a variant of a fragment thereof containing an artificially introduced Lys, such as Lys26 or Lys34 of GLP-1.

63. The compound according to any one of the preceding claims, wherein D is GLP-1 or a variant, a fragment, or a variant of a fragment thereof and is conjugated to the MuV SH protein or a variant, a fragment, or a variant of a fragment thereof (P) via a linker (L), such as via a linker (L) on a lysine residue of said GLP-1 protein.

64. The compound according to any one of the preceding claims, wherein D is GLP-1 or a variant, a fragment, or a variant of a fragment thereof and is conjugated to the MuV SH protein or a variant, a fragment, or a variant of a fragment thereof via a linker (L), such as via a linker (L) on Lys26 or Lys34 of GLP-1.

65. The compound according to any one of the preceding claims, wherein D is GLP-1(7-36) (SEQ ID NO: 127), or a variant thereof, optionally comprising a linker or a spacer on a lysine residue such as on Lys26.

66. The compound according to any one of the preceding claims, wherein D is GLP-1(7-37) (SEQ ID NO:132) optionally comprising a linker or a spacer on a lysine residue such as on Lys26.

67. The compound according to any one of the preceding claims, wherein D is GLP-1(7-36) (SEQ ID NO: 127) or GLP-1(7-37) (SEQ ID NO:132), and comprises a linker or a spacer on a lysine residue such as on Lys26, wherein said linker is a PEG or PEG2 linker.

68. The compound according to any one of the preceding claims, wherein the compound comprises or consist of SEQ ID NO: 128, or a variant thereof.

69. The compound according to any one of the preceding claims, wherein D is Exendin-4 or Exendin-4(1-39) (SEQ ID NO:130) or a variant, a fragment, or a variant of a fragment thereof.

70. The compound according to any one of the preceding claims, wherein said Exendin-4 is C-terminally amidated.

71. The compound according to any one of the preceding claims, wherein the variant of Exendin-4 comprises one or more amino acid substitutions, one or more amino acid deletions, and/or one or more amino acid insertions.

72. The compound according to any one of the preceding claims, wherein the variant of Exendin-4 comprises one or more amino acid substitutions, such as one amino acid substitution, such as two amino acid substitutions, such as three amino acid substitutions, such as 4 amino acid substitutions, such as 5 amino acid substitutions, such as 6 amino acid substitutions, such as 7 amino acid substitutions, such as 8 amino acid substitutions.

73. The compound according to any one of the preceding claims, wherein the one or more amino acid substitution(s) is a conservative amino acid substitution.

74. The compound according to any one of the preceding claims, wherein the variant of Exendin-4 comprises an amino acid substitution wherein a Lys is introduced at any position in the Exendin-4 sequence.

75. The compound according to any one of the preceding claims, wherein said Exendin-4 or variant thereof contains a Lys at any position selected from position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 and 39 of SEQ ID NO:130.

76. The compound according to any one of the preceding claims, wherein D is Exendin-4 or a variant, a fragment, or a variant of a fragment thereof and is conjugated to the MuV SH protein or a variant, a fragment, or a variant of a fragment thereof (P) via a linker (L), such as via a linker (L) on a lysine residue of said Exendin-4 protein.

77. The compound according to any one of the preceding claims, wherein D is Exendin-4 or a variant, a fragment, or a variant of a fragment thereof comprising a linker or a spacer on Lys1, Lys2, Lys3, Lys4, Lys5, Lys6, Lys7, Lys8, Lys9, Lys10, Lys11, Lys12, Lys13, Lys14, Lys15, Lys16, Lys17, Lys18, Lys19, Lys20, Lys21, Lys22, Lys23, Lys24, Lys25, Lys26, Lys27, Lys28, Lys29, Lys30, Lys31, Lys32, Lys33, Lys34, Lys35, Lys36, Lys37, Lys38 or Lys39.

78. The compound according to any one of the preceding claims, wherein D is Exendin-4 or a variant, a fragment, or a variant of a fragment thereof containing a naturally occurring Lys, such as Lys12 or Lys27.

79. The compound according to any one of the preceding claims, wherein D is Exendin-4 or a variant, a fragment, or a variant of a fragment thereof containing an artificially introduced Lys, such as Lys12 or Lys27.

80. The compound according to any one of the preceding claims, wherein D is Exendin-4 or a variant, a fragment, or a variant of a fragment thereof and is conjugated to the MuV SH protein or a variant, a fragment, or a variant of a fragment thereof via a linker (L), such as via a linker (L) on Lys12 or Lys27 of said Exendin-4.

81. The compound according to any one of the preceding claims, wherein D is Exendin-4(1-39) (SEQ ID NO: 130), optionally comprising a linker or a spacer on Lys27.

82. The compound according to any one of the preceding claims, wherein D is Exendin-4(1-39) comprising a linker or a spacer on Lys27, such as a PEG or PEG2 linker.

83. The compound according to any one of the preceding claims, wherein the compound comprises or consist of SEQ ID NO: 131, or a variant thereof.

84. The compound according to any one of the preceding claims, wherein P is capable of interacting with G-protein coupled receptor 125 (ADGRA3/GPR125), such as capable of binding to GPR-125.

85. The compound according to any one of the preceding claims, wherein D is a peptide based or protein based drug D, and P is covalently attached to D at the N-terminus of D, at the C-terminus of D, or at an amino acid side chain of D, optionally via a linker L.

86. The compound according to any one of the preceding claims, wherein D is covalently attached to P at the N-terminus of P, at the C-terminus of P, or at an amino acid side chain of P, optionally via a linker L.

87. The compound according to any one of the preceding claims, wherein D is covalently attached to P at the C-terminus of P, optionally via a linker L.

88. The compound according to any one of the preceding claims, wherein D is covalently attached to P via a linker L.

89. The compound according to any one of the preceding claims, wherein the compound comprises a structure according to formula (II) 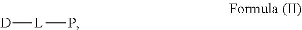
wherein
D comprises or consists of a drug;
P comprises or consists of a mumps virus small hydrophobic protein (MuV SH protein) or a variant, a fragment, or a variant of a fragment thereof; and
L is a linker
wherein D is conjugated to P via the linker L.

90. The compound according to any one of the preceding claims, wherein L is a non-cleavable linker.

91. The compound according to any one of the preceding claims, wherein L is a cleavable linker.

92. The linker according to claim 91, wherein the cleavable linker is selected from the group consisting of acid cleavable linkers, pH cleavable linker, and enzyme cleavable linker.

93. The compound according to any one of the preceding claims, wherein L is selected from the group consisting of polyethylene glycol linker (PEG linker), Glycine serine linker (GS linker).

94. The compound according to any one of the preceding claims, wherein L is selected from the group consisting of PEG2-20, aliphatic spacers with a length from 4-60 atoms, linear and branched amine spacers such as HMDA, linear and branched amide spacers and peptide spacers such as Glutathione (GSH).

95. The compound according to any one of the preceding claims, wherein L is attached to a naturally occurring Lys in the drug D or wherein L is attached to a Lys which has been artificially introduced in the drug D.

96. The compound according to any one of the preceding claims, wherein D is a pharmaceutical drug.

97. The compound according to any one of the preceding claims wherein D comprises or consists of a drug capable of targeting a component in the central nervous system (CNS).

98. The compound according to any one of the preceding claims, wherein D is selected from the group consisting of antidepressants, anticancer agents, painkiller, antidiabetic drugs, neurodegenerative drugs, anti-hypertensive drugs, diuretic drugs, and anti-inflammatory drugs.

99. The compound according to any one of the preceding claims, wherein the compound is capable of interacting with G-protein coupled receptor 125 (GPR125), such as capable of interacting with GPR125 via binding of P to GPR125.

100. The compound according to any one of the preceding claims, wherein interaction of the compound with GPR125 facilitates crossing of the compound across a membrane or barrier or barrier harbouring the GPR125.

101. The compound according to any one of the preceding claims, wherein interaction of the compound with GPR125 facilitates internalization of the compound across a membrane or barrier harbouring the GPR125.

102. The compound according to any one of the preceding claims, wherein interaction of the compound with GPR125 results in reduced membrane integrity of the membrane harbouring the GPR125.

103. The compound according to any one of the preceding claims, wherein the membrane or barrier harbouring GPR125 is selected from the group consisting of choroid plexus, kidney epithelium, urogenital epithelium, blood-ocular barriers, and lung epithelium.

104. The compound according to any one of the preceding claims, wherein the membrane or barrier harbouring GPR125 is the choroid plexus.

105. The compound according to any one of the preceding claims, wherein the compound is capable of crossing of the blood brain barrier, such as the blood-cerebrospinal fluid barrier, for example the choroid plexus.

106. The compound according to any one of the preceding claims for use in targeted delivery of D across a membrane or barrier harbouring GPR125 and/or to a site expressing GPR125.

107. The compound according to any one of the preceding claims for use in targeted delivery of D to the cerebrospinal fluid.

108. The compound according to any one of the preceding claims, wherein interaction of the compound with GPR125 facilitates targeted delivery of D to a tumour, such as to a tumour present in the CNS, for example to a brain tumour, such as to a brain tumour selected from the group consisting of astrocytic tumour, oligodendroglial tumour, glioblastoma, pituitary tumour, pineal gland tumour, ependymomas, craniopharygiomas, primary central nervous system lymphomas, meningiomas, and schwannomas.

109. The compound according to any one of the preceding claims, wherein the compound does not comprise a linker.

110. The compound according to any one of the preceding claims for use in targeted delivery of D to the CNS, such as to the brain.

111. The compound according to any one of the preceding claims for use in targeted delivery of D to the eyeball.

112. The compound according to any one of the preceding claims for use as a medicament.

113. The compound according to any one of the preceding claims for use in the treatment of a CNS disease or disorder, a kidney disease or disorder, a urogenital disease or disorder, a metabolic disorder, an ocular disease or disorder, brain/CNS metastasis, or a lung disease or disorder.

114. The compound for use according to any one of the preceding claims, wherein the CNS disease or disorder is selected from the group consisting of neurodegenerative diseases, mental disorders, infectious disorder, headache or migraine, brain tumours, and brain/CNS metastasis.

115. The compound for use according to any one of the preceding claims, wherein the CNS disease is a neurodegenerative disorder selected from the group consisting of Alzheimer's disease, Parkinson's disease, amyotrophic lateral sclerosis (ALS), Huntington's disease, and multiple sclerosis; a mental disorder selected from the group consisting of dementia, depression, schizophrenia, bipolar disorder, anxiety disorder, eating disorder, and addiction; an infectious disorder selected from the group consisting of meningitis and encephalitis; headache or migraine; a brain tumour selected from the group consisting of astrocytic tumour, oligodendroglial tumour, glioblastoma, pituitary tumour, pineal gland tumour, ependymomas, craniopharygiomas, primary central nervous system lymphomas, meningiomas, and schwannomas; or brain/CNS metastasis.

116. The compound for use according to any one of the preceding claims, wherein the disease is a metabolic disorder selected from the group consisting of metabolic disease, type 2 diabetes, and obesity.

117. The compound according to any one of the preceding claims for use in the treatment of obesity.

118. The compound according to any one of the preceding claims for use in a method of appetite regulation such as appetite reduction; such as for use in a method of reducing food intake.

119. A method of preparing a drug which is permeable to the blood brain barrier, such as permeable to the blood-cerebrospinal fluid barrier, such as the choroid plexus, the method comprising covalently conjugating the drug to a mumps virus short hydrophobic protein (MuV SH protein) or a variant of fragment thereof, optionally via a linker, as defined in any one of the preceding claims.

120. A method for delivering a drug across a membrane or barrier harbouring GPR125, such as across the choroid plexus, the method comprising providing a compound comprising said drug as defined in any one of the preceding claims.

121. The method according to claim 120, wherein the method is prepared in vitro or ex vivo.

122. The method according to claim 120-121, wherein said method is for targeted delivery of said compound to the brain and/or other central nervous system tissue.
